(19) United States
(12) Patent Application Publication
(10) Pub. No.: US 2023/0390287 A1
(43) Pub. Date: Dec. 7, 2023

(54) MODULATORS OF PD-L1/PD-1 INTERACTION AND USES THEREOF

(71) Applicant: Ramot at Tel-Aviv University Ltd., Tel-Aviv (IL)

(72) Inventors: Ronit SATCHI-FAINARO, Tel-Aviv (IL); Helena FLORINDO, Tel-Aviv (IL); Rita GUEDES, Tel-Aviv (IL); Rita ACÚRCIO, Tel-Aviv (IL)

(73) Assignee: Ramot at Tel-Aviv University Ltd., Tel-Aviv (IL)

(21) Appl. No.: 18/235,392

(22) Filed: Aug. 18, 2023

Related U.S. Application Data
(63) Continuation of application No. PCT/IL2022/050195, filed on Feb. 18, 2022.
(60) Provisional application No. 63/150,643, filed on Feb. 18, 2021.

(51) Int. Cl. A61K 31/506; A61K 31/428; A61K 31/351; A61K 31/135; A61K 31/44

(52) U.S. Cl. CPC A61K 31/506; A61K 31/428; A61K 31/44; A61K 31/135; A61K 31/351

(57) ABSTRACT

Compounds (small molecules) capable of interfering with an interaction between PD-1 and PD-L1, and thereby are usable in treating cancer and/or in increasing T-cell function and/or in treating a medical condition associate with PD1, PD-L1 and/or T-cell function on cells, pharmaceutical compositions and kits comprising same and uses thereof, are provided.

### RELATED APPLICATIONS

This application is a Continuation of PCT Patent Application No. PCT/IL2022/050195 having International filing date of Feb. 18, 2022 which claims the benefit of priority under 35 USC § 119(e) of U.S. Provisional Patent Application No. 63/150,643 filed on Feb. 18, 2021. The contents of the above applications are all incorporated by reference as if fully set forth herein in their entirety.

### FIELD AND BACKGROUND OF THE INVENTION

The present invention, in some embodiments thereof, relates to therapy and, more particularly, but not exclusively, to small molecule compounds which are capable of interfering with PD-1/PD-L1 interaction, and which are usable in treating conditions associated with a PD-1 and/or PD-L1 function, and/or which are treatable by enhancing T-cell function, such as cancer, neurodegenerative diseases and infectious diseases.

Cancer immunotherapy, defined as the targeting or use of immune system components to kill tumor cells, has revolutionized the treatment of advanced-stage malignancies. Among several immunotherapeutic approaches that have been tested in the last decade, immune checkpoint blockade is now a clinical reality with remarkable results.

Immune checkpoint receptors are primary regulators of T-cell function that define the balance between tolerance and autoimmunity. These receptors act as homeostatic controllers of the immune system response. However, within the tumor microenvironment (TME), tumors hijack immune checkpoint receptors to suppress T-cell function. Based on this observation, the inhibition of these receptors has been highly explored to restore exhausted T-cell function and reactivate the immune system to recognize and kill tumor cells.

Studies have shown that the inhibition of programmed cell death protein 1 (PD-1) or PD-ligand (PD-L1) has prevented or reversed exhausted T cells, thus enhancing antitumor T-cell responses [Kamphorst, A. O. & Ahmed, R. Curr. Opin. Immunol. 25, 381-388 (2013); Pauken, K. E. & Wherry, E. J. Trends Immunol. 36, 265-276 (2015); Wei, S. C. et al. Cell 170, 1120-1133.e17 (2017)]. In fact, immune checkpoint blockade targeting the PD-1 axis has become one of the most effective therapies for diverse cancers [Brahmer, J. R. et al. N. Engl. J. Med. 366, 2455-2465 (2012); Sonpavde, G. N. Engl. J. Med. 376, 1073-1074 (2017)].

So far, only monoclonal antibodies are approved as antibody-based PD-1/PD-L1 inhibitors. However, their overall efficiency, the lack of understanding of the mechanistic basis regulating this immune checkpoint pathway results in low response rates (about 25%), absence of long-term remission, and severe immune-related adverse events (IRAE). In addition, monoclonal antibodies (mAb) are very expensive to produce, which in addition to their required intravenous administration, results in high-cost treatment regimens which are financially inaccessible to many. These limitations preclude that the full potential of the immune checkpoint blockade has yet to be fulfilled.

Recently, efforts have focused on the development of small-molecule inhibitors as an alternative approach to therapeutically target PD-L1 or PD-1 [Adams et al. Nat. Rev. Drug Discov. 14, 603-22 (2015); Weinmann, H. ChemMedChem 11, 450-466 (2016)]. The use of small molecules offers several unique advantages over mAb drugs. Small molecules can provide increased oral bioavailability, bio-efficiency, and short half-life activity, particularly relevant for IRAE [Zhan, M. M. et al. Drug Discov. Today 21, 1027-1036 (2016); Barakat, K. J. Pharm. Care Heal. Syst. 01, 4-5 (2014)]. In addition, small molecules can offer a greater diffusion rate within the TME, target PD-L1 within other cellular sources, including at the intracellular level, and the possibility of avoiding the macrophage-mediated resistance observed in anti-PD-1 therapy [Arlauckas, S. P. et al. Sci. Transl. Med. 9, eaa13604 (2017)].

Studies related to the development of small molecule-inhibitors targeting PD-1/PD-L1 have been described, for example, in Abdel-Magid, A. F. ACS Med. Chem. Lett. 6, 489-490 (2015); Guzik, K. et al. J. Med. Chem. 1, acs.jmedchem.7b00293 (2017); Skalniak, L. et al. Oncotarget 8, 72167-72181 (2017); Acnrcio, R. C. et al. Medchemcomm 10, 1810-1818 (2019); Zak, K. M. et al. Structure 23, 2341-2348 (2015); Zak, K. M. et al. Oncotarget 7, 30323-30335 (2016); Qin, M. et al. J. Med. Chem. 62, 4703-4715 (2019); Blevins, D. J. et al. ACS Med. Chem. Lett. 10, 1187-1192 (2019); Acnrcio, R. C. et al, J. Med. Chem. 61, 10957-10975 (2018); and Acnrcio, R. C. et al., WIREs Comput Mol Sci. 9, e1397 (2018).

However, to date, effective small-molecule inhibitors targeting PD-1/PD-L1, or any other immune checkpoint receptor or related immunosuppressor targets (e.g., TGF-β), are limited and have not reached the clinic yet.

### SUMMARY OF THE INVENTION

Immune checkpoint blockade is one of the most effective approaches to cancer immunotherapy. Inhibiting programmed cell death protein 1 (PD-1) or PD-ligand 1 (PD-L1) has shown exciting clinical outcomes in diverse human cancers. Despite the exciting outcomes, most patients do not benefit from the currently available immune checkpoint inhibitors, and many develop severe immune-related adverse events. So far, only monoclonal antibodies are approved as immune checkpoint modulators. However, revolutionary strategies involving the design of small molecules are emerging to improve the outcomes of immune checkpoint blockade therapies.

The present inventors have followed a trans-disciplinary approach to discover novel small molecules that can modulate PD-1/PD-L1 interaction. The present inventors have employed in silico analyses combined with in vitro, ex vivo and in vivo experimental studies to assess the ability of newly uncovered compounds to modulate PD-1/PD-L1 interaction and enhance T-cell function. These studies have led to the identification of small molecules that are capable of promoting T-cell activation by targeting the PD-1/PD-L1 co-inhibitory interactions, which proved to be as effective as monoclonal antibodies. The newly-identified small molecules enabled an extensive infiltration of T lymphocytes into 3D solid tumor models, as well as cytotoxic T CD8+ lymphocytes within solid tumor mass in vivo, thus presenting high potential to revolutionize cancer immunotherapy.

Embodiments of the present invention relate to small molecules (compounds) that are usable as modulators (e.g., inhibitors) of PD-1/PD-L1 interaction and in enhancing T-cell function, and are therefore usable as immune checkpoint inhibitors and in treating medical conditions that are treatable by immune checkpoint blockade, e.g., by inhibition of PD-1/PD-L1 interaction, such as, for example, cancer.

According to an aspect of some embodiments of the present invention there is provided a compound represented by Formula I:

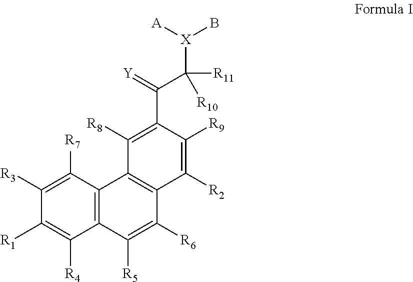

or a pharmaceutically acceptable salt thereof,
wherein:
R₁-R₁₁ are each independently selected from hydrogen, alkyl, cycloalkyl, aryl, heteroaryl, heteroalicyclic, alkoxy, aryloxy, hydroxy, thiol, thioalkoxy, thioaryloxy, amine, imine, halo, nitro, nitrile (cyano), amide, hydrazine, hydrazide, carboxylate, thiocarboxylate, carbamate, thiocarbamate, sulfonyl, sulfinyl, sulfonamide, carbonate, thiocarbonate, sulfoxide, thiosulfate, sulfate, sulfite, thiosulfite, phosphonate, urea, thiourea, guanyl and guanidyl;
Y is O or S;
X is O, S or N, wherein when X is O or S, B is absent; and
A and B (if present) are each independently selected from hydrogen, alkyl, cycloalkyl, aryl, heteroaryl, and heteroalicyclic, the alkyl, cycloalkyl, aryl, heteroaryl, and heteroalicyclic being independently substituted (e.g., as described herein) or unsubstituted,
for use in treating cancer and/or for use in interfering with an interaction between PD1 and PD-L1 and/or for use in increasing T-cell function (e.g. TGF-β), and/or for use in treating a medical condition associate with PD1, PD-L1 and/or T-cell function on cells, in a subject in need thereof.

According to an aspect of some embodiments of the present invention there is provided a compound represented by Formula II:

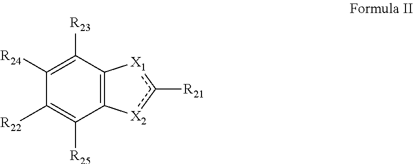

or a pharmaceutically acceptable salt thereof,
wherein:
X₁ and X₂ are each independently selected from N, NR₂₈, S, O, CR₂₆, and CR₂₆R₂₇, at least one of X₁ and X₂ being N, NR₂₈, S or O, and wherein each of the dashed lines represents an optional bond (forming a double bond) when the adjacent X₁ or X₂ is N or CR₂₆;
R₂₈ is hydrogen, alkyl, cycloalkyl or aryl; and
R₂₁-R₂₇ are each independently selected from hydrogen, alkyl, cycloalkyl, aryl, hydroxy, alkoxy, aryloxy, thiol, thioalkoxy, thioaryloxy, amine, imine, halo, nitrile (cyano), nitro, amide, hydrazine, hydrazide, carboxylate, thiocarboxylate, carbamate, thiocarbamate, sulfonyl, sulfinyl, sulfonamide, carbonate, thiocarbonate, sulfoxide, thiosulfate, sulfate, sulfite, thiosulfite, phosphonate, urea, thiourea, guanyl and guanidyl,
provided that at least one and preferably both of R₂₁ and R₂₂ is a heteroatom-containing moiety such as alkoxy, aryloxy, thiol, thioalkoxy, thioaryloxy, amine, imine, nitrile (cyano), amide, hydrazine, hydrazide, carboxylate, thiocarboxylate, carbamate, thiocarbamate, sulfonyl, sulfinyl, and/or sulfonamide,
for use in treating cancer and/or for use in interfering with an interaction between PD1 and PD-L1 and/or for use in increasing T-cell function (e.g. TGF-β), and/or for use in treating a medical condition associate with PD1, PD-L1 and/or T-cell function on cells, in a subject in need thereof.

According to an aspect of some embodiments of the present invention there is provided a compound represented by Formula III:

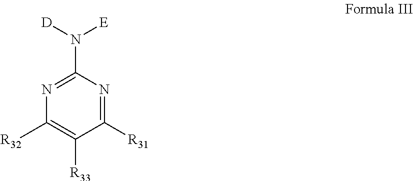

or a pharmaceutically acceptable salt thereof,
Wherein:
R₃₃ is hydrogen, alkyl, cycloalkyl, aryl, halo, amine, hydroxy, thiol, aryl, alkoxy, thioalkoxy, aryloxy, thioaryloxy, or, alternatively, forms a cyclic ring with R₃₁ or R₃₂;
R₃₁ and R₃₂ are each independently selected from hydrogen, halo, alkyl, aryl, and amine, or, alternatively, one of R₃₁ and R₃₂ forms a cyclic ring with R₃₃; and
D and E are each independently selected from hydrogen, alkyl, cycloalkyl, aryl, heteroaryl, carbonyl (carbonate), thiocarbonyl (thiocarbonate), carboxylate, thiocarboxylate, sulfonyl, sulfinyl, sulfoxide, thiosulfate, sulfate, sulfite, thiosulfite, urea, thiourea, guanyl and guanidyl,
wherein at least one or at least two of R₃₁-R₃₃, D and E is or comprises an aryl,
for use in treating cancer and/or for use in interfering with an interaction between PD1 and PD-L1 and/or for use in increasing T-cell function (e.g. TGF-β), and/or for use in treating a medical condition associate with PD1, PD-L1 and/or T-cell function on cells, in a subject in need thereof.

According to an aspect of some embodiments of the present invention there is provided a compound represented by Formula IV:

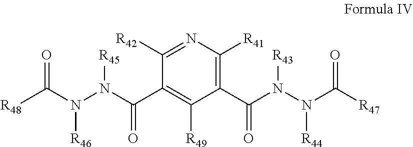

or a pharmaceutically acceptable salt thereof,
wherein:
R₄₃-R₄₆ are each independently hydrogen, alkyl, cycloalkyl and aryl;
R₄₁, R₄₂ and R₄₉ are each independently selected from hydrogen, alkyl, cycloalkyl, aryl, hydroxy, alkoxy, aryloxy, thiol, thioalkoxy, thioaryloxy, amine, imine, halo, nitrile (cyano), nitro, amide, hydrazine, hydrazide, carboxylate, thiocarboxylate, carbamate, thiocarbamate, sulfonyl, sulfinyl, sulfonamide, carbonate, thiocarbonate, sulfoxide, thiosulfate, sulfate, sulfite, thiosulfite, phosphonate, urea, thiourea, guanyl and guanidyl; and
R₄₇ and R₄₈ are each independently selected from alkyl, cycloalkyl, aryl, heteroaryl and heteroalicyclic,
for use in treating cancer and/or for use in interfering with an interaction between PD1 and PD-L1 and/or for use in increasing T-cell function (e.g. TGF-β), and/or for use in treating a medical condition associate with PD1, PD-L1 and/or T-cell function on cells, in a subject in need thereof.

According to an aspect of some embodiments of the present invention there is provided a compound selected from Compounds 1, 5, 18, 29, 42, 45, 47, 69, 71, 73, 75 and 84, as presented in Table B, for use in treating cancer and/or for use in interfering with an interaction between PD1 and PD-L1 and/or for use in increasing T-cell function (e.g. TGF-β), and/or for use in treating a medical condition associate with PD1, PD-L1 and/or T-cell function on cells, in a subject in need thereof.

According to an aspect of some embodiments of the present invention there is provided a compound selected from Compounds 5, 42, 47, 69, 75 and 84, as presented in Table B, for use in treating cancer and/or for use in interfering with an interaction between PD1 and PD-L1 and/or for use in increasing T-cell function (e.g. TGF-β), and/or for use in treating a medical condition associate with PD1, PD-L1 and/or T-cell function on cells, in a subject in need thereof.

According to some of any of the embodiments described herein, the compound is capable of interfering with an interaction between PD-1 and PD-L1.

According to some of any of the embodiments described herein, the cancer is characterized by overexpression of PD-1.

According to some of any of the embodiments described herein, the cancer is selected from lung cancer, melanoma, breast cancer, colorectal cancer and bladder cancer.

According to an aspect of some embodiments of the present invention there is provided a compound as described herein in any of the respective embodiments and any combination thereof is for use in treating a medical condition selected from a neurodegenerative disease or disorder and an infectious disease or disorder in a subject in need thereof.

According to an aspect of some embodiments of the present invention there is provided a method of identifying a lead candidate small molecule compound for treating cancer, the method comprising: Computationally (in silico) screening small molecules database interfering with an interaction between PD1 and PD-L1 to thereby identify small molecule compounds that are capable of interacting with a binding pocket of PD-L1; and Subjecting small molecule compounds identified as capable of interacting with a binding pocket of PD-L1 to an assay that determines inhibition of an interaction between FD-1 and PD-L1, to thereby identify compounds capable of inhibiting said interaction, wherein compounds identified as capable of inhibiting said interaction are determined as capable of treating cancer, thereby identifying a lead candidate small molecule compound for treating cancer.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Implementation of the method and/or system of embodiments of the invention can involve performing or completing selected tasks manually, automatically, or a combination thereof. Moreover, according to actual instrumentation and equipment of embodiments of the method and/or system of the invention, several selected tasks could be implemented by hardware, by software or by firmware or by a combination thereof using an operating system.

For example, hardware for performing selected tasks according to embodiments of the invention could be implemented as a chip or a circuit. As software, selected tasks according to embodiments of the invention could be implemented as a plurality of software instructions being executed by a computer using any suitable operating system. In an exemplary embodiment of the invention, one or more tasks according to exemplary embodiments of method and/or system as described herein are performed by a data processor, such as a computing platform for executing a plurality of instructions. Optionally, the data processor includes a volatile memory for storing instructions and/or data and/or a non-volatile storage, for example, a magnetic hard-disk and/or removable media, for storing instructions and/or data. Optionally, a network connection is provided as well. A display and/or a user input device such as a keyboard or mouse are optionally provided as well.

### BRIEF DESCRIPTION OF THE SEVERAL VIEWS OF THE DRAWING(S)

The patent or application file contains at least one drawing executed in color. Copies of this patent or patent application publication with color drawing(s) will be provided by the Office upon request and payment of the necessary fee.

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is a Ribbon representation created in Pymol software using the crystal structure of PD-L1 (PDB 5J89), as shown in Acnrcio, R. C. et al. Medchemcomm 10, 1810-1818 (2019), showing PD-L1 monomers (gray) bridged by small-molecule inhibitor BMS202 (yellow), and a close-up view of the binding pocket. Receptor-ligand interactions are displayed in dashes. Hydrophobic contacts (yellow), π-stacking (green) and H-bond and salt bridges (red).

FIGS. 2A-C describe the in silico virtual screening for putative PD-1/PD-L1 inhibitors, according to some of the present embodiments, described in Example 1. FIG. 2A (Background Art) presents the crystal structure of human PD-L1 (PDB 5J89) selected to be used in the in silico studies described in Example 1, as taken from Acnrcio, R. C. et al. Medchemcomm 10, 1810-1818 (2019). FIG. 2B is a schematic presentation of the various stages of the Structure-based virtual screening according to some of the present embodiments, employed for the identification of PD-1/PD-L1 small-molecule inhibitors. FIG. 2C is a schematic presentation of the identified compounds according to their chemical skeleton.

FIG. 3A is a bar graph showing the PD-1/PD-L1 inhibition by 16 validated hits (blue-1 and 73, black-5, 18, 29, 75, 84, green-42, 45 and 71, yellow-69 and 47, and white-30, 32, 35 and 38), obtained at 100 μM using homogeneous time-resolved fluorescence (HTRF). BMS202 (dark gray) was used as positive control for PD-1/PD-L1 inhibition. Results were normalized (0-100%) considering PD-1/PD-L1 interaction (light gray) the 100%. Data are presented as mean±SD, N=3, n=3, from three independent experiments performed in triplicate.

FIG. 3B presents a schematic presentation of the identified 16 hit compounds according to their chemical skeleton (FIG. 3B).

FIGS. 4A-F present comparative plots showing dose-response curves of the validated hits in PD-1/PD-L1 inhibition (8 doses in 1:2 and 1:10 serial dilutions) starting from 100 μM, using BMS202 (dark gray) as a positive control for PD-1/PD-L1 inhibition. Results were determined by HTRF and are from three independent experiments performed in triplicate.

FIGS. 5A-C present comparative analyses of the inhibitory activity of BMS202 and the tested exemplary small-molecule compounds. FIGS. 5A-B present comparative plots (FIG. 5A and a bar graph (FIG. 5B) showing thermal shifts that indicate the stabilization of PD-L1 by compounds 5 (black), 42 (green), 47 (yellow), 69 (yellow), 75 (black) and 84 (black). FIG. 5A presents curves representing the fraction of unfolded recombinant human PD-L1 protein, where 0 represents the folded PD-L1 and 1 the unfolded, in the presence of 1% DMSO (light gray), indicated compounds (green, yellow and white) and BMS202 (dark gray) at 100 μM. FIG. 5B presents the shift of the melting curve in the presence of the tested compounds (100 PM), quantified by the change in melting temperature (ΔTm), which suggests increased thermal stability of the protein-compound complex. Data are presented as mean±SD, N=2, n=3, from two independent experiments performed in triplicate. FIG. 5C presents comparative NMR spectra showing waterLOGSY NMR experiment of 150 μM BMS202 and Compound 69 in the absence and presence of 5 μM PD-L1.

FIG. 6 presents bar graphs showing the effect of hit compounds on cell viability. Different cell lines were incubated with increased concentrations of the tested compounds for 48 hours. Cell viability was normalized to untreated cells. Three different concentrations 100 μM (blue), 10 μM (green) and 1 μM (gray) were tested. Data are presented as mean±SD, N=3, n=3 and N=1, n=3, from three or one independent experiment(s) performed in triplicate.

FIGS. 7A-D present data obtained in in vitro studies of the inhibition of PD-1/PD-L1 interaction by exemplary compounds of the present embodiments. FIG. 7A show data obtained upon stimulating cell-surface PD-L1 by IFN-γ. Cell-surface PD-L1 was determined by flow cytometry. Cells were stimulated with 200 ng/ml IFN-γ (blue) for 18 hours or left untreated (gray). Bars indicate the percentage of PD-L1 on cells. Data are presented as mean±SD, N=1, n=3, from one independent experiment performed in triplicate. FIG. 7B are bar graphs showing preferential PD-1/PD-L1 inhibition upon co-incubation with exemplary compounds of the present embodiments. MDA-MB-231 (ATCC® HTB-26™) cells (gray) were treated with 10 μM of compounds (green, yellow and black) and anti-PD-L1 (red) for 24, 48, and 72 hours. Data are presented as mean±SD, N=1, n=3, from one independent experiment performed in triplicate. Statistical analysis: one-way ANOVA and Tukey's post-test. FIGS. 7C-D are bar graphs showing inhibition of PD-1/PD-L1 interaction using the breast cancer MDA-MB-231 (FIG. 7C) and melanoma A375 (FIG. 7D) cell lines. 10 μM of compounds (green, yellow, and black), anti-PD-L1 (αPD-L1; red) and BMS202 (dark gray) were incubated with the targeted cells for 72 hours. A375 cells were stimulated with 200 ng/ml IFN-γ (gray) for 18 hours before treatments to enhance PD-L1 levels. The remaining accessible PD-L1 was determined by flow cytometry. Data are presented as mean±SD, N=3, n=9, or N=1, n=3, from three or one independent experiment performed in triplicate. Statistical analysis: one-way ANOVA and Tukey's post-test.

FIGS. 8A-F present data obtained for induction of T-cell activation by PD-1/PD-L1 inhibition using co-culture experiments. FIG. 8A is a schematic representation of the experimental workflow. FIGS. 8B and 8C are bar graphs showing cell-surface PD-L1 levels as determined by flow cytometry. Data in FIG. 8B indicate mean fluorescence intensity (MFI) of anti-PD-L1-BV711 minus MFI of isotype control. FIG. 8D presents representative flow cytometry plots for T-cell reactivity after 72 hours of co-culture with autologous tumor cells. The plots indicate the percentage of IFN-γ and CD107a on CD8⁺ T cells. FIGS. 8E and 8F are bar graphs showing quantification of tumor cells-induced IFN-γ production (FIG. 8E) and CD107a cell-surface expression (FIG. 8F) of CD45⁺CD3⁺CD8⁺ T cells, obtained after 72 hours of co-culture. In FIGS. 8B, 8C, 8E and 8F Tumor cells were obtained from surgical resections of melanoma (Mel), bone metastasis of breast (BBM) and lung cancer (LBM). Cells were stimulated with CD28 (blue), treated with αPD-L1 blocking antibody (red) or small-molecule inhibitor Compound 69 (yellow), or left untreated (gray). Data are presented as mean±SD, N=1, n=3, from one independent experiment performed in triplicate or duplicate (limited amounts of tumor or blood available).

FIG. 9A present data showing CD8⁺ T cell infiltration on 3D melanoma spheroids upon co-culture of 3D tumor spheroids of cells obtained from surgical resection of melanoma (Mel4) and PBMC and grown together in reduced growth factor Matrigel. The spheres and PBMC were either not treated or treated with anti-PD-L1 (αPD-L1) or small-molecule inhibitor Compound 69. The CD8⁺ T-cell infiltration (green) was evaluated after 72 hours of co-culture by confocal microscopy. Scale bar=100 μm.

FIG. 9B shows 3D tumor spheroids sprouting from melanoma patient 4 (Mel4). Treatment with the small-molecule inhibitor Compound 69 inhibited the melanoma cells sprouting. Scale bar=400 μm.

FIGS. 10A-C present in vivo data showing PD-1/PD-L1 small-molecule inhibitor recruits cytotoxic CD8 T cells into the TME. FIG. 10A shows the treatment course as a timeline (days) of tumor inoculation and treatments. FIG. 10B shows comparative plots presenting tumor growth curve of PD-1 humanized mice implanted with the colorectal cancer cell line MC38 expressing humanized PD-L1. Animals were treated by 10 mg/kg intraperitoneal injections, ten daily doses at days 12-21 with small-molecule inhibitor Compound 69 (yellow), or alternatively treated with 5% (v/v) DMSO, 30% (v/v) polyethylene glycol (PEG) 300, 5% (v/v) Tween® 80, and double distilled (dd) H₂O as vehicle control (gray). FIG. 10C shows representative tumor images of each treatment group (vehicle control, atezolizumab, and small-molecule inhibitor Compound 69).

FIGS. 11A-D present bar graphs showing quantification of tumor-infiltrating lymphocytes (FIGS. 11A and 11B) and PD-1/PD-L1 (FIGS. 11C and 11D) following treatment with small-molecule inhibitor Compound 69 (yellow), atezolizumab (red) and vehicle control (gray). Tumor cells were isolated on day 30 after the tumor inoculation and quantification was performed by flow cytometry. Data are presented as mean±standard deviation, N=3 animals. Statistical analysis: one-way ANOVA and Tukey's post-test.

### DESCRIPTION OF SPECIFIC EMBODIMENTS OF THE INVENTION

The present invention, in some embodiments thereof, relates to therapy and, more particularly, but not exclusively, to small molecule compounds which are capable of interfering with PD-1/PD-L1 interaction, and which are usable in treating conditions associated with PD-1 and/or PD-L1 function and/or which are treatable by enhancing T-cell function, such as cancer, neurodegenerative diseases and infectious diseases.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

As delineated in the Background section hereinabove, small-molecule inhibitors constitute promising alternative approaches to therapeutically target immune checkpoint receptors, by offering several unique advantages over monoclonal antibody (mAb) drugs. Small molecules can provide increased oral bioavailability, bio-efficiency, and short half-life activity, which is particularly important for autoimmune or adverse immune events. Small molecules can also offer a greater diffusion rate within the tumor microenvironment (TME), and the possibility of avoiding the macrophage-mediated resistance observed in anti-PD-1 therapy.

Small molecules are technically difficult to identify and assess. Together with a challenging design, the limited structural elucidation of the targets has been compromising the development of PD-1/PD-L1 small-molecule inhibitors. Before 2015, no human PD-1/PD-L1 X-ray structure was resolved, and the murine form does not allow the assessment of the extent of plasticity or interactions established with the PD-L1. In the last years, several human PD-1 and PD-L1 X-ray structures have been resolved to expose the murine/human structural differences within the binding modes between proteins, as well as the plasticity in the complex formation.

However, the design of inhibitors directly targeting the PD-1/PD-L1 interaction interface has been limited by the larger, hydrophobic, and flat interface between proteins without deep binding pockets. Recently, different X-ray structures of PD-L1 with a class of small-molecule inhibitors have been resolved. Bristol Myers Squibb (BMS) compounds were the first non-peptide-based compounds able to inhibit PD-1/PD-L1 interaction, however, they are reported as compounds with poor drug-like properties. In general, these inhibitors bind to PD-L1 leading to a deep cylindrical, hydrophobic pocket created by the interface of two monomers [Zak, K. M. et al. 2016, supra].

These structures served as a starting point for a rational structure-based drug design approach underlying at least some embodiments of the present invention.

The present inventors have established multi-cellular 2D and 3D cancer models using pairs of tumor and peripheral blood mononuclear cells (PBMC) isolated from patients, and have used these models to identify PD-1/PD-L1 small-molecule inhibitors that may present unique advantages over monoclonal antibodies (mAb) currently used in the clinic. These studies led to the identification of novel small molecules able to promote T-cell activation by targeting the PD-1/PD-L1 co-inhibitory interactions, which proved to be as effective as mAb. Unlike biological compounds, the newly-identified small molecules enabled an extensive infiltration of T lymphocytes into the 3D solid cancer models, unveiling a unique potential to transform cancer immunotherapy. This added value in terms of tumor-infiltrating effector T cells was corroborated in vivo in a colorectal cancer model expressing the human PD-1 on T cells and the human PD-L1 on tumor cells.

Some embodiments of the present invention relate to the identification and validation of PD-1/PD-L1 small-molecule inhibitors with enhanced therapeutic properties that offer the possibility to circumvent the inherent challenges associated with mAb.

As described in the Examples section that follows, the present inventors have followed a trans-disciplinary approach to discover novel small molecules that can modulate PD-1/PD-L1 interaction. To that end, the present inventors have employed in silico analyses combined with in vitro, ex vivo and in vivo experimental studies to assess the ability of novel compounds to modulate PD-1/PD-L1 interaction and enhance T-cell function.

More specifically, the present inventors have developed a computationally-driven approach to identify small-molecule inhibitor candidates (hit compounds), where nearly 900,000 compounds from synthetic compound libraries were screened through a structure-based virtual screening campaign. Biochemical experiments have confirmed and validated in silico candidates as true inhibitors of PD-1/PD-L1 interactions. These results were subsequently corroborated in vitro. The impact of the uncovered small molecules (compounds) on T-cell function was performed by exploiting newly-developed 2D and 3D multi-cellular cancer models using patient-derived peripheral blood mononuclear cells (PBMC) and autologous tumor cells. These studies led to the identification of new small molecules that restore T-cell function and enable their extensive infiltration into 3D tumor spheroids by targeting the PD-1/PD-L1 co-inhibitory interactions and has proven to be a powerful strategy for drug discovery in the cancer immunotherapy field.

These studies identified small molecule PD-1/PD-L1 candidates that reached the same level of PD-1/PD-L1 inhibition as the mAb PD-L1. Surprisingly, the small-molecule inhibitor showed higher secretion of IFN-7, CD107a upregulation, and consequently an enhanced T-cell activation, compared to the α-PD-L1. Overall, reproducible responses were observed among the different analyses employing distinct cancer patient/PBMC paired samples. Therefore, considering the potential enhanced bioavailability and diffusion through the tumor microenvironment, in addition to the demonstrated highly improved infiltration of activated T cell into tumor mass in vivo, these small molecules targeting the PD-1/PD-L1 interaction present unique advantages over mAb currently in the clinic.

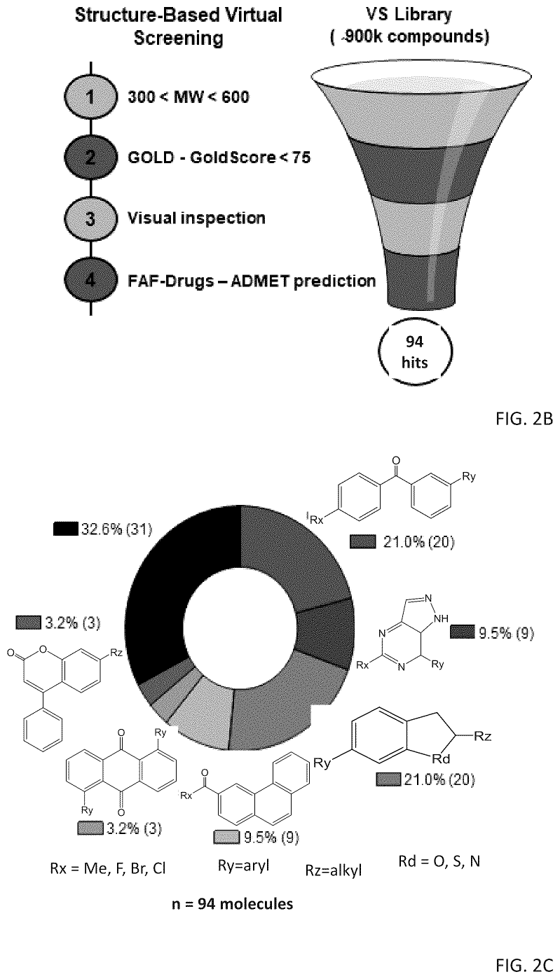

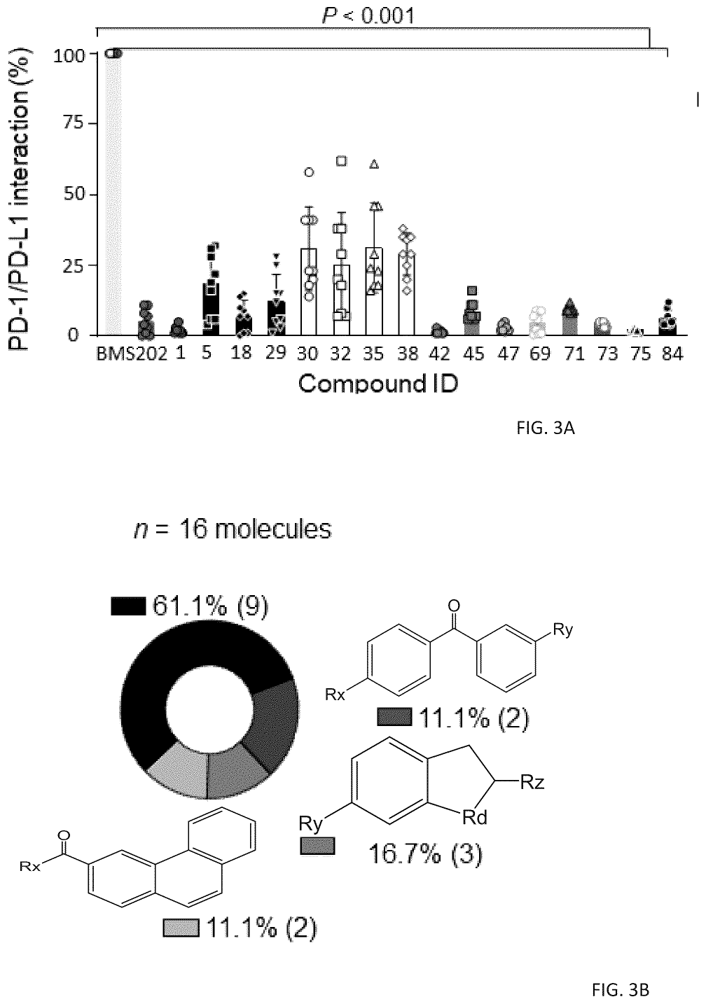

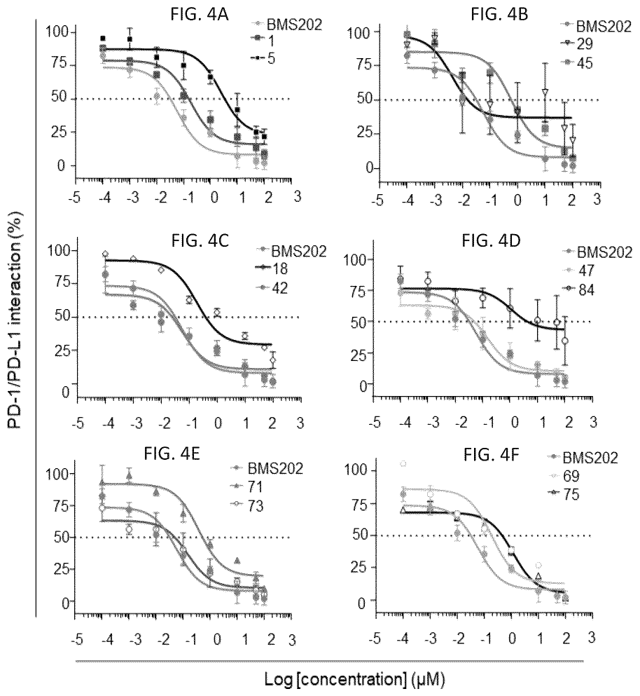

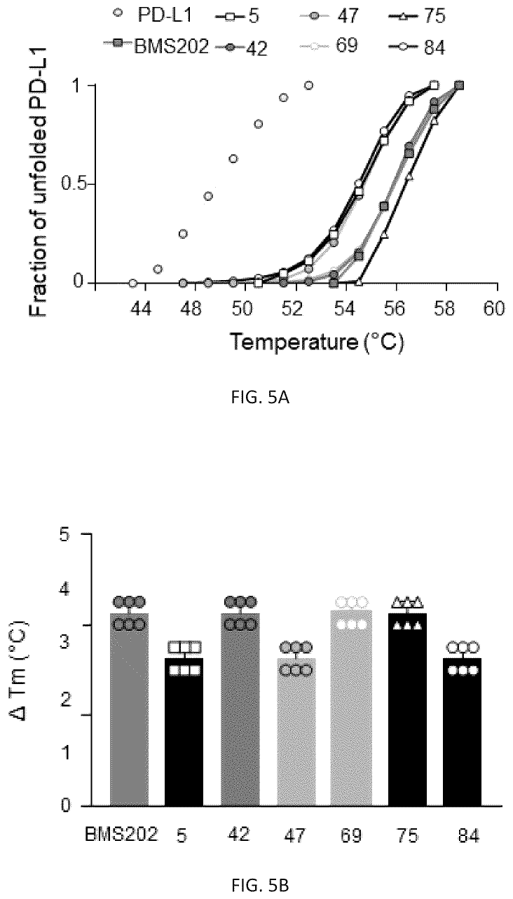

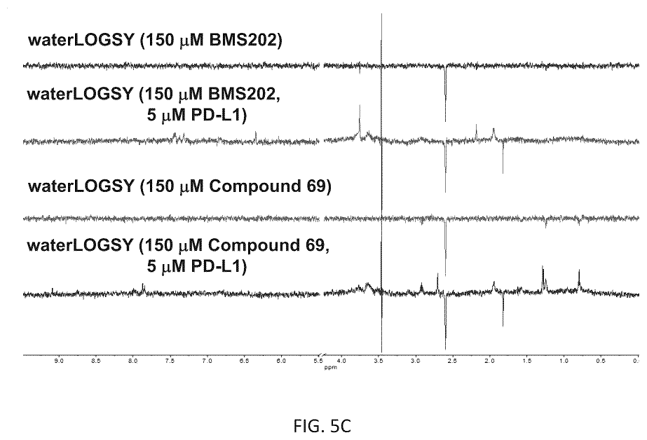

More specifically, in silico studies (structure-based virtual screening using molecular docking) led to the selection of 94 virtual hits presenting good spatial fitting within the PD-L1 pocket, high score values, key interaction to pocket residues, as well as, good ADMET properties (see, FIGS. 2B, 2C, and Table A in Example 1). The hit validation achieved 16 (17%) compounds using a standard biochemical fluorescence-based PD-1/PD-L1 binding assay (see, FIGS. 3A, 4A-F and Table B in Example 1). The hits identified displayed mainly phenanthrene (2 validated hits out of 9 initial hits), benzo[d]thiazole (3 validated hits out of 20 initial hits) and benzophenone scaffolds (see, FIGS. 3A and 3B). The validated hits were shown to bind to PD-L1 as BMS inhibitors, as confirmed by DSF binding studies (see, FIGS. 5A-B). This binding to PD-L1 was further confirmed by a WaterLOGSY NMR assay (see, FIG. 5C).

While moving forward in the discovery of new small-molecule inhibitors towards the characterization of their biological effect, the present inventors have realized that the type of assays already developed to validate the effect of PD-1/PD-L1 small-molecule inhibitors are highly limited. These experiments use the biochemical assays employed for the hit validation and/or engineered cell-based assays. The present inventors have therefore decided to evaluate compounds activity using innovative and non-engineered 2D and 3D cell-based assays. Initially, studies were focused on PD-1/PD-L1 inhibition, and for that, different cell lines were selected. Two types of cancer cells, breast cancer, and melanoma cell lines were thus selected to perform the in vitro studies looking at the impact of the hit compounds on PD-L1/PD-1 interaction. The basis for cell line selection was the remarkable results on highly immunogenic tumors, as melanoma, and the exciting outcomes in the treatment of other tumors reported as poorly immunogenic, as breast cancer.

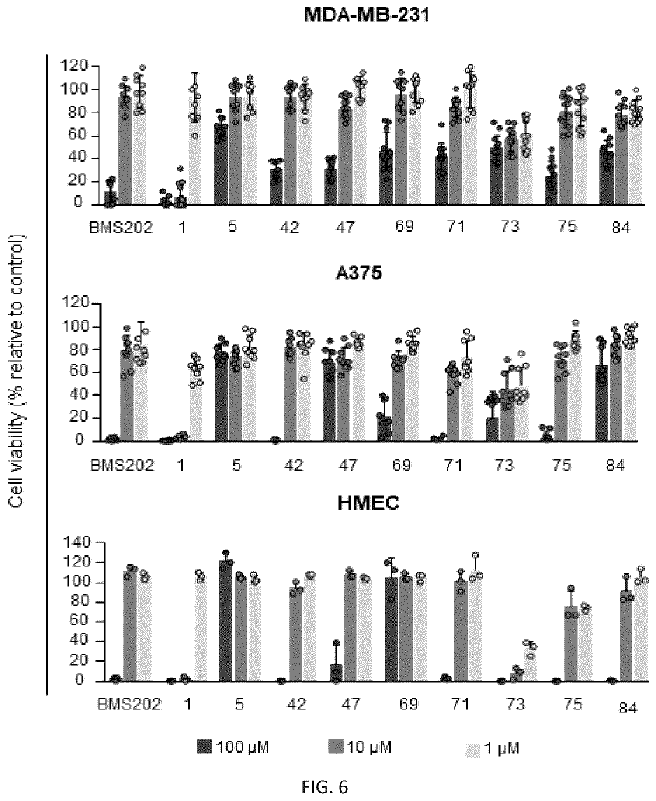

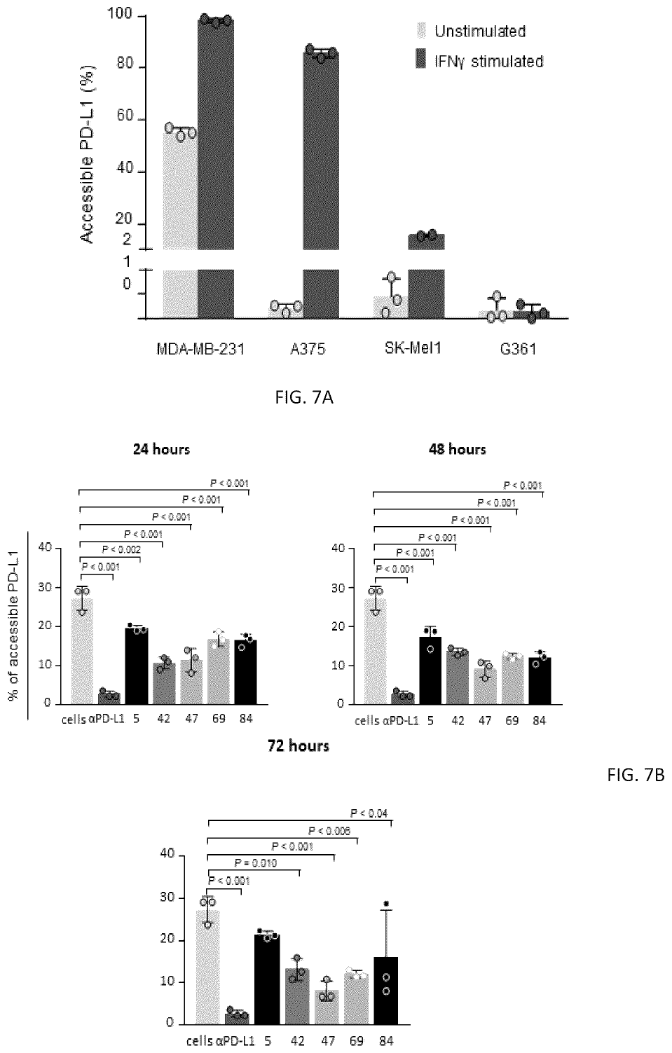

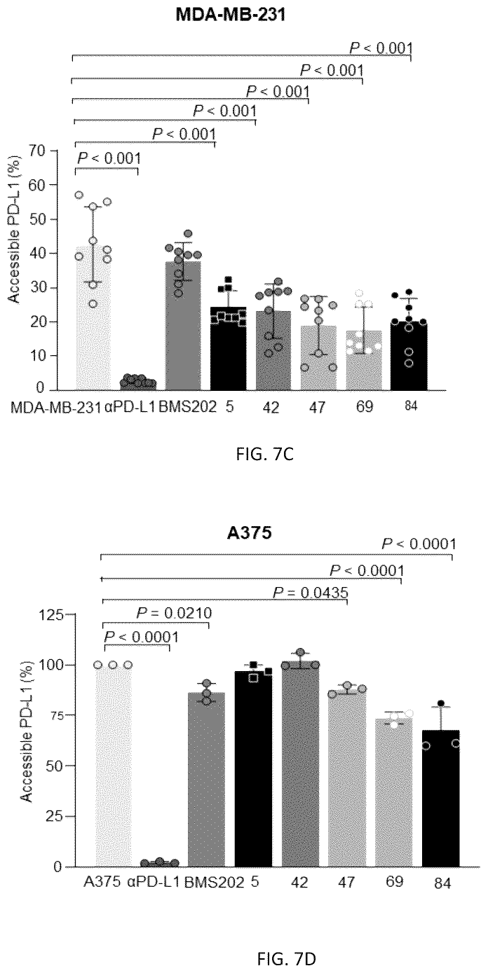

Out of 16 uncovered hit compounds, the compounds that presented a lower impact on cell viability were favorable considered as suitable for their further characterization as potential PD-1/PD-L1 inhibitors in vitro and ex vivo (e.g., compounds 5, 42, 47, 69 and 84; see, FIGS. 6, 7A-D and Table B in Example 1).

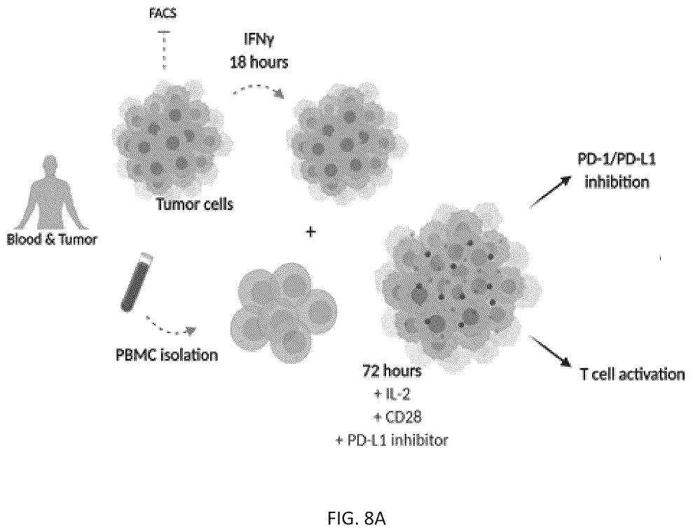

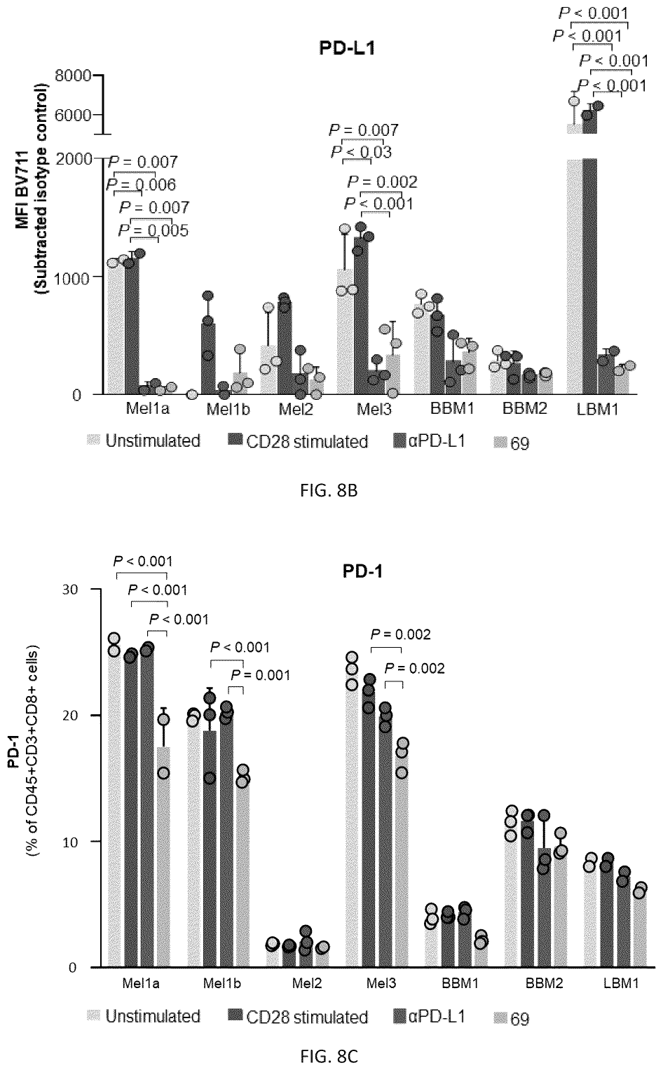

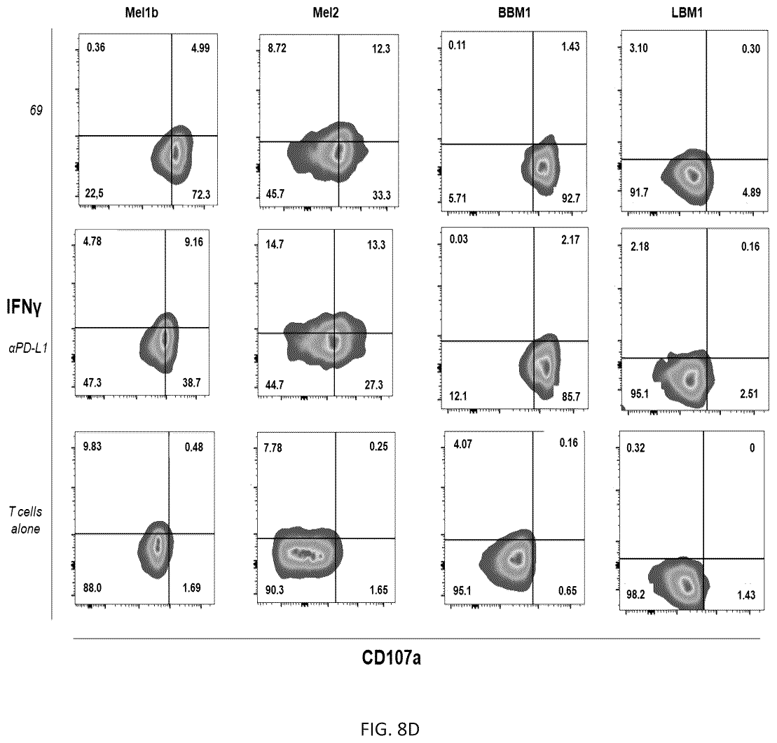

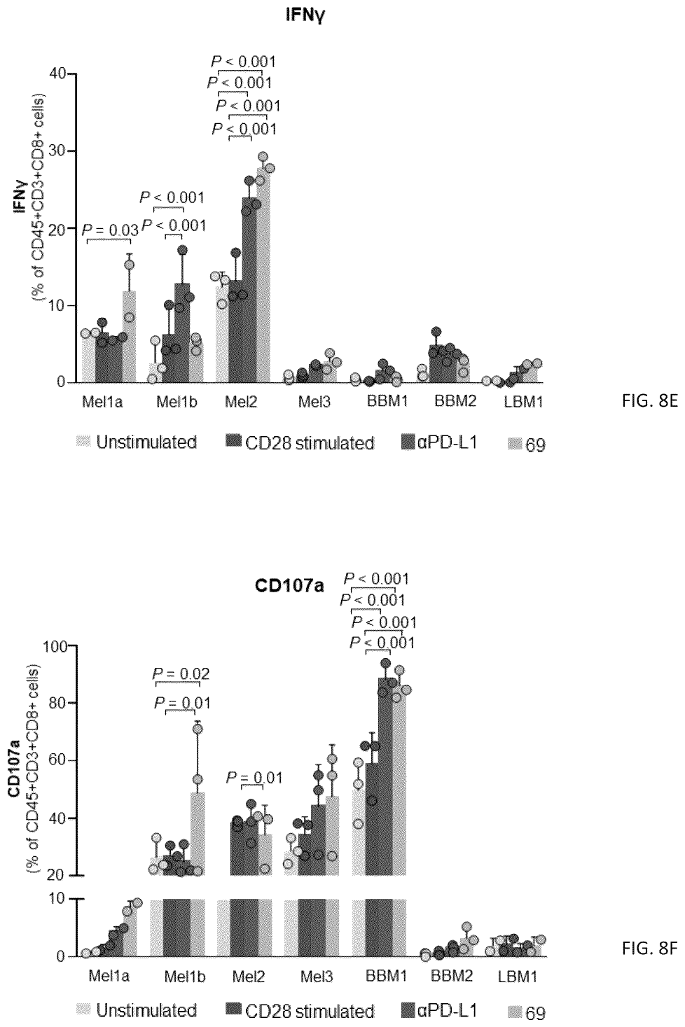

The ultimate role of an exemplary small-molecule inhibitor (Compound 69) was assessed in T-cell activation using 2D and 3D co-culture studies of paired matched patient-derived tumor cells and PBMC. Only tumor cells and PBMC of the same patient were co-cultured to ensure that an HLA-mismatch reaction did not occur, and unspecific T-cell activation could be observed. In contrast to tumor cell lines, patient-specific model systems are proving a most valuable tool in the field of immune-oncology due to the inherent diversity of the disease and the multifactorial nature of T cell-mediated tumor destruction. In this assay, it was possible to provide a proof of concept that patient samples obtained from tumor resection of melanoma, breast and lung cancer metastasis at surgery treated with the most promising PD-1/PD-L1 inhibitor could activate T cells by inhibiting this pathway (see, FIGS. 8A-F and 9A-B).

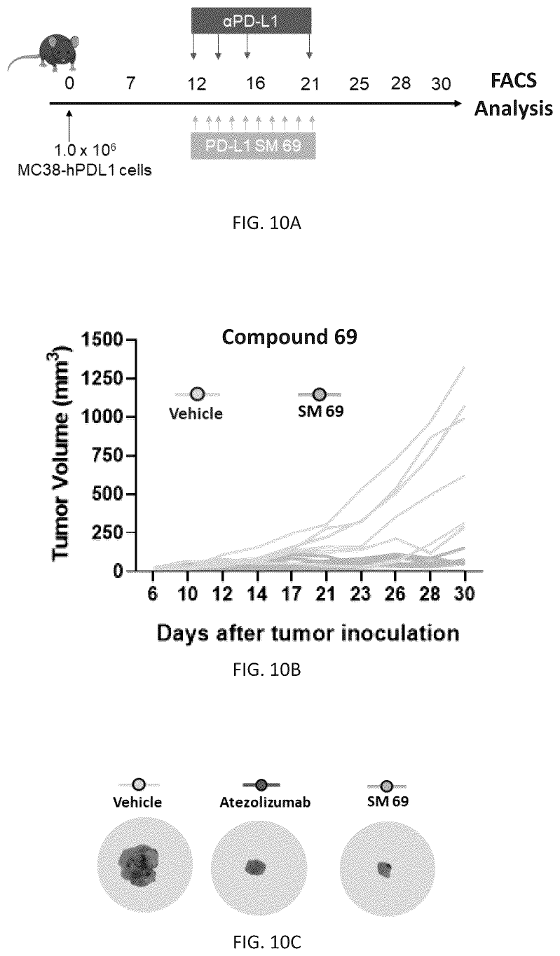

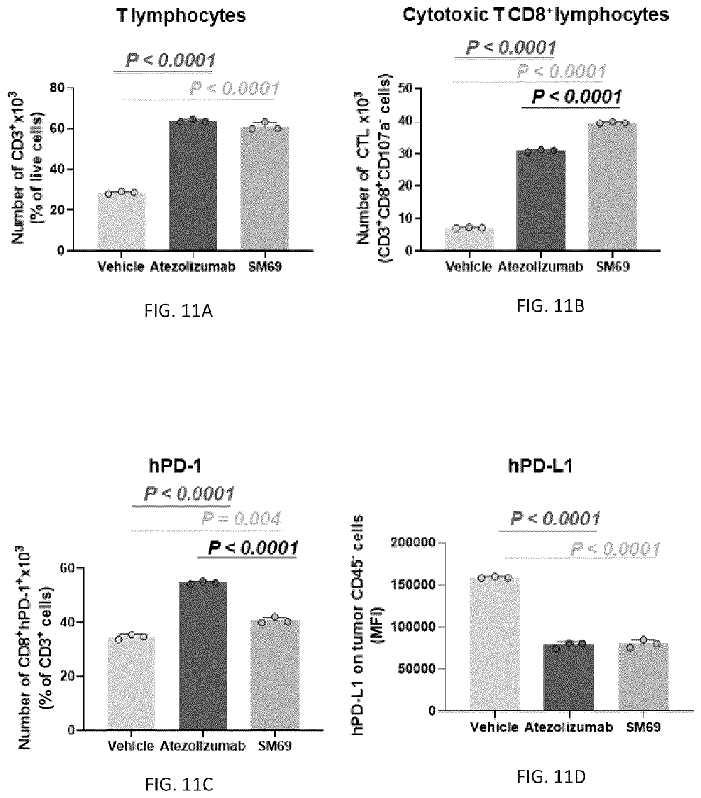

The exemplary small-molecule inhibitor Compound 69 was tested in in vivo experiments while comparing its activity to that of a clinically relevant αPD-L1 antibody. Compound 69 reduced the tumor volume by 93.6% when compared with vehicle control (see, FIGS. 10A-C) and significantly outperformed the monoclonal antibody in recruiting cytotoxic T-cells into the TME (see, FIGS. 11A-D).

The studies that led to the present invention resulted in PD-L1/PD-1 targeting small-molecules that are usable in the treatment of cancer, particularly cancers overexpressing PD-L1/PD-1 (e.g. lung cancer, melanoma, breast cancer, renal cancer, bladder cancer).

Embodiments of the present invention therefore relate to small-molecule compounds for use in treating cancer, and/or in interfering with a PD-L1-PD1 interaction, and/or in enhancing T-cell function.

Compounds:

Excluded from the scope of the present embodiments are small molecule compounds known as BMS compounds, and compounds such as described in Abdel-Magid, A. F. ACS Med. Chem. Lett. 6, 489-490 (2015); Guzik, K. et al. J. Med. Chem. 1, acs.jmedchem.7b00293 (2017); Skalniak, L. et al. Oncotarget 8, 72167-72181 (2017); Acnrcio, R. C. et al. Medchemcomm 10, 1810-1818 (2019); Zak, K. M. et al. Structure 23, 2341-2348 (2015); Zak, K. M. et al. Oncotarget 7, 30323-30335 (2016); Qin, M. et al. J. Med. Chem. 62, 4703-4715 (2019); Blevins, D. J. et al. ACS Med. Chem. Lett. 10, 1187-1192 (2019); Acúrcio, R. C. et al, J. Med. Chem. 61, 10957-10975 (2018); and Acnrcio, R. C. et al., WIREs Comput Mol Sci. 9, e1397 (2018).

According to an aspect of some embodiments of the present invention, the compounds are collectively represented by Formula I:

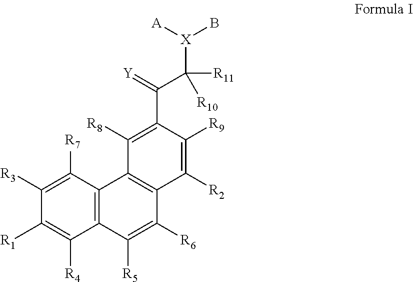

or a pharmaceutically acceptable salt thereof,
wherein:
   R₁-R₁₁ are each independently selected from hydrogen, alkyl, cycloalkyl, aryl, heteroaryl, heteroalicyclic, alkoxy, aryloxy, hydroxy, thiol, thioalkoxy, thioaryloxy, amine, imine, halo, nitro, nitrile (cyano), amide, hydrazine, hydrazide, carboxylate, thiocarboxylate, carbamate, thiocarbamate, sulfonyl, sulfinyl, sulfonamide, carbonate, thiocarbonate, sulfoxide, thiosulfate, sulfate, sulfite, thiosulfite, phosphonate, urea, thiourea, guanyl and guanidyl;
   Y is O or S;
   X is O, S or N, wherein when X is O or S, B is absent; and
   A and B (if present) are each independently selected from hydrogen, alkyl, cycloalkyl, aryl, heteroaryl, and heteroalicyclic, the alkyl, cycloalkyl, aryl, heteroaryl, and heteroalicyclic being independently substituted (e.g., as described herein) or unsubstituted.

Exemplary such compounds are referred to herein as Compounds 47 and 69 (see, Table B).

According to some of any of the embodiments described herein for Formula I, R₁ and R₂ are each independently selected from hydrogen, alkyl and cycloalkyl, or from hydrogen and alkyl.

According to some of any of the embodiments described herein for Formula I, at least one of R₁-R₉ is alkyl. According to some embodiments, the alkyl is, independently (in case two or more of R₁ to R₉ is an alkyl) a lower alkyl of 1 to 10, or of 1 to 6, carbon atoms. The alkyl can be methyl, ethyl, propyl isopropyl, butyl, isobytyl, tert-butyl, pentyl, hexyl, etc.

According to some of any of the embodiments described herein for Formula I, R₁ is an alkyl, e.g., as described herein. In exemplary embodiments, R₁ is isopropyl. Alternatively, R₁ is hydrogen.

According to some of any of the embodiments described herein for Formula I, R₂ is alkyl, e.g., as described herein. In exemplary embodiments, R₂ is methyl. Alternatively, R₂ is hydrogen.

According to some of any of the embodiments described herein for Formula I, one of R₁-R₉ is alkyl and the other substituents are each hydrogen.

According to some of any of the embodiments described herein for Formula I, two of R₁-R₉ is alkyl and the other substituents are each hydrogen. According to some of any of the embodiments described herein for Formula I, one of R₁-R₉ is ispropyl and the other substituents are each hydrogen.

According to some of any of the embodiments described herein for Formula I, one of R₁, R₃, R₄ and R₆ is alkyl as described herein, for example, isopropyl. According to some of these embodiments, the other substituents are each hydrogen. Alternatively, one of R₂, R₅ and R₇-R₉ is an alkyl, e.g., as described herein.

According to some of any of the embodiments described herein for Formula I, one of R₁-R₉ is methyl and the other substituents are each hydrogen.

According to some of any of the embodiments described herein for Formula I, one of R₂, R₅ and R₇-R₉ is an alkyl, e.g., as described herein, for example, methyl. According to some of these embodiments, the other substituents are each hydrogen, or one or more of R₁, R₃, R₄ and R₆ is alkyl as described herein, for example, isopropyl.

According to some of any of the embodiments described herein for Formula I, R₁₀ and R₁₁ are each independently selected from hydrogen and alkyl. The alkyl is preferably a lower alkyl as described herein.

According to some of any of the embodiments described herein for Formula I, R₁₀ and R₁₁ are each hydrogen.

According to some of any of the embodiments described herein for Formula I, R₁₀ and R₁₁ are each independently a lower alkyl as described herein.

According to some of any of the embodiments described herein for Formula I, one of R₁₀ and R₁₁ is an alkyl, e.g., a lower alkyl as described herein, for example, methyl, and the other is hydrogen.

According to some of any of the embodiments described herein for Formula I, R₁, R₂ and R₁₁ are each independently an alkyl, e.g., a lower alkyl as described herein, and R₁₀ is hydrogen.

According to some of any of the embodiments described hereinabove for Formula I, X is N.

According to some of any of the embodiments described herein for Formula I, A and B (if present) are each independently an alkyl. Alternatively, one or each of A and B can independently be an alkyl and/or cycloalkyl. According to some of these embodiments, X is N.

According to some of any of the embodiments described herein for Formula I, A is an alkyl and B is hydrogen, an alkyl or a cycloalkyl. According to some of these embodiments, X is N.

According to some of any of the embodiments described herein for Formula I, A is a cycloalkyl and B is hydrogen, an alkyl or a cycloalkyl. According to some of these embodiments, X is N.

According to some of any of the embodiments described herein for Formula I, A and B are each hydrogen. According to some of these embodiments, X is N.

According to some of any of the embodiments described herein for Formula I, X is N, and A and B are each independently an alkyl, preferably an alkyl of 1-10, or of 1-8, or of 2-10, or of 2-8, or of 2-6, carbon atoms in length. According to some of these embodiments, the alkyl is a linear alkyl. According to exemplary embodiments, X is N and A and B are each pentyl.

According to some of any of the embodiments described herein for Formula I, R₃-R₁₀ are each hydrogen.

According to some of any of the embodiments described herein for Formula I, X is N, and A and B are each independently an alkyl as described herein (e.g., pentyl), and at least one or at least two of R₁-R₉ is/are an alkyl, e.g., a lower alkyl as described herein.

According to some of any of the embodiments described herein for Formula I, X is N, and at least one or at least two of R₁-R₉ is/are an alkyl, e.g., a lower alkyl as described herein.

According to some of any of the embodiments described herein for Formula I, X is N, A and B are each independently an alkyl as described herein (e.g., pentyl), at least one or at least two of R₁-R₉ is/are an alkyl, e.g., a lower alkyl as described herein, and at least one of R₁₀ and R₁₁ is an alkyl, e.g., a lower alkyl as described herein.

According to some of any of the embodiments described herein for Formula I, X is N, at least one or at least two of R₁-R₉ is/are an alkyl, e.g., a lower alkyl as described herein, and at least one of R₁₀ and R₁₁ is an alkyl, e.g., a lower alkyl as described herein.

According to some of any of the embodiments described herein for Formula I, X is N, A and B are each independently an alkyl as described herein (e.g., pentyl), and R₁, R₂ and R₁₁ are each independently an alkyl, e.g., a lower alkyl as described herein. According to some of these embodiments described, R₃-R₁₀ are each hydrogen.

According to some of any of the embodiments described herein for Formula I, X is N, and R₁, R₂ and R₁₁ are each independently an alkyl, e.g., a lower alkyl as described herein. According to some of these embodiments described, R₃-R₁₀ are each hydrogen. According to some of these embodiments described, A and B are each independently an alkyl as described herein (e.g., pentyl).

According to some of any of the embodiments described hereinabove for Formula I, Y is O.

An exemplary compound according to these embodiments is Compound 69 (see, Table B).

According to some of any of the embodiments described herein for Formula I, X is S and B is absent.

According to some of any of the embodiments described herein for Formula I, A is a heterocyclic moiety (a heteroalicyclic or a heteroaryl), and in some embodiments, A is a heteroaryl.

According to some of any of the embodiments described herein for Formula I, X is S, B is absent and A is heterocyclic moiety (a heteroalicyclic or a heteroaryl), preferably a heteroaryl.

When A is a heterocyclic moiety, it can be, for example, a nitrogen-containing, an oxygen-containing, or a sulfur-containing heterocyclic moiety.

According to some of any of the embodiments described herein for Formula I A is an oxygen-containing heterocyclic moiety.

According to some of any of the embodiments described herein for Formula I A is a heteroaryl, and the heteroaryl can be, for example, a nitrogen-containing, an oxygen-containing, or a sulfur-containing, heteroaryl.

According to some of any of the embodiments described herein for Formula I A is an oxygen-containing heteroaryl. In exemplary embodiments, A is pyrylium.

According to some of any of the embodiments described herein for Formula I, R₁-R₁₁ are each hydrogen.

According to some of any of the embodiments described herein for Formula I, X is S, B is absent and A is heterocyclic moiety (a heteroalicyclic or a heteroaryl), preferably a heteroaryl as described herein in any of the respective embodiments, and R₁-R₁₁ are each hydrogen. Alternatively, one or two of R₁-R₁₁ is/are an alkyl, preferably a lower alkyl as described herein.

According to some of any of the embodiments described herein for Formula I, X is S, B is absent and A is an oxygen-containing heterocyclic moiety (a heteroalicyclic or a heteroaryl), preferably an oxygen-containing heteroaryl as described herein in any of the respective embodiments, and R₁-R₁₁ are each hydrogen. Alternatively, one or two of R₁-R₁₁ is/are an alkyl, preferably a lower alkyl as described herein.

According to some of any of the embodiments described hereinabove for Formula I, Y is O.

An exemplary compound according to these embodiments is Compound 47 (see, Table B).

According to an aspect of some embodiments of the present invention, the compounds are collectively represented by Formula II:

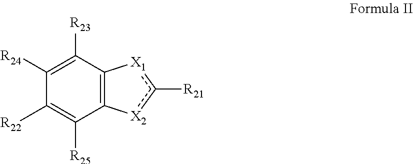

or a pharmaceutically acceptable salt thereof,
wherein:
X₁ and X₂ are each independently selected from N, NR₂₈, S, O, CR₂₆, and CR₂₆R₂₇, at least one of X₁ and X₂ being N, NR₂₈, S or O, wherein each of the dashed lines represents an optional bond (forming a double bond) when the adjacent X₁ or X₂ is N or CR₂₆;
R₂₈ is hydrogen, alkyl, cycloalkyl or aryl; and
R₂₁-R₂₇ are each independently selected from hydrogen, alkyl, cycloalkyl, aryl, hydroxy, alkoxy, aryloxy, thiol, thioalkoxy, thioaryloxy, amine, imine, halo, nitrile (cyano), nitro, amide, hydrazine, hydrazide, carboxylate, thiocarboxylate, carbamate, thiocarbamate, sulfonyl, sulfinyl, sulfonamide, carbonate, thiocarbonate, sulfoxide, thiosulfate, sulfate, sulfite, thiosulfite, phosphonate, urea, thiourea, guanyl and guanidyl.

According to some of any of the embodiments described herein for Formula II, at least one or at least two of R₂₁-R₂₅ is a heteroatom-containing moiety as described herein.

According to some of any of the embodiments described herein for Formula II, at least one and preferably both of R₂₁ and R₂₂ is a heteroatom-containing moiety as described herein, which can be the same or different, and is preferably different.

According to the present embodiments, a heteroatom-containing moiety encompasses any substituent that features one or more heteroatom(s) such as oxygen, nitrogen and/or sulfur. Exemplary substituents include, but are not limited to, alkoxy, aryloxy, thiol, thioalkoxy, thioaryloxy, amine, imine, nitrile (cyano), amide, hydrazine, hydrazide, carboxylate, thiocarboxylate, carbamate, thiocarbamate, sulfonyl, sulfinyl, and sulfonamide.

Exemplary compounds according to this aspect of the present embodiments are compounds 42 and 71 (see, Table B).

According to some of any of the embodiments described herein for Formula II, X₁ is N such that the adjacent bond is a double bond (the dashed line denotes a double bond from X₁ to the indicated adjacent carbon) or X is NR₂₈ such that the dashed line is absent and the adjacent bond is a single bond (the dashed line denotes a single bond from X₁ to the indicated adjacent carbon).

According to some of any of the embodiments described herein for Formula II, X₁ is N (such that the adjacent bond is a double bond).

According to some of any of the embodiments described herein for Formula II, X₂ is S, such that the dashed line is absent and the adjacent bond is a single bond (the dashed line denotes a single bond from X₂ to the indicated adjacent carbon).

According to some of any of the embodiments described herein for Formula II, X₁ is N (such that the adjacent bond is a double bond) and X₂ is S, such that the dashed line is absent and the adjacent bond is a single bond (the dashed line denotes a single bond from X₂ to the indicated adjacent carbon).

According to some of any of the embodiments described herein for Formula II, one or both of R₂₁ and R₂₂ is a sulfur-containing moiety, such as, for example, a thioalkoxy, a thioaryloxy, a thiocarboxylate, a thiocarbamate, sulfonyl, sulfinyl, or sulfonamide, each can be substituted or unsubstituted, as defined herein.

According to some of any of the embodiments described herein for Formula II, one or both of R₂₁ and R₂₂ is a thioalkoxy, as defined herein.

According to some embodiments, the thioalkoxy comprises an alkyl of from 1 to 20, or from 1 to 12, or from 1 to 10, or from 2 to 20, or from 2 to 12, or from 2 to 10, or from 5 to 20, or from 5 to 12, or from 5 to 10, or of at least 4, at least 5, at least 6, at least 7, or at least 8, carbon atoms in length.

In exemplary embodiments, the thioalkoxy is a thiooctyl.

According to some of any of the embodiments described herein for Formula II, X₁ is N (such that the adjacent bond is a double bond), X₂ is S, such that the dashed line is absent and the adjacent bond is a single bond (the dashed line denotes a single bond from X₂ to the indicated adjacent carbon), and R₂₁ is a sulfur-containing moiety as described herein.

According to some of any of the embodiments described herein for Formula II, X₁ is N (such that the adjacent bond is a double bond), X₂ is S, such that the dashed line is absent and the adjacent bond is a single bond (the dashed line denotes a single bond from X₂ to the indicated adjacent carbon), and R₂₁ is a thioalkoxy as described herein.

According to some of any of the embodiments described herein for Formula II, R₂₂ is a nitrogen-containing moiety.

According to some of any of the embodiments described herein for Formula II, X₁ is N (such that the adjacent bond is a double bond), X₂ is S, such that the dashed line is absent and the adjacent bond is a single bond (the dashed line denotes a single bond from X₂ to the indicated adjacent carbon), R₂₁ is a sulfur-containing moiety as described herein, and R₂₂ is a nitrogen-containing moiety, as described herein.

A nitrogen-containing moiety can be, for example, amine, imine, nitrile (cyano), amide, hydrazine, hydrazide, carbamate, thiocarbamate, and sulfonamide, each can be substituted or unsubstituted, as defined herein.

According to some of any of the embodiments described herein for Formula II, R₂₂ is an imine (—N═CR′R″, wherein R′ and R″ are as defined herein).

According to some of any of the embodiments described herein for Formula II, R₂₂ is a substituted imine (—N═CR′R″, wherein at least one of R′ and R″ is other than hydrogen).

According to some of these embodiments, the imine is substituted by one or more of alkyl, cycloalkyl, heteroalicyclic, aryl, and heteroaryl, and in some embodiments the imine is substituted by an aryl such as phenyl, which can be substituted or unsubstituted. In exemplary embodiments, the imine is substituted by phenol.

According to some of any of the embodiments described herein for Formula II, X₁ is N (such that the adjacent bond is a double bond), X₂ is S, such that the dashed line is absent and the adjacent bond is a single bond (the dashed line denotes a single bond from X₂ to the indicated adjacent carbon), R₂₁ is a sulfur-containing moiety as described herein, and R₂₂ is an imine, as described herein. According to some of these embodiments, R₂₂ is an imine substituted by an aryl (e.g., phenol).

According to some of any of the embodiments described herein for Formula II, X₁ is N (such that the adjacent bond is a double bond), X₂ is S, such that the dashed line is absent and the adjacent bond is a single bond (the dashed line denotes a single bond from X₂ to the indicated adjacent carbon), R₂₁ is a thioalkoxy as described herein, and R₂₂ is an imine, as described herein. According to some of these embodiments, R₂₂ is an imine substituted by an aryl (e.g., phenol).

According to some of any of the embodiments described herein for Formula II, R₂₃₋₂₅ are each hydrogen.

An exemplary compound according to these embodiments is Compound 42 (see, Table B).

According to some of any of the embodiments described herein for Formula II, X₁ is NR₂₈ such that the dashed line is absent and the adjacent bond is a single bond (the dashed line denotes a single bond from X₁ to the indicated adjacent carbon), and X₂ is CR₂₆ such that the respective dashed line denotes a double bond (the dashed line denotes a denotes a double bond from X₂ to the indicated adjacent carbon).

According to some of these embodiments, R₂₈ and R₂₆ are each hydrogen. Alternatively, one or both of R₂₈ and R₂₆ can be other than hydrogen. For example, R₂₈ can be alkyl, cycloalkyl or aryl. For example, R₂₆ can be alkyl, alkoxy, halo, amine, hydroxy, thiohydroxy, thioalkoxy, etc.

According to some of any of the embodiments described herein for Formula II, one or both of R₂₁ and R₂₂ is a nitrogen-containing moiety as described herein. In some embodiments, the nitrogen-containing moiety is hydrazide as defined herein (—C(═O)—NR′—NR′R′″). Alternatively, R₂₁ is a thiohydrazide (—C(═S)—NR′—NR′R′″, with R′, R″ and R′″ being as defined herein).

According to some of any of the embodiments described herein for Formula II, X₁ is NR₂₈ such that the dashed line is absent and the adjacent bond is a single bond (the dashed line denotes a single bond from X₁ to the indicated adjacent carbon), X₂ is CR₂₆ such that the respective dashed line denotes a double bond (the dashed line denotes a denotes a double bond from X₂ to the indicated adjacent carbon), and one or both of R₂₁ and R₂₂ is a nitrogen-containing moiety as described herein. According to some of these embodiments, R₂₁ is a nitrogen-containing moiety as described herein.

According to some of any of the embodiments described herein for Formula II, X₁ is NR₂₈ such that the dashed line is absent and the adjacent bond is a single bond (the dashed line denotes a single bond from X₁ to the indicated adjacent carbon), X₂ is CR₂₆ such that the respective dashed line denotes a double bond (the dashed line denotes a denotes a double bond from X₂ to the indicated adjacent carbon), and R₂₁ is a hydrazide as described herein.

According to some of any of the embodiments described herein for Formula II, the hydrazide is substituted, such that at least one of R′, R″ and R′″ is other than hydrogen. According to some of these embodiments, one or both of R″ and R′″ is other than hydrogen, and according to some of these embodiments, one of R′ and R′″ is a glycol-containing moiety. Alternatively, one of R″ and R′″ is an ether, a thioether, alkyl, cycloalkyl, aryl, or any other substituent as described herein.

According to some of any of the embodiments described herein for Formula II, R₂₁ is a nitrogen-containing moiety such as hydrazide (e.g., substituted by a glycol-containing moiety).

According to some of any of the embodiments described herein for Formula II, X₁ is NR₂₈ such that the dashed line is absent and the adjacent bond is a single bond (the dashed line denotes a single bond from X₁ to the indicated adjacent carbon), X₂ is CR₂₆ such that the respective dashed line denotes a double bond (the dashed line denotes a denotes a double bond from X₂ to the indicated adjacent carbon), and R₂₁ is a hydrazide substituted by a glycol-containing moiety.

According to some of any of the embodiments described herein for Formula II, R₂₂ is an oxygen-containing moiety, for example, alkoxy, aryloxy, amide, carboxylate, or carbamate. Alternatively, R₂₂ can be a sulfur-containing moiety as defined herein, for example, thioalkoxy, thioaryloxy, thioamide, thiocarboxylate, or thiocarbamate.

According to some of any of the embodiments described herein for Formula II, R₂₂ is an oxygen-containing moiety.

According to some of any of the embodiments described herein for Formula II, R₂₂ is an alkoxy or a thioalkoxy. According to some of these embodiments, the alkoxy or thioalkoxy is substituted, and the substituent can be as described herein. In exemplary embodiments, the alkoxy or thioalkoxy is substituted by an aryl such as phenyl, and in some embodiments, the alkoxy or thioalkoxy is an alkaryloxy or alkarylthioalkoxy (—OR′ or —SR′, in which R′ is alkaryl such as benzyl).

According to some of any of the embodiments described herein for Formula II, R₂₂ is an alkoxy, for example, alkaryloxy.

According to some of any of the embodiments described herein for Formula II, X₁ is NR₂₈ such that the dashed line is absent and the adjacent bond is a single bond (the dashed line denotes a single bond from X₁ to the indicated adjacent carbon), X₂ is CR₂₆ such that the respective dashed line denotes a double bond (the dashed line denotes a denotes a double bond from X₂ to the indicated adjacent carbon), R₂₁ is a nitrogen-containing moiety as described herein and R₂₂ is an oxygen-containing moiety as described herein.

According to some of any of the embodiments described herein for Formula II, X₁ is NR₂₈ such that the dashed line is absent and the adjacent bond is a single bond (the dashed line denotes a single bond from X₁ to the indicated adjacent carbon), X₂ is CR₂₆ such that the respective dashed line denotes a double bond (the dashed line denotes a denotes a double bond from X₂ to the indicated adjacent carbon), R₂₁ is a hydrazide as described herein and R₂₂ is an alkoxy or thioalkoxy, as described herein. According to some of any of the embodiments described herein for Formula II, each of R₂₃-R₂₈ is hydrogen.

According to some of any of the embodiments described herein for Formula II, each of R₂₃-R₂₅ is hydrogen.

An exemplary compound according to these embodiments is Compound 71 (see, Table B).

According to another aspect of some embodiments of the present invention the compounds are collectively represented by Formula III:

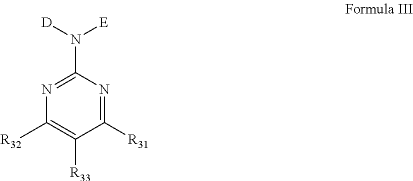

or a pharmaceutically acceptable salt thereof,
wherein:
R₃₃ is hydrogen, alkyl, cycloalkyl, aryl, halo, amine, hydroxy, thiol, aryl, alkoxy, thioalkoxy, aryloxy, thioaryloxy, or, alternatively, forms a cyclic ring with R₃₁ or R₃₂;
R₃₁ and R₃₂ are each independently selected from hydrogen, halo, alkyl, aryl, and amine, or, alternatively, one of R₃₁ and R₃₂ forms a cyclic ring with R₃₃; and D and E are each independently selected from hydrogen, alkyl, cycloalkyl, aryl, heteroaryl, carbonyl (carbonate), thiocarbonyl (thiocarbonate), carboxylate, thiocarboxylate, sulfonyl, sulfinyl, sulfoxide, thiosulfate, sulfate, sulfite, thiosulfite, urea, thiourea, guanyl and guanidyl. According to some of any of the embodiments described herein for Formula III, at least one or at least two of R₃₁-R₃₃, D and E is/are or comprise(s) an aryl.

Exemplary such compounds include Compounds 1, 5, 45 and 75, or Compounds 1, 5 and 45 (see, Table B).

According to some of any of the embodiments described herein for Formula III, at least one or both of R₃₁ and R₃₃ is or comprises an aryl.

According to some of any of the embodiments described herein for Formula III, each of R₃₁ and R₃₃ is or comprises an aryl, which can be the same or different.

According to some of any of the embodiments described herein for Formula III, each of R₃₁ and R₃₃ is a phenyl, which can be the same or different with respect to its substitution.

The aryl, e.g., phenyl, can be unsubstituted or substituted by 1, 2, 3, 4 or 5 substituents, which can be the same or different.

According to some of any of the embodiments described herein for Formula III, at least one of R₃₁ and R₃₃ is an aryl (e.g., phenyl) that is a substituted aryl (e.g., an aryl such as phenyl substituted one or two substituents.

According to some of any of the embodiments described herein for Formula III, each of R₃₁ and R₃₃ is an aryl (e.g., phenyl) that is a substituted aryl (e.g., an aryl such as phenyl substituted one or two substituents.

According to some of any of the embodiments described herein for Formula III, each of R₃₁ and R₃₃ is an aryl (e.g., phenyl) that is substituted by alkyl, hydroxy and/or alkoxy. The alkyl and the alkoxy are preferably of a lower alkyl, e.g., of 1 to 4 carbon atoms in length, as described herein. In exemplary embodiments, the alkyl is methyl. In exemplary embodiments, the alkoxy is methoxy.

In exemplary embodiments, one of R₃₁ and R₃₃ is phenyl substituted by hydroxy and the other is phenyl substituted by alkoxy. In some embodiments, one of these phenyls is further substituted by an alkyl as described herein.

In exemplary embodiments, each of R₃₁ and R₃₃ is phenyl substituted by alkoxy. In some embodiments, one of these phenyls is further substituted by an alkyl as described herein.

According to some of any of the embodiments described herein for Formula III, R₃₂ is halo (e.g., chloro).

According to some of any of the embodiments described herein for Formula III, D and E are each independently selected from hydrogen, alkyl, cycloalkyl, and aryl.

According to some of any of the embodiments described herein for Formula III, at least one or each of D and E is hydrogen.

According to some of any of the embodiments described herein for Formula III, at least one or each of D and E is hydrogen and R₃₂ is halo (e.g., chloro).

According to some of any of the embodiments described herein for Formula III, at least one or each of D and E is hydrogen and each of R₃₁ and R₃₃ is an aryl (e.g., phenyl) that is substituted by alkyl, hydroxy and/or alkoxy, as described herein.

According to some of any of the embodiments described herein for Formula III, at least one or each of D and E is hydrogen, R₃₂ is halo (e.g., chloro) and each of R₃₁ and R₃₃ is an aryl (e.g., phenyl) that is substituted by alkyl, hydroxy and/or alkoxy, as described herein.

Exemplary compounds according to these embodiments are Compounds 5 and 45 (see, Table B).

According to some of any of the embodiments described herein for Formula III, at least one or each of R₃₁ and R₃₃ is an alkyl, preferably a lower alkyl of 1 to 4 carbon atoms as described herein (e.g., methyl).

According to some of any of the embodiments described herein for Formula III, R₃₂ is hydrogen.

According to some of any of the embodiments described herein for Formula III, at least one of D and E is other than hydrogen, and can be, for example, a sulfonyl, a carbonate, a carbonyl, a thiocarbonate, a thiocarbonyl, a sulfonamide, a carbamate, a thiocarbamate, and like moieties, each can be substituted or unsubstituted. According to some of any of these embodiments, the other one of D and E is hydrogen.

According to some of any of the embodiments described herein for Formula III, at least one of D and E is an arylsulfonyl.

According to some of any of the embodiments described herein for Formula III, at least one or each of R₃₁ and R₃₃ is an alkyl, preferably a lower alkyl of 1 to 4 carbon atoms as described herein (e.g., methyl), and at least one of D and E is an arylosulfonyl. According to some of these embodiments, R₃₂ is hydrogen.

According to some of any of the embodiments described herein for Formula III, when one of D and E is an aryl sulfonyl (a sulfonyl as described herein in which R′ is aryl such phenyl, the aryl (e.g. phenyl) is substituted. According to some of these embodiments, the aryl (e.g., phenyl) is substituted by an amide (—NH—C(═O)—R′), which is substituted by an aryl or a heteroaryl (such that R′ is an aryl or heteroaryl). The aryl or heteroaryl can be unsubstituted or substituted. In some embodiments, it is substituted by an amine, as defined herein. The amine can be primary, secondary or tertiary. When the amine is secondary or tertiary, one or both of R′ and R″ can be an alkyl, cycloalkyl or aryl, preferably aryl such as phenyl.

An exemplary compound according to these embodiments is Compound 75 (see, Table B).

According to some of any of the embodiments described herein for Formula III, R₃₁ and R₃₂, or R₃₂ and R₃₃ form together a nitrogen-containing heteroaryl, for example, pyridine, pyrimidine, pyrrole, pyrazine, etc. An exemplary heteroaryl is pyrazine. The heteroaryl can be substituted or unsubstituted. In exemplary embodiments, the heteroaryl is substituted, for example, by at least one (e.g., two) aryl(s) (e.g., phenyl(s)).

According to some of any of the embodiments described herein for Formula III, R₃₁ and R₃₂ from together a pyrazine substituted by one or two phenyls.

According to some of any of the embodiments described herein for Formula III, R₃₃ is an amine, preferably a substituted amine (e.g., a secondary or tertiary amine as defined herein. The secondary or tertiary amine can include as the one or more substituent an alkyl or a cycloalkyl, which can be substituted or unsubstituted. In exemplary embodiments, R₃₃ is an amine substituted by an aminoalkyl. According to some embodiments, the alkyl is a lower alkyl as described herein.

According to some of any of the embodiments described herein for Formula III, at least one of D and E is an alkyl, and in some embodiments the alkyl is a substituted alkyl, for example, an aminoalkyl as described herein.

According to some embodiments, the alkyl is a lower alkyl as described herein.

According to some of any of the embodiments described herein for Formula III, R₃₁ and R₃₂ from together a nitrogen-containing heteroaryl (e.g., a pyrazine substituted by one or two phenyls) and R₃₃ is an amine.

According to some of any of the embodiments described herein for Formula III, R₃₁ and R₃₂ from together a nitrogen-containing heteroaryl (e.g., a pyrazine substituted by one or two phenyls) and R₃₃ is an amine substituted by an alkyl such as an aminoalkyl.

According to some of any of the embodiments described herein for Formula III, R₃₁ and R₃₂ from together a nitrogen-containing heteroaryl (e.g., a pyrazine substituted by one or two phenyls), R₃₃ is an amine, and at least one of D and E is an alkyl, preferably an aminoalkyl as described herein.

According to some of any of the embodiments described herein for Formula III, R₃₁ and R₃₂ from together a nitrogen-containing heteroaryl (e.g., a pyrazine substituted by one or two phenyls), R₃₃ is an amine substituted by an alkyl such as an aminoalkyl, and at least one of D and E is an alkyl, preferably an aminoalkyl as described herein.

An exemplary compound according to these embodiments is Compound 1 (see, Table B).

According to another aspect of some embodiments of the present invention the compounds are collectively represented by Formula IV:

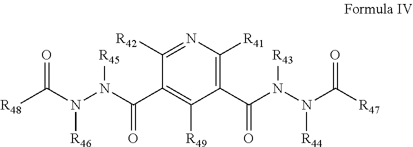

or a pharmaceutically acceptable salt thereof,
wherein:
R₄₃-R₄₆ are each independently hydrogen, alkyl, cycloalkyl and aryl;
R₄₁, R₄₂ and R₄₉ are each independently selected from hydrogen, alkyl, cycloalkyl, aryl, hydroxy, alkoxy, aryloxy, thiol, thioalkoxy, thioaryloxy, amine, imine, halo, nitrile (cyano), nitro, amide, hydrazine, hydrazide, carboxylate, thiocarboxylate, carbamate, thiocarbamate, sulfonyl, sulfinyl, sulfonamide, carbonate, thiocarbonate, sulfoxide, thiosulfate, sulfate, sulfite, thiosulfite, phosphonate, urea, thiourea, guanyl and guanidyl; and
R₄₇ and R₄₈ are each independently selected from alkyl, cycloalkyl, aryl, heteroaryl and heteroalicyclic.

According to some of any of the embodiments described herein for Formula IV, R₄₁ and R₄₂ are each independently an alkyl, preferably a lower alkyl of 1 to 4 carbon atoms in length (e.g., each is methyl).

According to some of any of the embodiments described herein for Formula IV, R₄₃-R₄₆ and R₄₉ are each hydrogen.

According to some of any of the embodiments described herein for Formula IV, R₄₇ and R₄₈ are each independently an alkyl, an alkaryl such as benzyl.

According to some of any of the embodiments described herein for Formula IV, R₄₁ and R₄₂ are each independently an alkyl, preferably a lower alkyl of 1 to 4 carbon atoms in length (e.g., each is methyl), and R₄₇ and R₄₈ are each independently an alkyl, an alkaryl such as benzyl. According to some of these embodiments, R₄₃-R₄₆ and R₄₉ are each hydrogen.

An exemplary compound according to these embodiments is Compound 84 (see, Table B).

According to an aspect of some embodiments of the present invention, the compound is one or more of the compounds presented in Table B.

According to an aspect of some embodiments of the present invention, the compound is one or more of Compounds 1, 5, 18, 29, 42, 45, 47, 69, 71, 73, 75 and 84, as presented in Table B.

According to an aspect of some embodiments of the present invention, the compound is one or more of Compounds 1, 5, 18, 29, 42, 45, 47, 69, 71, 73 and 84, as presented in Table B, for use in treating cancer.

According to an aspect of some embodiments of the present invention, the compound is one or more of Compounds 5, 42, 47, 69, 75 and 84, as presented in Table B, for use in treating cancer.

According to an aspect of some embodiments of the present invention, the compound is one or more of Compounds 5, 42, 47, 69 and 84, as presented in Table B.

According to some of any of the embodiments described herein, a compound as described herein in any of the respective embodiments and any combination therein (a compound of Formula I, II, III, or IV or a compound as presented in Table B), is capable of interfering with an interaction between PD1 and PD-L1, as described herein.

Uses:

According to an aspect of some embodiments of the present invention, a compound as described herein in any of the respective embodiments and any combination thereof (e.g., a compound of Formula I, II, III, or IV or a compound as presented in Table B) is capable of, or is usable in, treating cancer.

According to an aspect of some embodiments of the present invention, there is provided a method of treating a cancer in a subject in need thereof, the method comprising administering to the subject a therapeutically effective amount of a compound as described herein in any of the respective embodiments and any combination thereof (e.g., a compound of Formula I, II, III, or IV or a compound as presented in Table B), thereby treating the cancer.

According to an aspect of some embodiments of the present invention, there is provided a use of a compound as described herein in any of the respective embodiments and any combination thereof (e.g., a compound of Formula I, II, III, or IV or a compound as presented in Table B) in the manufacture of a medicament for treating cancer.

According to an aspect of some embodiments of the present invention, there is provided a use of a compound as described herein in any of the respective embodiments and any combination thereof (e.g., a compound of Formula I, II, III, or IV or a compound as presented in Table B) in treating cancer.

As used herein, the terms “cancer” and “tumor” are interchangeably used. The terms refer to a malignant growth and/or tumor caused by abnormal and uncontrolled cell proliferation (cell division). The term “cancer” encompasses tumor metastases.

The term “cancer cell” describes the cells forming the malignant growth or tumor.

Non-limiting examples of cancers and/or tumor metastases which can be treated according to some embodiments of any of the embodiments described herein relating to cancer (including any of the aspects described herein) include any solid or non-solid cancer and/or tumor metastasis, including, but not limiting to, tumors of the gastrointestinal tract (e.g., colon carcinoma, rectal carcinoma, colorectal carcinoma, colorectal cancer, colorectal adenoma, hereditary nonpolyposis type 1, hereditary nonpolyposis type 2, hereditary nonpolyposis type 3, hereditary nonpolyposis type 6; colorectal cancer, hereditary nonpolyposis type 7, small and/or large bowel carcinoma, esophageal carcinoma, tylosis with esophageal cancer, stomach carcinoma, pancreatic carcinoma, pancreatic endocrine tumors), endometrial carcinoma, dermatofibrosarcoma protuberans, gallbladder carcinoma, biliary tract tumors, prostate cancer, prostate adenocarcinoma, renal cancer (e.g., Wilms' tumor type 2 or type 1), liver cancer (e.g., hepatoblastoma, hepatocellular carcinoma, hepatocellular cancer), bladder cancer, embryonal rhabdomyosarcoma, germ cell tumor, trophoblastic tumor, testicular germ cells tumor, immature teratoma of ovary, uterine, epithelial ovarian, sacrococcygeal tumor, choriocarcinoma, placental site trophoblastic tumor, epithelial adult tumor, ovarian carcinoma, serous ovarian cancer, ovarian sex cord tumors, cervical carcinoma, uterine cervix carcinoma, small-cell and non-small cell lung carcinoma, nasopharyngeal, breast carcinoma (e.g., ductal breast cancer, invasive intraductal breast cancer, sporadic breast cancer, susceptibility to breast cancer, type 4 breast cancer, breast cancer-1, breast cancer-3, breast-ovarian cancer), squamous cell carcinoma (e.g., in head and neck), neurogenic tumor, astrocytoma, ganglioblastoma, neuroblastoma, lymphomas (e.g., Hodgkin's disease, non-Hodgkin's lymphoma, B-cell lymphoma, Diffuse large B-cell lymphoma (DLBCL), Burkitt lymphoma, cutaneous T-cell lymphoma, histiocytic lymphoma, lymphoblastic lymphoma, T-cell lymphoma, thymic lymphoma), gliomas, adenocarcinoma, adrenal tumor, hereditary adrenocortical carcinoma, brain malignancy (tumor), various other carcinomas (e.g., bronchogenic large cell, ductal, Ehrlich-Lettre ascites, epidermoid, large cell, Lewis lung, medullary, mucoepidermoid, oat cell, small cell, spindle cell, spinocellular, transitional cell, undifferentiated, carcinosarcoma, choriocarcinoma, cystadenocarcinoma), ependimoblastoma, epithelioma, erythroleukemia (e.g., Friend, lymphoblast), fibrosarcoma, giant cell tumor, glial tumor, glioblastoma (e.g., multiforme, astrocytoma), glioma hepatoma, heterohybridoma, heteromyeloma, histiocytoma, hybridoma (e.g., B-cell), hypernephroma, insulinoma, islet tumor, keratoma, leiomyoblastoma, leiomyosarcoma, leukemia (e.g., acute lymphatic leukemia, acute lymphoblastic leukemia, acute lymphoblastic pre-B cell leukemia, acute lymphoblastic T cell leukemia, acute megakaryoblastic leukemia, monocytic leukemia, acute myelogenous leukemia, acute myeloid leukemia, acute myeloid leukemia with eosinophilia, B-cell leukemia, basophilic leukemia, chronic myeloid leukemia, chronic B-cell leukemia, eosinophilic leukemia, Friend leukemia, granulocytic or myelocytic leukemia, hairy cell leukemia, lymphocytic leukemia, megakaryoblastic leukemia, monocytic leukemia, monocytic-macrophage leukemia, myeloblastic leukemia, myeloid leukemia, myelomonocytic leukemia, plasma cell leukemia, pre-B cell leukemia, promyelocytic leukemia, subacute leukemia, T-cell leukemia, lymphoid neoplasm, predisposition to myeloid malignancy, acute nonlymphocytic leukemia), lymphosarcoma, melanoma, mammary tumor, mastocytoma, medulloblastoma, mesothelioma, metastatic tumor, monocyte tumor, multiple myeloma, myelodysplastic syndrome, myeloma, nephroblastoma, nervous tissue glial tumor, nervous tissue neuronal tumor, neurinoma, neuroblastoma, oligodendroglioma, osteochondroma, osteomyeloma, osteosarcoma (e.g., Ewing's), papilloma, transitional cell, pheochromocytoma, pituitary tumor (invasive), plasmacytoma, retinoblastoma, rhabdomyosarcoma, sarcoma (e.g., Ewing's, histiocytic cell, Jensen, osteogenic, reticulum cell), schwannoma, subcutaneous tumor, teratocarcinoma (e.g., pluripotent), teratoma, testicular tumor, thymoma and trichoepithelioma, gastric cancer, fibrosarcoma, glioblastoma multiforme, multiple glomus tumors, Li-Fraumeni syndrome, liposarcoma, lynch cancer family syndrome II, male germ cell tumor, mast cell leukemia, medullary thyroid, multiple meningioma, endocrine neoplasia myxosarcoma, paraganglioma, familial nonchromaffin, pilomatricoma, papillary, familial and sporadic, rhabdoid predisposition syndrome, familial, rhabdoid tumors, soft tissue sarcoma, and Turcot syndrome with glioblastoma.

According to some of any of the embodiments of the present invention, the cancer is selected from leukemia, melanoma, lung cancer, lymphoma, myeloma, ovarian cancer, brain cancer, prostate cancer, pancreatic cancer, bladder cancer, colorectal cancer, breast cancer, renal cancer, hepatocellular cancer, hepatoblastoma, cervical cancer, gastric cancer, esophageal cancer, head and neck cancer, neuroendocrine cancer, CNS cancer, bone cancer, soft tissue sarcoma, non-small cell lung cancer, small-cell lung cancer, colon cancer, testicular cancer, adrenocortical carcinoma, dermatofibrosarcoma protuberans, endometrial carcinoma, glioblastoma, glomus tumor, Li-Fraumeni syndrome, liposarcoma, medulloblastoma, meningioma, thyroid cancer, paraganglioma, pilomatricoma, adenocarcinoma, renal cell carcinoma, retinoblastoma, osteosarcoma, myxosarcoma, neuroblastoma, rhabdomyosarcoma, rhabdoid tumors and uterine cervix carcinoma.

According to some of any of the embodiments described herein, a compound as described herein in any of the respective embodiments and any combination thereof is capable of interfering with an interaction between PD-1 and PD-L1.

According to some of any of the embodiments described herein, the cancer is characterized by overexpression of PD-1.

According to some of any of the embodiments described herein, the cancer is lung cancer. Examples of lung cancers which may be treated in the context of some embodiments of the invention include, without limitation, large (non-small) cell lung cancer and small cell lung cancer.

According to some of any of the embodiments described herein, the cancer is melanoma. Examples of melanoma treatable in the context of the present embodiments include, without limitation, superficial spreading melanoma, lentigo melanoma, acral lentigous melanoma and nodular melanoma.

According to some of any of the embodiments described herein, the cancer is breast cancer. Examples of melanoma treatable in the context of the present embodiments include, without limitation, Ductal carcinoma in situ (DCIS), Invasive breast cancer (ILC or IDC), Triple-negative breast cancer, inflammatory breast cancer, Paget disease of the breast, Angiosarcoma and Phyllodes tumor.

According to some of any of the embodiments described herein, the cancer is colorectal cancer. Examples of colorectal cancer treatable in the context of the present embodiments include, without limitation, Adenocarcinoma, Gastrointestinal carcinoid tumors, Primary colorectal lymphomas, Gastrointestinal stromal tumors, Leiomyosarcomas, Squamous cell carcinomas, Familial adenomatous polyposis (FAP), Turcot Syndrome, Peutz-Jeghers Syndrome (PJS), Familial Colorectal Cancer (FCC), and Juvenile Polyposis Coli.

According to some of any of the embodiments described herein, the cancer is bladder cancer. Examples of bladder cancers treatable in the context of the present embodiments include, without limitation, transitional cell (urothelial) carcinoma (TCC), including papillary carcinoma and flat carcinomas, non-invasive bladder cancer, invasive bladder cancer, recurrent bladder cancer, metastatic bladder cancer, Squamous cell carcinoma, adenocarcinoma of the bladder, small-cell carcinoma and sarcoma.

Subjects to be treated according to the present embodiments includes subjects suffering from cancer, including non-treated subjects, subjects that have received one more anti-cancer therapy, and subjected with recurrent and/or metastasizing tumors.

In some embodiments of any one of the embodiments described herein relating to treatment of cancer, the cancer treatment further comprises administering at least one additional anti-cancer agent (i.e., in addition to the compound described hereinabove) and/or anti-cancer therapy, including radiotherapy, chemotherapy, phototherapy and photodynamic therapy, surgery, nutritional therapy, ablative therapy, combined radiotherapy and chemotherapy, brachiotherapy, proton beam therapy, targeted therapy (e.g., BRAFi, MEKi, EGFRi, etc.), immunotherapy, cellular therapy and photon beam radiosurgical therapy. Analgesic agents and other treatment regimens are also contemplated.

The additional anti-cancer agent may be any agent used in the medical arts to treat a cancer. Examples of anti-cancer agents include, without limitation, acivicin; aclarubicin; acodazole hydrochloride; acronine; adriamycin; Adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cisplatin; cladribine; combrestatin A-4 phosphate; crisnatol mesylate; cyclophosphamide; cytarabine; dacarbazine; dactinomycin; daunorubicin hydrochloride; decitabine; dexormaplatin; dezaguanine; dezaguanine mesylate; diaziquone; docetaxel; doxorubicin; doxorubicin hydrochloride; droloxifene; droloxifene citrate; dromostanolone propionate; duazomycin; edatrexate; eflornithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; flurocitabine; fosquidone; fostriecin sodium; gemcitabine; gemcitabine hydrochloride; hydroxyurea; idarubicin hydrochloride; ifosfamide; ilmofosine; interferon alfa-2a; interferon alfa-2b; interferon alfa-n1; interferon alfa-n3; interferon beta-Ia; interferon gamma-Ib; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazole; nogalamycin; ombrabulin; ormaplatin; oxisuran; paclitaxel; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofuirin; tirapazamine; topotecan hydrochloride; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine; vincristine sulfate; vindesine; vindesine sulfate; vinepidinee; vinglycinate; vinleurosine; vinorelbine tartrate; vinrosidine; vinzolidine; vorozole; zeniplatin; zinostatin; and zorubicin hydrochloride. Additional anti-cancer agents include those disclosed in Chapter 52, Antineoplastic Agents (Paul Calabresi and Bruce A. Chabner), and the introduction thereto, 1202-1263, of Goodman and Gilman's “The Pharmacological Basis of Therapeutics”, Eighth Edition, 1990, McGraw-Hill, Inc. (Health Professions Division), the contents of which are incorporated herein by reference.

In any of the methods and uses described herein, the compounds as described herein can be used in combination with an additional active agent or therapy that is usable for treating the cancer and/or in modulating an immune response in a subject in need thereof.

According to a specific embodiment, the treatment of cancer is effected in combination with an anti-cancer immune modulator agent.

As used herein, the term “anti-cancer immune modulator agent” refers to an agent capable of eliciting an immune response (e.g. T cell, NK cell) against a cancerous cell.

According to specific embodiment, the agent is selected from the group consisting of a cancer antigen, a cancer vaccine, an anti-cancer antibody, a cytokine capable of inducing activation and/or proliferation of a T cell (e.g. TGF-β) and an immune-check point regulator.

Alternatively or additionally, such modulators may be immune stimulators such as immune-check point regulators which are of specific value in the treatment of cancer.

As used herein the term “immune-check point regulator” refers to a molecule that modulates the activity of one or more immune-check point proteins in an agonistic or antagonistic manner resulting in activation of an immune cell.

As used herein the term “immune-check point protein” refers to a protein that regulates an immune cell activation or function. Immune check-point proteins can be either co-stimulatory proteins (i.e. transmitting a stimulatory signal resulting in activation of an immune cell) or inhibitory proteins (i.e. transmitting an inhibitory signal resulting in suppressing activity of an immune cell). According to specific embodiment, the immune check point protein regulates activation or function of a T cell. Numerous checkpoint proteins are known in the art and include, but not limited to, PD1, PDL-1, B7H2, B7H4, CTLA-4, CD80, CD86, LAG-3, TIM-3, KIR, IDO, CD19, OX40, 4-1BB (CD137), CD27, CD70, CD40, GITR, CD28 and ICOS (CD278).

According to specific embodiments, the immune-check-point regulator is selected form the group consisting of anti-CTLA4, anti-PD-1, and CD40 agonist.

According to specific embodiments, the immune-check point regulator is selected form the group consisting of anti-CTLA4, anti-PD-1, anti-PDL-1, CD40 agonist, 4-1BB agonist, GITR agonist and OX40 agonist.

CTLA4 is a member of the immunoglobulin superfamily, which is expressed on the surface of Helper T cells and transmits an inhibitory signal to T cells upon ligand binding. As used herein, the term “anti-CTLA4” refers to an antagonistic molecule that binds CTLA4 (CD152) and suppresses its suppressive activity. Thus, an anti-CTLA4 prevents the transmission of the inhibitory signal and thereby acts as a co-stimulatory molecule. According to a specific embodiment, the anti-CTLA4 molecule is an antibody.

According to a specific embodiment, the anti-PD1 molecule is an antibody. Numerous anti-PD-1 antibodies are known in the art see e.g. Topalian, et al. NEJM 2012.

As used herein, the tem “anti-PDL-1” refers to an antagonistic molecule that inhibits PD-1 signaling by binding to or inhibiting PD-L1 from binding and/or activating PD-1. Thus, an anti-PD-1 prevents the transmission of the inhibitory signal and thereby acts as a co-stimulatory molecule. According to specific embodiments, the anti-PD-L1 is an anti-PD-L1 antibody. Numerous anti-PDL-1 antibodies are known in the art see e.g. Brahmer, et al. NEJM 2012.

CD40 (CD154) is a co-stimulatory receptor found on antigen presenting cells and transmits an activation signal upon ligand binding. As used herein, the term “CD40 agonist” refers to an agonistic molecule that binds CD40 (CD154) and thereby induces activation of the antigen presenting cell.

OX40 belongs to the TNF receptor super family and leads to expansion of CD4+ and CD8+ T cells. As used herein, the term “OX40 agonist” refers to an agonistic molecule that binds and activates OX40.

GITR (glucocorticoid-induced tumor necrosis factor receptor) is a surface receptor molecule that has been shown to be involved in inhibiting the suppressive activity of T-regulatory cells and extending the survival of T-effector cells. As used herein, the term “GITR agonist” refers to an agonistic molecule that binds and activates GITR. According to a specific embodiment, the GITR agonist is an antibody.

According to an aspect of some embodiments of the present invention there is provided a compound for use in the treatment of cancer or a condition in a patient that is amenable to treatment by inhibiting PD-1, PD-L1 and/or the PD-1/PD-L1 interaction.

According to an aspect of some embodiments of the present invention there is provided a compound for use in modulating an immune response to aid in the treatment of a disease or condition which is characterized by overexpression of PD-1.

According to some of any of the embodiments of the present invention, treating the cancer further comprises administering to the subject one or more immune checkpoint inhibitors or an additional agent known to treat or prevent cancer.

In some embodiments, the “additional immune checkpoint inhibitors” may be an anti-PD-1, an anti-PD-L1 antibody, and/or an anti PD-1/PD-L1 interaction inhibitor. In some embodiments, the anti-PD-L1 antibody may be B7-H1 antibody, BMS 936559 antibody, MPDL3280A (atezolizumab) antibody, MEDI-4736 antibody, MSB0010718C antibody or combinations thereof. According to another embodiment, the anti-PD-1 antibody may be nivolumab antibody, pembrolizumab antibody, pidilizumab antibody or combinations thereof.

According to an aspect of some embodiments of the present invention, there is provided a compound as described herein in any of the respective embodiments and any combination thereof (a compound represented by Formula I, II, III or IV, or a compound selected from the compounds presented in Table B), for use in interfering with an interaction between PD-1 and PD-L1 and/or for treating a medical condition in which interfering with an interaction between PD-1 and PD-L1 is beneficial, and/or in which modulating (e.g., inhibiting) PD-1 and/or PD-L1 function on cells, for example tumor and myeloid cells such as antigen presenting cells, is beneficial.

According to an aspect of some embodiments of the present invention, there is provided a method of interfering with an interaction between PD-1 and PD-L1 in a subject in need thereof (e.g., a subject afflicted by a condition characterized by overexpression of PD-1), which comprises administering to the subject a therapeutically effective amount of a compound as described herein in any of the respective embodiments and any combination thereof (a compound represented by Formula I, II, III or IV, or a compound selected from the compounds presented in Table B).

According to an aspect of some embodiments of the present invention, there is provided a method of interfering with an interaction between PD-1 and PD-L1 in a subject in need thereof (e.g., a subject afflicted by a condition characterized by overexpression of PD-1), and/or of treating a medical condition in which interfering with an interaction between PD-1 and PD-L1 is beneficial, and/or in which modulating (e.g., inhibiting) PD-1 and/or PD-L1 function on cells, for example tumor and myeloid cells such as antigen presenting cells, is beneficial, which comprises administering to the subject a therapeutically effective amount of a compound as described herein in any of the respective embodiments and any combination thereof (a compound represented by Formula I, II, III or IV, or a compound selected from the compounds presented in Table B).

According an aspect of some embodiments of the present invention, there is provided a compound as described herein in any of the respective embodiments and any combination thereof (a compound represented by Formula I, II, III or IV, or a compound selected from the compounds presented in Table B), for use in increasing T-cell function, or in modulating an immune response, or in treating a medical condition associated in which increasing T-cell function is beneficial, in a subject in need thereof.

According an aspect of some embodiments of the present invention, there is provided a method of increasing T-cell function, or in modulating an immune response, or in treating a medical condition associated in which increasing T-cell function is beneficial, in a subject in need thereof, which comprises administering to the subject a therapeutically effective amount of a compound as described herein in any of the respective embodiments and any combination thereof (a compound represented by Formula I, II, III or IV, or a compound selected from the compounds presented in Table B).

As used herein, the term “T cell” refers to a differentiated lymphocyte with a CD3⁺, T cell receptor (TCR)⁺ having either CD4⁺ or CD8⁺ phenotype. The T cell may be either an effector or a regulatory T cells.

As used herein, the term “effector T cells” refers to a T cell that activates or directs other immune cells e.g. by producing cytokines or has a cytotoxic activity e.g., CD4+, Th1/Th2, CD8+ cytotoxic T lymphocyte.

As used herein, the term “regulatory T cell” or “Treg” refers to a T cell that negatively regulates the activation of other T cells, including effector T cells, as well as innate immune system cells. Treg cells are characterized by sustained suppression of effector T cell responses. According to a specific embodiment, the Treg is a CD4+CD25+Foxp3+ T cell.

According to additional aspects of the present embodiments, there is provided a use of the compounds as described herein in any of the respective embodiments and any combination thereof (a compound represented by Formula I, II, III or IV, or a compound selected from the compounds presented in Table B) as a medicament, or in the manufacture of a medicament for treating cancer, or a medicament for interfering with an interaction between PD-1 and PD-L1, or a medicament for increasing T-cell function, or a medicament for modulating an immune response, in a subject in need thereof, as these uses are described herein in any of the respective embodiments.

The compounds according to the present embodiments (a compound represented by Formula I, II, III or IV, or a compound selected from the compounds presented in Table B) are usable in treating any medical condition that is associated with the function of PD1, PDL1, T-cells and related immunosupressor cytokines such as TGF-β on cells, for example, tumor and myeloid cells such as antigen presenting cells.

Exemplary medical conditions treatable by the compounds according to the present embodiments, in addition to cancer, include neurodegenerative diseases and disorders and infectious diseases and disorders.

According to an aspect of some embodiments of the present invention, there is provided a compound as described herein in any of the respective embodiments and any combination thereof (a compound represented by Formula I, II, III or IV, or a compound selected from the compounds presented in Table B) for use in treating a neurodegenerative disease or disorder in a subject in need thereof.

According to an aspect of some embodiments of the present invention, there is provided a use of compound as described herein in any of the respective embodiments and any combination thereof (a compound represented by Formula I, II, III or IV, or a compound selected from the compounds presented in Table B) in the manufacture of a medicament for use in treating a neurodegenerative disease or disorder in a subject in need thereof.

According to an aspect of some embodiments of the present invention, there is provided a method of treating a neurodegenerative disease or disorder in a subject in need thereof, the method comprising administering to the subject a therapeutically effective amount of a compound as described herein in any of the respective embodiments and any combination thereof (a compound represented by Formula I, II, III or IV, or a compound selected from the compounds presented in Table B).

A neurodegenerative disorder can result from an event that causes neuronal cell death. Such an event can be, for example, cerebral ischemia, stroke, traumatic brain injury or bacterial infection.

A neurodegenerative disease includes, for example, chronic neurodegenerative diseases such as, but not limited to, Alzheimer's disease, Huntington's disease, Parkinson's disease, AIDS associated dementia, amyotrophic lateral sclerosis (AML) and multiple sclerosis.

Medical conditions include neurodegenerative diseases, neurological diseases, neurological autoimmune diseases, multiple sclerosis, Alzheimer's disease, myasthenia gravis, motor neuropathies, Guillain-Barre syndrome, neuropathies and autoimmune neuropathies, myasthenic diseases, Lambert-Eaton myasthenic syndrome, paraneoplastic neurological diseases, cerebellar atrophy, paraneoplastic cerebellar atrophy, non-paraneoplastic stiff man syndrome, cerebellar atrophies, progressive cerebellar atrophies, encephalitis, Rasmussen's encephalitis, amyotrophic lateral sclerosis, Sydeham chorea, Gilles de la Tourette syndrome, neuropathies, dysimmune neuropathies; neuromyotonia, acquired neuromyotonia, arthrogryposis multiplex congenital.

According to an aspect of some embodiments of the present invention, there is provided a compound as described herein in any of the respective embodiments and any combination thereof (a compound represented by Formula I, II, III or IV, or a compound selected from the compounds presented in Table B) for use in treating an infectious diseases or disorder in a subject in need thereof.

According to an aspect of some embodiments of the present invention, there is provided a use of compound as described herein in any of the respective embodiments and any combination thereof (a compound represented by Formula I, II, III or IV, or a compound selected from the compounds presented in Table B) in the manufacture of a medicament for use in treating an infectious disease in a subject in need thereof.

According to an aspect of some embodiments of the present invention, there is provided a method of treating an infectious disease in a subject in need thereof, the method comprising administering to the subject a therapeutically effective amount of a compound as described herein in any of the respective embodiments and any combination thereof (a compound represented by Formula I, II, III or IV, or a compound selected from the compounds presented in Table B).

Non-limiting examples of infectious diseases or disorders include chronic infectious diseases or disorders, a subacute infectious disease or disorder, an acute infectious disease or disorder, a viral disease or disorder, a bacterial disease or disorder, a protozoan disease or disorder, a parasitic disease or disorder, a fungal disease or disorder, a mycoplasma disease or disorder, gangrene, sepsis, a prion disease or disorder, influenza, tuberculosis, malaria, acquired immunodeficiency syndrome, and severe acute respiratory syndrome. Exemplary infectious diseases include HIV, encephalitis, meningitis, encephalomyelitis, viral gastroenteritis, viral hepatitis, SARS-CoV, Middle East respiratory syndrome Coronavirus (MERS-CoV), and the recently identified SAR-CoV-2.

In any of the methods and uses described herein, the compounds as described herein can be used in combination with any of the additional agents as described herein.

According to some embodiments, the additional agent comprises one or more cytokine(s) capable of inducing activation and/or proliferation of a T cell, as described herein. Such cytokines include, but are not limited to, IFNα, IFNγ, IL-1, IL-2, IL-6, IL-7, IL-10, IL-12, IL-15, IL-21 and TNFα.

Specific non-limiting examples of cytokines and cytokines agonists that can be used according to some embodiments of the invention include:

IL-2 (produced by Roche); IL21 (produced by BMY); ALT-803 (IL15 superagonist combined with a soluble IL-15a receptor, produced by Altor Bioscience); Darleukin (L19-IL2, human IL-2 conjugated with an antibody (L19) that is specific to the EDB region of fibronectin, produced by Philogen); Denenicokin [BMS-982470, a recombinant human peptide homologous to IL-21, produced by Bristol-Myers Squibb (ZymoGenetics)]; and Immunopulse (delivery of DNA-based IL-12 leading to localized expression of IL-12 in the tumor microenvironment, produced by Oncosec Medical.

Other agents that can be used in combination with a compound as described herein, in any of the methods and uses described herein include, for example, CTLA4, LAG-3, TIM-3, KIRs (killer cell Ig-like receptors), IDO (indoleamine 2,3-dioxygenase), OX40, gene symbol TNFRSF4, also known as CD134, Tumor necrosis factor receptor superfamily, member 4, TNFRSF4, TXGP1L, ACT35 and IMD16, CD137, gene symbol TNFRSF9, also known as 4-1BB and Tumor Necrosis Factor Receptor Superfamily, Member 9 and TNFRSF9, CD27, gene symbol CD27, also known as Tumor Necrosis Factor Receptor Superfamily, Member 7, TNFRSF7 and S152, CD40, gene symbol CD40, also known as Tumor Necrosis Factor Receptor Superfamily, Member 5 and TNFRSF5, GITR (glucocorticoid-induced tumor necrosis factor receptor), gene symbol TNFRSF18 is also known as TNF receptor superfamily 18, TNFRSF18, AITR and CD357, and CD28, gene symbol CD28, also known as Tp44, ICOS (Inducible T-cell co-stimulator), gene symbol ICOS, also known as CD278, AILIM and CVID1; and agonists and/or activators thereof.

Pharmaceutical Compositions:

In any of the methods and uses described herein, the compounds as described herein can be used either per se or as a part of a pharmaceutical composition, which optionally further comprises a pharmaceutically acceptable carrier.

The compounds described herein according to any of the aspects of embodiments of the invention described herein can be utilized (e.g., administered to a subject) per se or in a pharmaceutical composition where the compound is mixed with suitable carriers or excipients.

As used herein a “pharmaceutical composition” refers to a preparation of one or a compound according to any of the embodiments described herein with other chemical components such as physiologically suitable carriers and excipients. The purpose of a pharmaceutical composition is to facilitate administration of a compound to an organism.

Hereinafter, the phrases “physiologically acceptable carrier” and “pharmaceutically acceptable carrier” which may be interchangeably used refer to a carrier or a diluent that does not cause significant irritation to an organism and does not abrogate the biological activity and properties of the administered compound. An adjuvant is included under these phrases.

Herein the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols.

When utilized per se or in a pharmaceutically acceptable composition, the compound per se (that is, not including, weight of carriers or excipients co-formulated with the compound, as described herein) is optionally at least 80% pure (by dry weight), optionally at least 90% pure (by dry weight), at least 95% pure (by dry weight), at least 98% pure (by dry weight), and optionally at least 99% pure (by dry weight). Purity may be enhanced, e.g., by removing impurities associated with synthesis of the compound or isolation of the compound from a natural source, by any suitable technique known in the art. Techniques for formulation and administration of drugs may be found in “Remington's Pharmaceutical Sciences,” Mack Publishing Co., Easton, PA, latest edition, which is incorporated herein by reference.

Suitable routes of administration may, for example, include oral, rectal, transmucosal, especially transnasal, intestinal or parenteral delivery, including intramuscular, subcutaneous and intramedullary injections as well as intrathecal, direct intraventricular, intracardiac, e.g., into the right or left ventricular cavity, into the common coronary artery, intravenous, intraperitoneal, intranasal, or intraocular injections.

Alternately, one may administer the pharmaceutical composition in a local rather than systemic manner, for example, via injection of the pharmaceutical composition directly into a tissue region of a patient.

The term “tissue” refers to part of an organism consisting of cells designed to perform a function or functions. Examples include, but are not limited to, brain tissue, retina, skin tissue, hepatic tissue, pancreatic tissue, breast tissue, bone, cartilage, connective tissue, blood tissue, muscle tissue, cardiac tissue brain tissue, vascular tissue, renal tissue, pulmonary tissue, gonadal tissue, hematopoietic tissue.

Pharmaceutical compositions of some embodiments of the invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with some embodiments of the invention thus may be formulated in conventional manner using one or more physiologically acceptable carriers comprising excipients and auxiliaries, which facilitate processing of the active ingredients into preparations which, can be used pharmaceutically. Proper formulation is dependent upon the route of administration chosen.

For injection, the active ingredients of the pharmaceutical composition may be formulated in aqueous solutions, preferably in physiologically compatible buffers such as Hank's solution, Ringer's solution, or physiological salt buffer. For transmucosal administration, penetrants appropriate to the barrier to be permeated are used in the formulation. Such penetrants are generally known in the art.

For oral administration, the pharmaceutical composition can be formulated readily by combining the active compounds with pharmaceutically acceptable carriers well known in the art. Such carriers enable the pharmaceutical composition to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose; and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active compound doses.

Pharmaceutical compositions which can be used orally include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

For administration by nasal inhalation, the active ingredients for use according to some embodiments of the invention are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in a dispenser may be formulated containing a powder mix of the active compound and a suitable powder base such as lactose or starch.

The pharmaceutical composition described herein may be formulated for parenteral administration, e.g., by bolus injection or continuous infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the active preparation in water-soluble form. Additionally, suspensions of the active ingredients may be prepared as appropriate oily or water based injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents which increase the solubility of the active ingredients to allow for the preparation of highly concentrated solutions.

Alternatively, the active ingredient may be in powder form for constitution with a suitable vehicle, e.g., sterile, pyrogen-free water based solution, before use.

The pharmaceutical composition of some embodiments of the invention may also be formulated in rectal compositions such as suppositories or retention enemas, using, e.g., conventional suppository bases such as cocoa butter or other glycerides.

Pharmaceutical compositions suitable for use in context of some embodiments of the invention include compositions wherein the active ingredients are contained in an amount effective to achieve the intended purpose. More specifically, a therapeutically effective amount means an amount of the active ingredient(s) effective to prevent, alleviate or ameliorate symptoms of a disorder (e.g., cancer or metastatic cancer) or prolong the survival of the subject being treated.

Determination of a therapeutically effective amount is well within the capability of those skilled in the art, especially in light of the detailed disclosure provided herein.

For any preparation used in the methods of the invention, the therapeutically effective amount or dose can be estimated initially from in vitro and cell culture assays. For example, a dose can be formulated in animal models to achieve a desired concentration or titer. Such information can be used to more accurately determine useful doses in humans.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition (see, e.g., Fingl et al. (1975), in “The Pharmacological Basis of Therapeutics”, Ch. 1 p. 1).

Dosage amount and interval may be adjusted individually to provide inhibitory levels of the active ingredient are sufficient to induce or suppress the biological effect (minimal effective concentration, MEC). The MEC will vary for each preparation, but can be estimated from in vitro data, e.g., as described herein. Dosages necessary to achieve the MEC will depend on individual characteristics and route of administration. Detection assays can be used to determine plasma concentrations.

In some embodiments of any of the embodiments described herein, an effective amount of the compound is less than 100 μM. In some embodiments, an effective amount is less than 10 μM. In some embodiments, an effective amount is less than 5 μM. In some embodiments, an effective amount is less than 1 μM. In some embodiments, an effective amount is less than 0.5 μM. In some embodiments, an effective amount is less than 0.1 μM.

Depending on the severity and responsiveness of the condition to be treated, dosing can be of a single or a plurality of administrations, with course of treatment lasting from several days to several weeks or until cure is effected or diminution of the disease state is achieved.

The amount of a composition to be administered will, of course, be dependent on the subject being treated, the severity of the affliction, the manner of administration, the judgment of the prescribing physician, etc.

Compositions of some embodiments of the invention may, if desired, be presented in a pack or dispenser device, such as an FDA approved kit, which may contain one or more unit dosage forms containing the active ingredient. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert. Compositions comprising a preparation of the invention formulated in a compatible pharmaceutical carrier may also be prepared, placed in an appropriate container, and labeled for treatment of an indicated condition, as described herein.

It will be appreciated that the compounds described herein can be provided alone or in combination with other active ingredients, which are well known in the art for alleviating the medical condition.

Thus, for example, the compound may be administered with an immunomodulator, either together in a co-formulation or in separate formulations.

According to a specific embodiment, the treatment of cancer (and other hyperproliferative disorders) is effected in combination with an anti-cancer immune modulator agent.

As used herein, the term “anti-cancer immune modulator agent” refers to an agent capable of eliciting an immune response (e.g. T cell, NK cell) against a cancerous cell.

According to specific embodiment, the agent is selected from the group consisting of a cancer antigen, a cancer vaccine, an anti-cancer antibody, a cytokine capable of inducing activation and/or proliferation of a T cell (e.g. TGF-β) and an immune-check point regulator.

Alternatively or additionally, such modulators may be immune stimulators such as immune-check point regulators which are of specific value in the treatment of cancer.

The compound may be administered with an additional anti-cancer agent or therapy, as described herein in any of the respective embodiments, either together in a co-formulation (e.g., in the same pharmaceutical composition) or in separate formulations.

A pharmaceutical composition as described herein can further comprise any of the additional agents as described herein, or alternatively, be identified for use in combination with an additional agent as described herein.

According to another aspect described herein, there is provided a kit for the treatment of a condition (e.g., treatment of cancer) as described herein, the kit comprising a packaging material packaging the compound described herein.

In some of these embodiments, the kit further comprises an additional agent as described herein in any of the respective embodiments, and the two agents are packaged individually within the kit.

In some of these embodiments, the kit further comprises instructions to use the compound in combination with an additional agent (e.g., an additional anti-cancer agent or therapy) as described herein in any of the respective embodiments.

According to an aspect of some embodiments of the present invention there is provided a pharmaceutical composition comprising a small molecule compound as described herein in any of the respective embodiments, optionally in combination with a pharmaceutically acceptable carrier, and further optionally in combination with additional active agent (e.g., anti-cancer agent or therapy) as described herein in the respective embodiments.

According to an aspect of some embodiments of the present invention there is provided a small molecule compound as described herein in any of the respective embodiments for use as a medicament or in the manufacture of a medicament.

The medicament can be a pharmaceutical composition as described herein in any of the respective embodiments.

The medicament can be for use in treating any of the medical conditions, diseases and/or disorders as described herein.

Screening:

According to an aspect of some embodiments of the present invention there is provided a method of identifying a compound (a small molecule, non-peptidic, non-proteinacious compound) capable of interfering with an interaction between PD-1 and PD-L1, essentially as described herein.

According to an aspect of some embodiments of the present invention there is provided a method of identifying a lead candidate small molecule compound for treating cancer. According to these embodiments, the method combines computational (in silico) screening of a database of small molecule compounds, for identifying small molecule compounds that are capable of interacting with a binding pocket of PD-L1, and an in vitro assay for determining which of the identified compounds is indeed capable of inhibiting of an interaction between FD-1 and PD-L1. Compounds identified as capable of inhibiting this interaction are determined as lead candidates for treating cancer.

According to some of any of the embodiments of this aspect of the present invention, the computational screening is based on an interaction of the compounds in the screened database with a three-dimensional (3D) PD-L1 structure (PDBID: 5J89) retrieved from Protein Data Bank (www(dot)rcsb(dot)org), as depicted in FIG. 2A.

According to some of any of the embodiments of this aspect of the present invention, the computational screening is performed as described herein in the Examples section that follows. Once computational screening is completed, further computational optimization steps can be performed, such as described herein in the Examples section that follows, and is schematically depicted in FIG. 2B.

According to some of any of the embodiments of this aspect of the present invention, the in vitro assay is performed as described herein in the Examples section that follows.

The hits retrieved in the in silico screening (small molecule compounds identified as capable of interacting with a binding pocket of PD-L1) are subjected to an assay that determines inhibition of an interaction between PD-1 and PD-L1, to thereby identify compounds capable of inhibiting said interaction.

Compounds identified as capable of inhibiting of an interaction between PD-1 and PD-L1 in the in vitro assay are determined as lead candidates for treating cancer.

Optionally, the identified lead candidates are further characterized in activity and drugability assays in order to select compounds that are most suited for the intended therapeutic use, using methods well known in the art.

In some embodiments, lead compounds are assessed for their anti-cancer activity and/or effect on immune response using cancer cells obtained from a tumor to be treated (e.g., cancer cells dissected from a subject to be treated).

As used herein the term “about” refers to ±10% or ±5%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

Herein throughout, the phrase “linking moiety” or “linking group” describes a group that connects two or more moieties or groups in a compound. A linking moiety is typically derived from a bi- or tri-functional compound, and can be regarded as a bi- or tri-radical moiety, which is connected to two or three other moieties, via two or three atoms thereof, respectively.

Exemplary linking moieties include a hydrocarbon moiety or chain, optionally interrupted by one or more heteroatoms, as defined herein, and/or any of the chemical groups listed below, when defined as linking groups.

When a chemical group is referred to herein as “end group” it is to be interpreted as a substituent, which is connected to another group via one atom thereof.

Herein throughout, the term “hydrocarbon” collectively describes a chemical group composed mainly of carbon and hydrogen atoms. A hydrocarbon can be comprised of alkyl, alkene, alkyne, aryl, and/or cycloalkyl, each can be substituted or unsubstituted, and can be interrupted by one or more heteroatoms. The number of carbon atoms can range from 2 to 20, and is preferably lower, e.g., from 1 to 10, or from 1 to 6, or from 1 to 4. A hydrocarbon can be a linking group or an end group.

As used herein, the term “amine” describes both a —NR′R″ group and a —NR′— group, wherein R′ and R″ are each independently hydrogen, alkyl, cycloalkyl, aryl, as these terms are defined hereinbelow.

The amine group can therefore be a primary amine, where both R′ and R″ are hydrogen, a secondary amine, where R′ is hydrogen and R″ is alkyl, cycloalkyl or aryl, or a tertiary amine, where each of R′ and R″ is independently alkyl, cycloalkyl or aryl.

Alternatively, R′ and R″ can each independently be hydroxyalkyl, trihaloalkyl, cycloalkyl, alkenyl, alkynyl, aryl, heteroaryl, heteroalicyclic, amine, halide, sulfonate, sulfoxide, phosphonate, hydroxy, alkoxy, aryloxy, thiohydroxy, thioalkoxy, thioaryloxy, cyano, nitro, azo, sulfonamide, carbonyl, C-carboxylate, O-carboxylate, N-thiocarbamate, O-thiocarbamate, urea, thiourea, N-carbamate, O-carbamate, C-amide, N-amide, guanyl, guanidine and hydrazine.

Further alternatively, R′ and R″ form together a heteroalicyclic nitrogen-containing ring.

Herein throughout, an “amine-containing group” describes a chemical group that comprises or consists of at least one —NR′— or —NR′R″ group, with R′ and R″ is each independently hydrogen, alkyl, or cycloalkyl, or R′ and R″ form together a heterocyclic (e.g., alicyclic) group, or as defined hereinafter.

An amine-containing group can alternatively be a chemical group that comprises one or more —NR′— or —NR′R″ group(s) as defined herein, as part of a larger group that comprises additional chemical groups. Examples of such groups include, without limitation, amide, thioamide, carbamate, thiocarbamate, or polyamine-containing groups such as, but not limited to, guanyl, guanidyl, hydrazine, hydrazide, thiohydrazide, urea, and thiourea.

Preferably, one or more amine groups in an amine or polyamine-containing group is such that has pKa around physiological pH (6-8, or about 7), such that it is not fully protonated in physiological environment, as in the case of, for example, guanidine.

The term “alkyl” describes a saturated aliphatic hydrocarbon including straight chain and branched chain groups. Preferably, the alkyl group has 1 to 30, or 1 to 20 carbon atoms. Whenever a numerical range; e.g., “1-20”, is stated herein, it implies that the group, in this case the alkyl group, may contain 1 carbon atom, 2 carbon atoms, 3 carbon atoms, etc., up to and including 20 carbon atoms. The alkyl group may be substituted or unsubstituted.

The alkyl group can be an end group, as this phrase is defined hereinabove, wherein it is attached to a single adjacent atom, or a linking group, as this phrase is defined hereinabove, which connects two or more moieties via at least two carbons in its chain. When the alkyl is a linking group, it is also referred to herein as “alkylene” or “alkylene chain”.

Alkene and Alkyne, as used herein, are an alkyl, as defined herein, which contains one or more double bond or triple bond, respectively.

The term “cycloalkyl” describes an all-carbon monocyclic ring or fused rings (i.e., rings which share an adjacent pair of carbon atoms) group where one or more of the rings does not have a completely conjugated pi-electron system. Examples include, without limitation, cyclohexane, adamantine, norbornyl, isobornyl, and the like. The cycloalkyl group may be substituted or unsubstituted.

The cycloalkyl group can be an end group, as this phrase is defined hereinabove, wherein it is attached to a single adjacent atom, or a linking group, as this phrase is defined hereinabove, connecting two or more moieties at two or more positions thereof.

The term “heteroalicyclic” describes a monocyclic or fused ring group having in the ring(s) one or more atoms such as nitrogen, oxygen and sulfur. The rings may also have one or more double bonds. However, the rings do not have a completely conjugated pi-electron system. Representative examples are piperidine, piperazine, tetrahydrofurane, tetrahydropyrane, morpholino, oxalidine, and the like.

The heteroalicyclic may be substituted or unsubstituted. The heteroalicyclic group can be an end group, as this phrase is defined hereinabove, where it is attached to a single adjacent atom, or a linking group, as this phrase is defined hereinabove, connecting two or more moieties at two or more positions thereof.

The term “aryl” describes an all-carbon monocyclic or fused-ring polycyclic (i.e., rings which share adjacent pairs of carbon atoms) groups having a completely conjugated pi-electron system. The aryl group may be substituted or unsubstituted. The aryl group can be an end group, as this term is defined hereinabove, wherein it is attached to a single adjacent atom, or a linking group, as this term is defined hereinabove, connecting two or more moieties at two or more positions thereof.

The term “heteroaryl” describes a monocyclic or fused ring (i.e., rings which share an adjacent pair of atoms) group having in the ring(s) one or more atoms, such as, for example, nitrogen, oxygen and sulfur and, in addition, having a completely conjugated pi-electron system. Examples, without limitation, of heteroaryl groups include pyrrole, furan, thiophene, imidazole, oxazole, thiazole, pyrazole, pyridine, pyrimidine, quinoline, isoquinoline and purine. The heteroaryl group can be an end group, as this phrase is defined hereinabove, where it is attached to a single adjacent atom, or a linking group, as this phrase is defined hereinabove, connecting two or more moieties at two or more positions thereof. Representative examples are pyridine, pyrrole, oxazole, indole, purine and the like.

Whenever an alkyl, cycloalkyl, aryl, alkaryl, heteroaryl, heteroalicyclic, acyl and any other moiety as described herein is substituted, it includes one or more substituents, each can independently be, but are not limited to, hydroxy, alkoxy, thiohydroxy, thioalkoxy, aryloxy, thioaryloxy, alkaryl, alkenyl, alkynyl, sulfonate, sulfoxide, thiosulfate, sulfate, sulfite, thiosulfite, phosphonate, cyano, nitro, azo, sulfonamide, carbonyl, thiocarbonyl, C-carboxylate, O-carboxylate, N-thiocarbamate, O-thiocarbamate, oxo, thiooxo, oxime, acyl, acyl halide, azo, azide, urea, thiourea, N-carbamate, O-carbamate, C-amide, N-amide, guanyl, guanidyl, hydrazine and hydrazide, as these terms are defined herein, unless otherwise indicated.

The terms “halide” or “halo” or “halogen” are used interchangeably and describe fluorine, chlorine, bromine or iodine.

The term “haloalkyl” describes an alkyl group as defined above, further substituted by one or more halide.

The term “sulfate” describes a —O—S(═O)₂—OR′ end group, as this term is defined hereinabove, or an —O—S(═O)₂—O— linking group, as these phrases are defined hereinabove, where R′ is as defined hereinabove.

The term “thiosulfate” describes a —O—S(═S)(═O)—OR′ end group or a —O—S(═S)(═O)—O-linking group, as these phrases are defined hereinabove, where R′ is as defined hereinabove.

The term “sulfite” describes an —O—S(═O)—O—R′ end group or a —O—S(═O)—O— group linking group, as these phrases are defined hereinabove, where R′ is as defined hereinabove.

The term “thiosulfite” describes a —O—S(═S)—O—R′ end group or an —O—S(═S)—O— group linking group, as these phrases are defined hereinabove, where R′ is as defined hereinabove.

The term “sulfinate” describes a —S(═O)—OR′ end group or an —S(═O)—O— group linking group, as these phrases are defined hereinabove, where R′ is as defined hereinabove.

The term “sulfoxide” or “sulfinyl” describes a —S(═O)R′ end group or an —S(═O)— linking group, as these phrases are defined hereinabove, where R′ is as defined hereinabove.

The term “sulfonate” or “sulfonyl” describes a —S(═O)₂—R′ end group or an —S(═O)₂-linking group, as these phrases are defined hereinabove, where R′ is as defined herein.

The term “S-sulfonamide” describes a —S(═O)₂—NR′R″ end group or a —S(═O)₂—NR′-linking group, as these phrases are defined hereinabove, with R′ and R″ as defined herein.

The term “N-sulfonamide” describes an R'S(═O)₂—NR″— end group or a —S(═O)₂—NR′-linking group, as these phrases are defined hereinabove, where R′ and R″ are as defined herein.

The term “disulfide” refers to a —S—SR′ end group or a —S—S— linking group, as these phrases are defined hereinabove, where R′ is as defined herein.

The term “carbonyl” or “carbonate” as used herein, describes a —C(═O)—R′ end group or a —C(═O)— linking group, as these phrases are defined hereinabove, with R′ as defined herein.

The term “thiocarbonyl” as used herein, describes a —C(═S)—R′ end group or a —C(═S)— linking group, as these phrases are defined hereinabove, with R′ as defined herein.

The term “oxo” as used herein, describes a (═O) group, wherein an oxygen atom is linked by a double bond to the atom (e.g., carbon atom) at the indicated position.

The term “thiooxo” as used herein, describes a (═S) group, wherein a sulfur atom is linked by a double bond to the atom (e.g., carbon atom) at the indicated position.

The term “oxime” describes a ═N—OH end group or a ═N—O— linking group, as these phrases are defined hereinabove.

The term “hydroxyl” describes a —OH group.

The term “alkoxy” describes both an —O-alkyl and an —O-cycloalkyl group, as defined herein.

The term “aryloxy” describes both an —O-aryl and an —O-heteroaryl group, as defined herein.

The term “thiohydroxy” describes a —SH group.

The term “thioalkoxy” describes both a —S-alkyl group, and a —S-cycloalkyl group, as defined herein.

The term “thioaryloxy” describes both a —S-aryl and a —S-heteroaryl group, as defined herein.

The “hydroxyalkyl” is also referred to herein as “alcohol”, and describes an alkyl, as defined herein, substituted by a hydroxy group.

The term “cyano” describes a —C≡N group.

The term “isocyanate” describes an —N═C═O group.

The term “isothiocyanate” describes an —N═C═S group.

The term “nitro” describes an —NO₂ group.

The term “acyl halide” describes a —(C═O)R″″ group wherein R″″ is halide, as defined hereinabove.

The term “azo” or “diazo” describes an —N═NR′ end group or an —N═N— linking group, as these phrases are defined hereinabove, with R′ as defined hereinabove.

The term “peroxo” describes an —O—OR′ end group or an —O—O— linking group, as these phrases are defined hereinabove, with R′ as defined hereinabove.

The term “carboxylate” as used herein encompasses C-carboxylate and O-carboxylate.

The term “C-carboxylate” describes a —C(═O)—OR′ end group or a —C(═O)—O— linking group, as these phrases are defined hereinabove, where R′ is as defined herein.

The term “O-carboxylate” describes a —OC(═O)R′ end group or a —OC(═O)— linking group, as these phrases are defined hereinabove, where R′ is as defined herein.

A carboxylate can be linear or cyclic. When cyclic, R′ and the carbon atom are linked together to form a ring, in C-carboxylate, and this group is also referred to as lactone. Alternatively, R′ and O are linked together to form a ring in O-carboxylate. Cyclic carboxylates can function as a linking group, for example, when an atom in the formed ring is linked to another group.

The term “thiocarboxylate” as used herein encompasses C-thiocarboxylate and O-thiocarboxylate.

The term “C-thiocarboxylate” describes a —C(═S)—OR′ end group or a —C(═S)—O— linking group, as these phrases are defined hereinabove, where R′ is as defined herein.

The term “O-thiocarboxylate” describes a —OC(═S)R′ end group or a —OC(═S)— linking group, as these phrases are defined hereinabove, where R′ is as defined herein.

A thiocarboxylate can be linear or cyclic. When cyclic, R′ and the carbon atom are linked together to form a ring, in C-thiocarboxylate, and this group is also referred to as thiolactone. Alternatively, R′ and O are linked together to form a ring in O-thiocarboxylate. Cyclic thiocarboxylates can function as a linking group, for example, when an atom in the formed ring is linked to another group.

The term “carbamate” as used herein encompasses N-carbamate and O-carbamate.

The term “N-carbamate” describes an R″OC(═O)—NR′— end group or a —OC(═O)—NR′-linking group, as these phrases are defined hereinabove, with R′ and R″ as defined herein.

The term “O-carbamate” describes an —OC(═O)—NR′R″ end group or an —OC(═O)—NR′— linking group, as these phrases are defined hereinabove, with R′ and R″ as defined herein.

A carbamate can be linear or cyclic. When cyclic, R′ and the carbon atom are linked together to form a ring, in O-carbamate. Alternatively, R′ and O are linked together to form a ring in N-carbamate. Cyclic carbamates can function as a linking group, for example, when an atom in the formed ring is linked to another group.

The term “carbamate” as used herein encompasses N-carbamate and O-carbamate.

The term “thiocarbamate” as used herein encompasses N-thiocarbamate and O-thiocarbamate.

The term “O-thiocarbamate” describes a —OC(═S)—NR′R″ end group or a —OC(═S)—NR′— linking group, as these phrases are defined hereinabove, with R′ and R″ as defined herein.

The term “N-thiocarbamate” describes an R″OC(═S)NR′— end group or a —OC(═S)NR′-linking group, as these phrases are defined hereinabove, with R′ and R″ as defined herein.

Thiocarbamates can be linear or cyclic, as described herein for carbamates.

The term “dithiocarbamate” as used herein encompasses S-dithiocarbamate and N-dithiocarbamate.

The term “S-dithiocarbamate” describes a —SC(═S)—NR′R″ end group or a —SC(═S)NR′— linking group, as these phrases are defined hereinabove, with R′ and R″ as defined herein.

The term “N-dithiocarbamate” describes an R″SC(═S)NR′— end group or a —SC(═S)NR′-linking group, as these phrases are defined hereinabove, with R′ and R″ as defined herein.

The term “urea”, which is also referred to herein as “ureido”, describes a —NR′C(═O)—NR″R′″ end group or a —NR′C(═O)—NR″— linking group, as these phrases are defined hereinabove, where R′ and R″ are as defined herein and R′″ is as defined herein for R′ and R″.

The term “thiourea”, which is also referred to herein as “thioureido”, describes a —NR′—C(═S)—NR″R′″ end group or a —NR′—C(═S)—NR″— linking group, with R′, R″ and R′″ as defined herein.

The term “amide” as used herein encompasses C-amide and N-amide.

The term “C-amide” describes a —C(═O)—NR′R″ end group or a —C(═O)—NR′— linking group, as these phrases are defined hereinabove, where R′ and R″ are as defined herein.

The term “N-amide” describes a R′C(═O)—NR″— end group or a R′C(═O)—N— linking group, as these phrases are defined hereinabove, where R′ and R″ are as defined herein.

An amide can be linear or cyclic. When cyclic, R′ and the carbon atom are linked together to form a ring, in C-amide, and this group is also referred to as lactam. Cyclic amides can function as a linking group, for example, when an atom in the formed ring is linked to another group.

The term “guanyl” also describes a R′R″NC(═N)— end group or a —R′NC(═N)— linking group, as these phrases are defined hereinabove, where R′ and R″ are as defined herein.

The term “guanidine” also describes a —R′NC(═N)—NR″R′″ end group or a —R′NC(═N)— NR″— linking group, as these phrases are defined hereinabove, where R′, R″ and R′″ are as defined herein.

The term “hydrazine” describes a —NR′—NR″R′″ end group or a —NR′—NR″— linking group, as these phrases are defined hereinabove, with R′, R″, and R′″ as defined herein.

As used herein, the term “hydrazide” describes a —C(═O)—NR′—NR″R′″ end group or a —C(═O)—NR′—NR″— linking group, as these phrases are defined hereinabove, where R′, R″ and R′″ are as defined herein.

As used herein, the term “thiohydrazide” describes a —C(═S)—NR′—NR″R′″ end group or a —C(═S)—NR′—NR″— linking group, as these phrases are defined hereinabove, where R′, R″ and R′″ are as defined herein.

Herein throughout, the term “acyl” describes a —C(═O)—R group, wherein R is as described herein.

Herein throughout, the term “acyl” describes a —C(═O)—R group, with R being a substituted or unsubstituted alkyl, cycloalkyl, aryl, alkaryl, a hydrocarbon chain, or hydrogen.

The term “ether” as used herein described a —R′—O—R″ end group or a —R′—O—R″— linking group, as these phrases are described hereinabove, where R′ and R″ are as defined herein.

The term “thioether” as used herein described a —R′—S—R″ end group or a —R′—S—R″-linking group, as these phrases are described hereinabove, where R′ and R″ are as defined herein.

According to some of any of the embodiments described herein, any of the compounds prepared or provided according to the present embodiments can be in a form of a pharmaceutically acceptable salt thereof.

As used herein, the phrase “pharmaceutically acceptable salt” refers to a charged species of the parent compound and its counter-ion, which is typically used to modify the solubility characteristics of the parent compound and/or to reduce any significant irritation to an organism by the parent compound, and/or to improve its stability, while not abrogating the biological activity and properties of the administered compound. A pharmaceutically acceptable salt of a compound as described herein can alternatively be formed during the synthesis of the compound, e.g., in the course of isolating the compound from a reaction mixture or re-crystallizing the compound.

In the context of some of the present embodiments, a pharmaceutically acceptable salt of the compounds described herein may optionally be an acid addition salt comprising at least one basic (e.g., an amine-containing group) group of the compound which is in a positively charged form (e.g., wherein the basic group is protonated), in combination with at least one counter-ion, derived from the selected base, that forms a pharmaceutically acceptable salt; and/or at least one acidic group of the compound which is in a negatively charged form (e.g., de-protonated) in combination with at least one counter-ion, derived from the selected base, that forms a pharmaceutically acceptable salt. Oxonium positively charged ions and a counter anion are also contemplated.

The acid addition salts of the compounds described herein may therefore be complexes formed between one or more basic groups of the compound and one or more equivalents of an acid.

Depending on the stoichiometric proportions between the charged group(s) in the compound and the counter-ion in the salt, the acid additions salts can be either mono-addition salts or poly-addition salts.

The phrase “mono-addition salt”, as used herein, refers to a salt in which the stoichiometric ratio between the counter-ion and charged form of the compound is 1:1, such that the addition salt includes one molar equivalent of the counter-ion per one molar equivalent of the compound.

The phrase “poly-addition salt”, as used herein, refers to a salt in which the stoichiometric ratio between the counter-ion and the charged form of the compound is greater than 1:1 and is, for example, 2:1, 3:1, 4:1 and so on, such that the addition salt includes two or more molar equivalents of the counter-ion per one molar equivalent of the compound.

An example, without limitation, of a pharmaceutically acceptable salt would be an ammonium cation or guanidinium cation and an acid addition salt thereof.

The acid addition salts may include a variety of organic and inorganic acids, such as, but not limited to, hydrochloric acid which affords a hydrochloric acid addition salt, hydrobromic acid which affords a hydrobromic acid addition salt, acetic acid which affords an acetic acid addition salt, ascorbic acid which affords an ascorbic acid addition salt, benzenesulfonic acid which affords a besylate addition salt, camphorsulfonic acid which affords a camphorsulfonic acid addition salt, citric acid which affords a citric acid addition salt, maleic acid which affords a maleic acid addition salt, malic acid which affords a malic acid addition salt, methanesulfonic acid which affords a methanesulfonic acid (mesylate) addition salt, naphthalenesulfonic acid which affords a naphthalenesulfonic acid addition salt, oxalic acid which affords an oxalic acid addition salt, phosphoric acid which affords a phosphoric acid addition salt, toluenesulfonic acid which affords a p-toluenesulfonic acid addition salt, succinic acid which affords a succinic acid addition salt, sulfuric acid which affords a sulfuric acid addition salt, tartaric acid which affords a tartaric acid addition salt and trifluoroacetic acid which affords a trifluoroacetic acid addition salt. Each of these acid addition salts can be either a mono-addition salt or a poly-addition salt, as these terms are defined herein.

The present embodiments further encompass any enantiomers, diastereomers, prodrugs, solvates, hydrates and/or pharmaceutically acceptable salts of the compounds described herein.

As used herein, the term “enantiomer” refers to a stereoisomer of a compound that is superposable with respect to its counterpart only by a complete inversion/reflection (mirror image) of each other. Enantiomers are said to have “handedness” since they refer to each other like the right and left hand. Enantiomers have identical chemical and physical properties except when present in an environment which by itself has handedness, such as all living systems. In the context of the present embodiments, a compound may exhibit one or more chiral centers, each of which exhibiting an R- or an S-configuration and any combination, and compounds according to some embodiments of the present invention, can have any their chiral centers exhibit an R- or an S-configuration.

The term “diastereomers”, as used herein, refers to stereoisomers that are not enantiomers to one another. Diastereomerism occurs when two or more stereoisomers of a compound have different configurations at one or more, but not all of the equivalent (related) stereocenters and are not mirror images of each other. When two diastereoisomers differ from each other at only one stereocenter they are epimers. Each stereo-center (chiral center) gives rise to two different configurations and thus to two different stereoisomers. In the context of the present invention, embodiments of the present invention encompass compounds with multiple chiral centers that occur in any combination of stereo-configuration, namely any diastereomer.

The term “prodrug” refers to an agent, which is converted into the active compound (the active parent drug) in vivo. Prodrugs are typically useful for facilitating the administration of the parent drug. They may, for instance, be bioavailable by oral administration whereas the parent drug is not. A prodrug may also have improved solubility as compared with the parent drug in pharmaceutical compositions. Prodrugs are also often used to achieve a sustained release of the active compound in vivo. An example, without limitation, of a prodrug would be a compound of the present invention, having one or more carboxylic acid moieties, which is administered as an ester (the “prodrug”). Such a prodrug is hydrolyzed in vivo, to thereby provide the free compound (the parent drug). The selected ester may affect both the solubility characteristics and the hydrolysis rate of the prodrug.

The term “solvate” refers to a complex of variable stoichiometry (e.g., di-, tri-, tetra-, penta-hexa-, and so on), which is formed by a solute (the compound of the present invention) and a solvent, whereby the solvent does not interfere with the biological activity of the solute. Suitable solvents include, for example, ethanol, acetic acid and the like.

The term “hydrate” refers to a solvate, as defined hereinabove, where the solvent is water.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

### Materials and Experimental Methods

Cell Lines, Growth Conditions and Reagents:

MDA-MB-231, A375, G361, SK-MEL1 and HMEC-1 were all originally obtained from ATCC. All adherent cell lines (unless otherwise specified) were maintained in DMEM (Thermo Fisher Scientific) supplemented with 10% FBS (Thermo Fisher Scientific), 100 U/ml penicillin and 100 μg/ml streptomycin (Thermo Fisher Scientific).

HMEC-1 cell line was cultured in MCDB131 (Thermo Fisher Scientific) supplemented with 10 ng/ml Epidermal Growth Factor (EGF), 1 μg/ml Hydrocortisone, 10 mM Glutamine and 10% (v/v) FBS.

MC38-hPD-L1 cell line was purchased from genOway and maintained according to the manufacturer's instructions.

All cell lines were routinely screened for mycoplasma contamination.

Patient-derived cells were maintained in RMPI-1640 supplemented with 10% FBS (Thermo Fisher Scientific), 100 U/ml penicillin and 100 μg/ml streptomycin (Thermo Fisher Scientific).

PD-1/PD-L1 binding assay kit was purchased from Cisbio Assays.

Paraformaldehyde 4% (v/v), PD-L1 recombinant human protein and Invitrogen SYPRO® orange (5000× solution) was purchased from ThermoFisher Scientific.

MTT® reagent, bovine serum albumin (BSA) and Ficoll® Paque Plus was purchased from Sigma-Aldrich.

BMS202 was purchased from Selleckchem (Houston, USA).

Human anti-PD-L1 and anti-CD28 monoclonal antibodies were acquired from BioXcell.

Recombinant human IFN-γ and recombinant human IL-2 were purchased from Peprotech. Fluorochrome-labeled antibodies for flow cytometry were purchased from Miltenyi Biotech, Biolegend and eBiosciense.

Animal Studies:

Male C57BL/6N-Pdcd1tm1^{(huPDCD1-ICP11)Geno} humanized for PD-1 (6 weeks old) were purchased from genOway and housed in an animal facility.

Male C57BL/6N humanized PD-1 mice were implanted with 1×10⁶ MC38 cells expressing humanized PD-L1 (MC38-hPD-L1) subcutaneously in the right flank. Twelve days later, when tumor volumes reached approx. 60 mm³ (40-110 mm³) as measured by digital caliper, animals were randomized into the three treatment groups (n=6 per group). Compound 69 was administered via intraperitoneal (i.p.) injection at 10 mg/kg for 10 daily doses between study days 12 and 22. Similarly, vehicle control (5% (v/v) DMSO, 30% (v/v) polyethylene glycol (PEG) 300, 5% (v/v) Tween® 80, and double distilled (dd) H₂O) was administered. The αPD-L1 antibody Atezolizumab was administered via i.p. injection at 10 mg/kg thrice weekly between study days 12 and 22. The tumor size and body weight were measured every 3 days. The tumor volume was determined by X²Y0.5 (X, small diameter; Y, large diameter).

At day 30, the mice were euthanized, and MC38 tumors and spleens were collected. Tumor and spleen single-cell suspensions were obtained by mechanical disruption of the tissues. Tumors were further digested in RPMI medium with 0.5% BSA, 0.1% collagenase type II (LS004177, Worthington), 0.1% dispase (LS02109, Worthington), and DNase (LS002007, Worthington) for 1 hour at 37° C. The suspension was then filtered through a 70 m filter (BD Biosciences) to remove the debris. ACK lysing buffer was added to tumor and spleen single-cell suspensions for red blood cell lysis. The obtained single-cell suspensions were then stained with fluorochrome-labelled antibodies and analyzed using a Cytek Aurora (Cytek) and FlowJo software (TreeStar).

Virtual Screening Library:

A library of commercial compounds was generated from National Cancer Institute (www(dot)cancer(dot)gov), Enamine (www(dot)enamine(dot)net), Specs (www(dot)specs(dot)net), Mu.Ta.Lig Chemoteca (www(dot)mutalig(dot)eu), MMV (www(dot)mmv(dot)org) and inhouse compounds. NCI collection of compound structures was built and is maintained by the Developmental Therapeutics Program, Division of Cancer Treatment and Diagnosis of the National Cancer Institute, National Institute of Health (Bethesda, MD, USA).

Briefly, compounds were prepared using the Molecular Operating Environment (MOE) 20180101 software package [Molecular Operating Environment (MOE) 2013.08], protonated (at pH=7.4 and 310 K), and partial charges were assigned using Amberl0 EHT force field. Compounds were further energy-minimized.

Three-dimensional (3D) PD-L1 structure (PDBID: 5J89) retrieved from Protein Data Bank (www(dot)rcsb(dot)org) was used for structure-based virtual screening following protein preparation in Molecular Operating Environment (MOE) 20180101. In short, this included water (no crystallographic waters were found to be structurally important) and ligands removal, in addition to hydrogen atoms and optimization of the hydrogen bonding network.

In Silico Virtual Screening:

Structure-based virtual screening (VS) using molecular docking studies was performed. Genetic Optimization for Ligand Docking (GOLD) 5.2.0 suite of programs was used to analyze the binding conformations [Jones, G., Willet, P., Glen, R. C., Leach, A. R. & Taylor, R. Development and Validation of a Genetic Algorithm for Flexible Docking. J. Mol. Biol. 267, 727-748 (1997)]. PD-L1 (PDBID: 5J89) structure was validated for virtual screening (VS) by performing molecular docking of the crystallographic ligand into the protein active site. All default options were used, _{A}Tyr56 as the center of the binding pocket with 10 Å radius, and 1000 genetic algorithm (GA) runs.

Initially, VS was performed with speed-up settings, using ChemPLP fitness scoring function and 50 GA runs. The top 1000 highest ranked compounds in VS were selected for posterior molecular docking studies with a more accurate modality (GoldScore fitness scoring function with 500 GA runs). The pocket was visually inspected, and the selection of compounds was based on (i) score, (ii) appropriate pocket fitting and (iii) interactions with the surrounding residues. Compounds were subsequently filtered by FAF-Drugs4 tool using the following chemical property ranges that correspond to the Lipinski's rule of five for enhanced drug-likeness: MW 300-550, hydrogen-bond donors 0-5, hydrogen-bond acceptors 1-10, Rotatable bonds 0-10 and Log P 0-5.5.

Chemical Compounds:

Hit compounds were obtained from National Cancer Institute (NCI). Compounds were characterized by nuclear magnetic resonance (NMR). ¹H-NMR spectra were recorded on a Bruker Avance 300 MHz NMR spectrometer using deuterated solvents. The chemical shift data was obtained as 6H in ppm and referenced against the deuterated solvent used. Coupling constants were determined using MestreNova and the values are quoted in Hz. The NMR correspond to the hits identified on cell-based experiments.

To test compounds in cellular assays, these small molecule drugs were dissolved at 10 mM in DMSO (stock solution). All compounds were diluted in DMSO to the required concentration immediately before testing (DMSO content was less than 1% in final media).

Htrf® Assay:

PD-1/PD-L1 binding assay kit was reconstituted according to the supplier protocols (Cisbio Assays). HTRF® assays were performed in white 96-well low volume plates (Cisbio Assays) with a final volume of 20 μL comprising 2.0 μl of compound (100 μM), 4.0 μl of Tag1-PD-L1 (5 nM) and 4.0 μl of Tag2-PD-1 (50 nM). After 10 minutes of incubation at room temperature, detection reagents were added: 10 μL of pre-mixed anti-Tag1-Europium and anti-Tag2-XL665. HTRF® signal was measured after 2 hours using a microplate reader (POLARstar Omega, BMG LABTECH Ltd.) using the following setup: excitation 337 nm, emissions 620 nm and 665 nm. Dilution buffer and BMS-202 were used as negative and positive controls, respectively. Results were analyzed with a two-wavelength signal ratio: [intensity (665 nm)/intensity (620 nm)]×10⁴ (HTRF® Ratio). The normalized HTRF® ratio was calculated as follow: [(compound signal)−(min signal)]/[(max signal)−(min signal)]×100, where ‘max signal’ is the signal ratio with PD-1/PD-L1 and ‘min signal’ the signal ratio without PD-1.

For the first screening assay, each chemical was tested in duplicate. True hits were tested in three independent experiments.

To access the binding properties towards each compound, the HTRF® assay was performed in the presence of increasing compound concentrations (0.0001-100 μM) or 1% (v/v) DMSO (vehicle control). Half-maximum inhibition by inhibitory compounds (IC₅₀ values) were calculated using log (inhibitor) vs. normalized response function of GraphPad Prism software (v.7.03).

DSF:

DSF was performed in a C1000 Touch thermal cycler equipped with a CFX96 optical reaction module (Bio Rad). For all fluorescence measurements, samples contained recombinant human PD-L1 (ThermoFisher) at 500 μg/ml in phosphate buffered saline (PBS), pH 7.4, 5% (m/v) Mannitol, 5% (m/v) Trehalose, 0.02% (v/v) Tween® 80, 2.5-fold SYPRO® Orange, 1% (v/v) DMSO (Sigma-Aldrich) and 100 μM of each compound. The PCR plate was sealed with Optical-Quality Sealing Tape (Bio-Rad) and centrifuged at 300 g for 5 minutes. The DSF assay was carried out by increasing the temperature from 20 to 90° C., with a 1 second hold time every 0.2° C. and fluorescence acquisition using the FRET channel, after an initial incubation step of 10 minutes at 20° C. Control experiments in the absence of DMSO and/or compounds were routinely performed in each microplate.

Data were processed using CFX Manager Software V3.0 (Bio-Rad) and the GraphPad Prism 7. Temperature scan curves were fitted to a dose-response sigmoid function (Boltzmann equation) and the melting temperature (Tm values) were obtained from the midpoint of the transition. Normalization of the RFU was also performed to prevent distraction due to different maximum and minimum values upon compounds treatment. The RFU values from different data sets were converted to a common scale 0-1, where 0 represents the fluorescence of the native protein and 1 the fluorescence of the denatured protein. To monitor the binding properties towards each compound, DSF assays were run in the absence and presence of 100 μM compounds using 1% (v/v) DMSO as vehicle control.

WaterLOGSY NMR:

WaterLOGSY NMR was performed based on a previously described assay [Gossert, A. D. & Jahnke, W. Prog. Nucl. Magn. Reson. Spectrosc. 97, 82-125 (2016); and Raingeval, C. et al. J. Enzyme Inhib. Med. Chem. 34, 1218-1225 (2019)] using 150 μM of BMS202 or Compound 69, each in the presence and absence of 5 μM PD-L1.

NMR experiments were performed using a NMR Bruker AVANCE-TM 600 MHz Spectrometer with a 5 mm BBO probe, the acquisition temperature was set at 25° C. For WaterLOGSY experiments, 0.15 mM of ligand (from a 10 mM stock in DMSO-d6) were added to 5 μM PD-L1 samples in 10 mM sodium phosphate, 25 mM NaCl, pH 7.6 with 10% D₂O, in a protein/ligand ratio of 1:30, optimal for the WaterLOGSY experiments. For each compound, samples were prepared with and without protein. For each sample, 1D 1H and WaterLOGSY experiments were acquired. A total of 16K-points were used for a sweep width of 16 ppm in both experiments. For the 1D ¹H experiments, 256 scans were accumulated. A total of 528 scans were accumulated for the WaterLOGSY experiment. Spectra were acquired and processed with Topspin 4.1 (Bruker Biospin), and MNova software (MestReNova, v14.2.0).

Cell Viability:

For the MTT assays, cells were seeded in 96-multi-well plate at a concentration of 7,000 cells per well. Six hours after seeding, cells were treated with the compounds and DMSO as a negative control. Forty-eight hours after the addition of the compounds, 20 μl of MTT (Sigma-Aldrich) dissolved in 5 mg/ml of PBS were added to each well and incubated for 2 hours at 37° C. Solutions were removed, and 100 μl of DMSO were added to each well and gently mixed on a shaker. The absorbance of control and treated wells was read against a DMSO blank at 570 nm using an Epoch micro-plate reader (Biotek). Each hit compound was tested in three replicates, in three independent experiments.

PD-1/PD-L1 Inhibition on Human Melanoma and Breast Cancer Cell Lines:

Cells (0.1×10⁶ cells) were tested for PD-1/PD-L1 inhibition by co-incubation of hit compounds, DMSO (background), BMS-202 (Selleckchem) and anti-human PD-L1 (BioxCell, Clone 29E.2A3) (positive controls) for 72 hours in 2 ml DMEM medium. Non-confluent cell cultures were scraped into single-cell suspension, washed with PBS, and counted. Cells were subsequently stained with PD-1 fluorescent proxy for 30 minutes at 4° C., washed twice and resuspended in FACS buffer. Cells were analyzed using BD LSRFortessa (BD Biosciences) and data analyzed with FlowJo software for Mac (FlowJo, LLC 2013-2016). Mean fluorescence intensity (MFI) was derived from each sample. The PD-1/PD-L1 inhibition in cells was measured by a decrease in MFI relative to background.

Isolation and Culture of Patient-Derived Peripheral Blood Mononuclear Cells (PBMC):

The PBMC fraction was isolated from peripheral blood of patients by Ficoll-Paque (Sigma-Aldrich) density gradient separation and cryopreserved until later use. The obtained single-cell suspensions were then stained with fluorochrome-labelled antibodies and analyzed using an LSR Fortessa (BD Biosciences) and FlowJo software.

Isolation and Culture of Patient-Derived Tumor Cells:

Tumor single-cell suspensions were obtained by mechanical disruption of the tissues and enzymatic digestion in PBS with 0.5% (m/v) BSA, 0.1% (m/v) collagenase type II (LS004177, Worthington) and 0.1% (m/v) dispase (LS02109, Worthington) for 1 hour at 37° C. After digestion, the suspension was filtered through a 70 m filter (BD Biosciences) to remove the debris. The obtained single-cell suspensions were then stained with fluorochrome-labelled antibodies and analyzed using an LSR Fortessa (BD Biosciences) and FlowJo software.

Patient-derived tumor cells were maintained in RMPI-1640 (Thermo Fisher Scientific) supplemented with 10 FBS, 100 U/ml penicillin and 100 μg/ml streptomycin (Tumor media). The peripheral blood mononuclear cells (PBMC) were maintained in RMPI-1640 (Thermo Fisher Scientific) supplemented with 10% FBS, 100 U/ml penicillin, 100 μg/ml streptomycin and 150 U/ml IL-2 (Peprotech) (T cell media).

Tumor—Lymphocyte Co-Culture:

One day before co-culture, PBMC were re-suspended in RPMI-1640 supplemented with 150 U/ml IL-2 (Peprotech) and cultured overnight at 37° C. Prior to co-culture, tumor cells were stimulated overnight with 200 ng/ml human recombinant IFN-γ (Peprotech). The next day, tumor cells were scraped to single cells and re-suspended in RPMI. PBMC were seeded at a density of 0.1⁶ cells/well and stimulated with single cell-dissociated tumor cells at a 2:1 Effector:Target ratio. Co-cultures were performed in the presence of 150 U/ml IL-2 (Peprotech), 5 μg/ml of anti-CD28 (BioxCell) and αPD-L1 blocking antibody or small-molecule inhibitor for 72 hours. The cells were then stained with fluorochrome-labeled antibodies and analyzed using an LSR Fortessa (BD Biosciences) and FlowJo software.

3D Patient-Derived Tumor Spheroids—Lymphocyte Co-Culture:

After isolation of tumor cells, cells were washed and cultured for up to 3 days. This allowed the isolation of adherent cells that were then scrapped and seeded in Nunclon Sphera 96-well plate (Thermo Fisher Scientific) to create a unique uniform spheroid in each well. Spheroids were ready on day 2-5. Thereafter, spheroids were embedded in reduced growth Matrigel (Corning) with PBMC (1:100) and were left untreated or were treated with the small-molecule inhibitor Compound 69 or αPD-L1, dissolved in the appropriate cell culture medium.

Immunofluorescence (IF):

3D co-culture was fixed with 4% paraformaldehyde and blocked by PBS with 2% (m/v) BSA. Thereafter, the samples were incubated with CD8 (MA5-16345—Invitrogen) (1:50) in PBS with 1% (v/v) Tween® 20 for 18 hours at 4° C. Then, 4 μg/ml of secondary antibody (A-11008, Invitrogen) were added, 3D spheroids were stained with Hoechst 33342 (10 μg/ml) for 20 minutes. Finally, 3D spheroid invasion was visualized using a Leica DMi8—CS inverted microscope with Leica LAS X software (Leica Microsystems Inc.). The different z-stacks were merged and analyzed by Fiji-ImageJ.

Flow Cytometry:

All staining was performed in FACS buffer (0.5% (m/v) BSA, 2 mM EDTA in PBS). Stains were performed for 30 minutes at 4° C. and followed by two washes with FACS buffer before fixation in 2% PFA in PBS. This was followed by permeabilization with the Inside stain kit (Miltenyi Biotec), according to the manufacturer's protocol. Cells were stained with the viability dye Live/Dead yellow (Thermo Fisher Scientific) according to the manufacturer's protocol. Samples were acquired on a BD LSRFortessa (BD Biosciences). Analysis was performed using FlowJo (FlowJo LLC).

To analyze cell surface marker expression, cells were collected, washed with PBS and stained with the viability dye Live/Dead Aqua. Cells were then washed with FACS buffer and stained for surface markers: CD45 (APC-Vio® 770, clone REA747), CD3 (PerCP Cy5.5, clone OKT3), CD4 (FITC, clone REA623), CD8 (VioBlue®, clone REA734), CD107a (PE-Cy5, clone eBioH4A3), CD279 (PE, clone REA1165) and CD274 (BV711®, clone 29E.2A3).

To analyze intracellular cytokines, cells were collected, washed with PBS, and stained with the viability dye Live/Dead Yellow. Cells were then washed with FACS buffer and stained for surface markers. Cells were further fixed and permeabilized and stained for the intracellular cytokines IFN-γ (PE-Vio® 770, clone REA600) and TNFα (APC, clone REA656).

Statistics:

Samples sizes and statistical tests are as defined in the respective data and accompanying Figures. Results for technical replicates are presented as mean±SD. Statistical significance between conditions was calculated using Student's t-test (two-tailed) when comparing two groups, and one-way analysis of variance followed by Dunnett's or Tukey's post hoc analysis when comparing more than two. All statistical calculations were performed using the software package GraphPad Prism (v.7.03).

### Example 1

### Identification of PD-1/PD-L1 Small-Molecule Inhibitors

In Silico Screening:

The discovery of small molecules as immune checkpoint inhibitors has been suggested as a promising approach to overcome the limitations of currently available therapeutics. However, they are technically difficult to identify and assess. Together with a challenging design, the limited structural elucidation of the targets has been compromising the development of PD-1/PD-L1 small-molecule inhibitors.

Before 2015, no human PD-1/PD-L1 X-ray structure was resolved and the murine form does not allow the assessment of the extent of plasticity or interactions established with the PD-L1 [Zak, K. M. et al. 2015, supra]. In the last years, several human PD-1 and PD-L1 X-ray structures have been resolved to expose the murine/human structural differences within the binding modes between proteins as well as the plasticity in the complex formation [Zak, K. M. et al. supra].

The advances in PD-1/PD-L1 structural characterization anticipated a substantial progress on the development of small-molecule inhibitors. However, the design of inhibitors directly targeting the PD-1/PD-L1 interaction interface has been limited by the larger, hydrophobic, and flat interface between proteins without deep binding pockets.

Recently, different X-ray structures of PD-L1 with a class of small-molecule inhibitors have been resolved [Guzik, K. et al. J. Med. Chem. 1, acs.jmedchem.7b00293 (2017); Zak, K. M. et al. 2016, supra].

Previous studies have reported biphenyl-based compounds, known as BMS (Bristol-Myers Squibb) compounds as the first PD-1/PD-L1 small-molecule inhibitors [Abdel-Magid, A. F. ACS Med. Chem. Lett. 6, 489-490 (2015); Guzik, K. et al. J. Med. Chem. 1, acs.jmedchem.7b00293 (2017); Skalniak, L. et al. 2017, supra]. However, these were reported as compounds with poor drug-like properties [Zak, K. M. et al. 2016, supra].

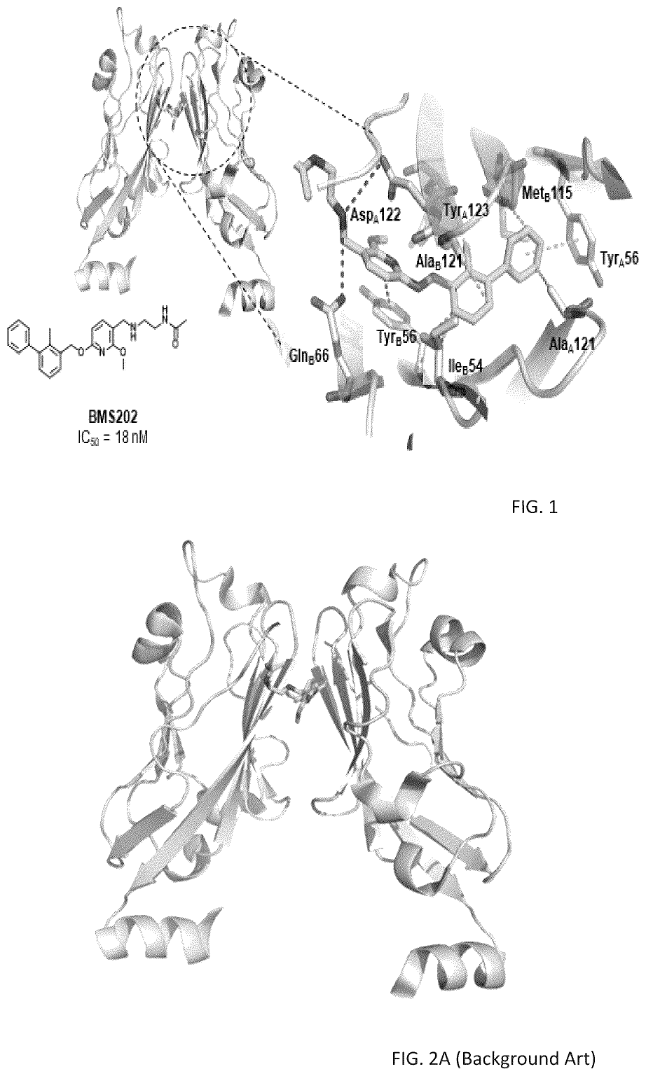

In general, and as shown in FIG. 1, these inhibitors bind to PD-L1 leading to a deep cylindrical, hydrophobic pocket created by the interface of two monomers.

Aiming for the discovery of new PD-1/PD-L1 inhibitors and taking into account the increasing number of the crystal structures available for this immune checkpoint, a comprehensive structure-based virtual screening (SBVS) was performed.

A crystal structure of human PD-L1 (PDB 5J89) was selected to be used in these in silico studies [Acnrcio, R. C. et al. Medchemcomm 10, 1810-1818 (2019)], as shown in Background Art FIG. 2A.

A collection of approximately 900,000 commercially available compounds from several drug repositories (e.g. NCI, Enamine, SPECS, or in house, as described hereinabove). The compounds collection was screened using molecular docking into the PD-L1 binding site. Structure-based virtual screening for the identification of PD-1/PD-L1 small-molecule inhibitors was performed as shown in FIG. 2B, beginning with pre-filtering the compounds on the basis of molecular weight (MW), followed by the screening in silico using the scoring function GoldScore of the GOLD software, followed by the visual inspection of the top-ranked compounds within the binding pocket. Finally, the compounds were uploaded into the FAF-Drugs predictor to address the administration, distribution, metabolism, excretion, and toxicity (ADMET) properties.

The first refinement was performed with speed up settings. The top ranked compounds were then subjected to exhaustive docking analyses that predicted with higher precision the corresponding binding pose and the interactions within the receptor-binding pocket. Finally, the selected compound pool was filtered by applying the Lipinski's rule of five criteria (ADMET) for enhanced drug-likeness.

Only the compounds that presented favorable binding conformations, surface complementarity with the receptor, and exhibited the important interactions with key pocket residues were retained. This approach yielded around 100 possible PD-L1 binders with chemically-diverse structures, as presented generally in FIG. 2C. The chemical structures of the identified compounds are shown in Table A hereinbelow.

Identification of Small Molecules that Inhibit PD-1/PD-L1 Interaction:

The identified compounds were tested for their capacity to inhibit the PD-1/PD-L1 interaction using in vitro functional assays.

Homogeneous time resolved fluorescence (HTRF) was used to evaluate those 94 hits identified in silico. The BMS202 inhibitor, known as a small-molecule inhibitor of PD-1/PD-L1 interaction, was used as a reference. Compounds were tested at 100 μM and the determination of the inhibition levels was based on the HTRF signal reduction. Hits were defined as compounds that inhibited at least 50% of the PD-1/PD-L1 interaction. Table A presents the compound ID, structure, molecular weight, HTF assay (if positive) and IC₅₀ value (if available).

TABLE A

| | | | HTF | |
|---|---|---|---|---|
| | | | assay | |
| | | Molecular | (100 | IC₅₀ |
| ID | STRUCTURE | Weight [g/mol] | μM) | (μM) |
| 1 | 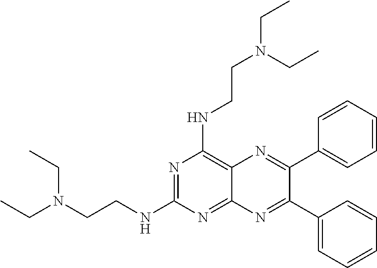 [SMILES: CCN(CC)CCNc1nc(NCCN(CC)CC)c2nc(-c3ccccc3)c(-c3ccccc3)nc2n1] | 512.6996 | + | 0.186 |
| 2 | 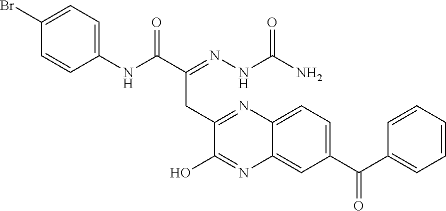 [SMILES: NC(=O)N/N=C(\Cc1nc2ccc(C(=O)c3ccccc3)cc2nc1O)C(=O)Nc1ccc(Br)cc1] | 547.3669 | | |
| 3 | 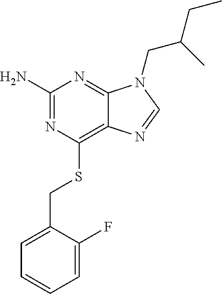 [SMILES: CCC(C)Cn1cnc2c(SCc3ccccc3F)nc(N)nc21] | 345.4369 | | |
| 4 | 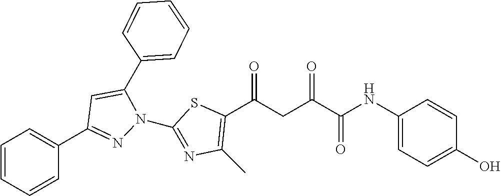 [SMILES: Cc1nc(-n2nc(-c3ccccc3)cc2-c2ccccc2)sc1C(=O)CC(=O)C(=O)Nc1ccc(O)cc1] | 522.5772 | | |
| 5 | 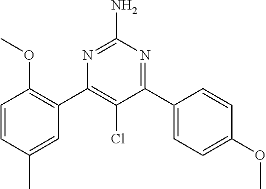 [SMILES: COc1ccc(-c2nc(N)nc(-c3cc(C)ccc3OC)c2Cl)cc1] | 378.4876 | + | 2.44 |
| 6 | 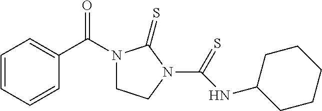 [SMILES: O=C(c1ccccc1)N1CCN(C(=S)NC2CCCCC2)C1=S] | 347.4924 | | |
| 7 | 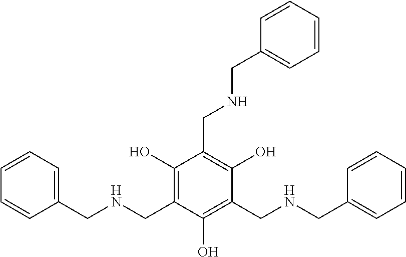 [SMILES: Oc1c(CNCc2ccccc2)c(O)c(CNCc2ccccc2)c(O)c1CNCc1ccccc1] | 483.609 | | |
| 8 | 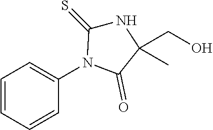 [SMILES: CC1(CO)NC(=S)N(c2ccccc2)C1=O] | 236.288 | | |
| 9 | 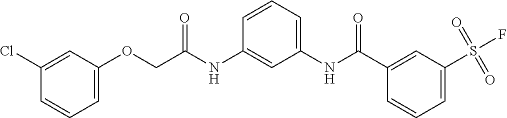 [SMILES: O=C(COc1cccc(Cl)c1)Nc1cccc(NC(=O)c2cccc(S(=O)(=O)F)c2)c1] | 462.8792 | | |
| 10 | 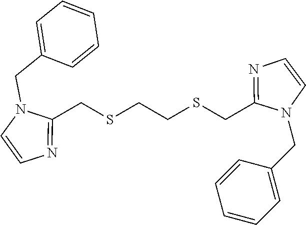 [SMILES: c1ccc(Cn2ccnc2CSCCSCc2nccn2Cc2ccccc2)cc1] | 434.6162 | | |
| 11 | 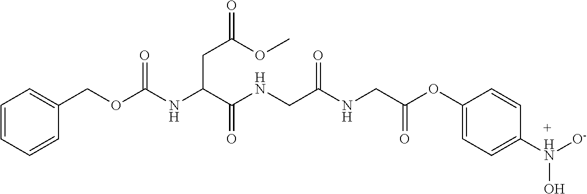 | 516.4634 | | |
| 12 | 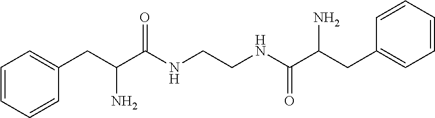 [SMILES: NC(Cc1ccccc1)C(=O)NCCNC(=O)C(N)Cc1ccccc1] | 354.451 | | |
| 13 | 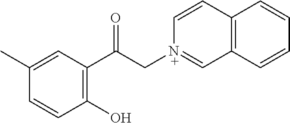 [SMILES: Cc1ccc(O)c(C(=O)C[n+]2ccc3ccccc3c2)c1] | 278.3299 | | |
| 14 | 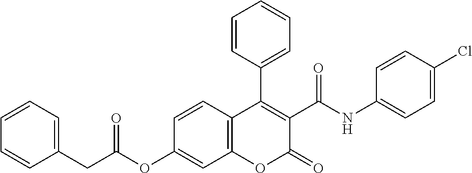 [SMILES: O=C(Cc1ccccc1)Oc1ccc2c(-c3ccccc3)c(C(=O)Nc3ccc(Cl)cc3)c(=O)oc2c1] | 509.9447 | | |
| 15 | 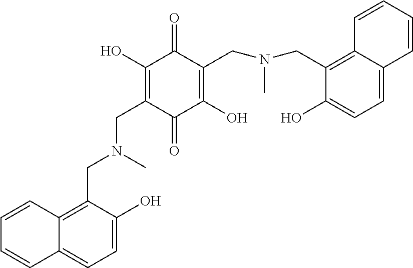 [SMILES: CN(CC1=C(O)C(=O)C(CN(C)Cc2c(O)ccc3ccccc23)=C(O)C1=O)Cc1c(O)ccc2ccccc12] | 538.5988 | | |
| 16 | 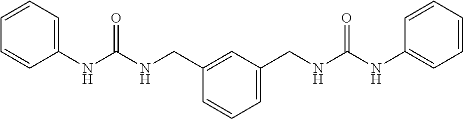 [SMILES: O=C(NCc1cccc(CNC(=O)Nc2ccccc2)c1)Nc1ccccc1] | 374.4414 | | |
| 17 | 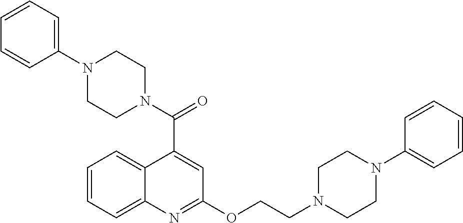 [SMILES: O=C(c1cc(OCCN2CCN(c3ccccc3)CC2)nc2ccccc12)N1CCN(c2ccccc2)CC1] | 521.6608 | | |
| 18 | 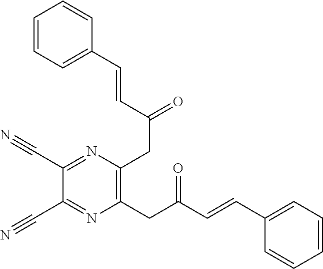 [SMILES: N#Cc1nc(CC(=O)/C=C/c2ccccc2)c(CC(=O)/C=C/c2ccccc2)nc1C#N] | 418.4538 | + | 0.19 |
| 19 | 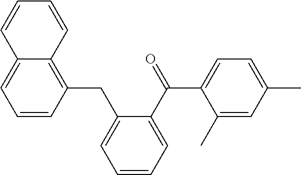 [SMILES: Cc1ccc(C(=O)c2ccccc2Cc2cccc3ccccc23)c(C)c1] | 350.4592 | | |
| 20 | 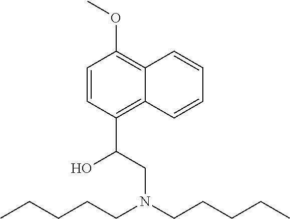 [SMILES: CCCCCN(CCCCC)CC(O)c1ccc(OC)c2ccccc12] | 357.535 | | |
| 21 | 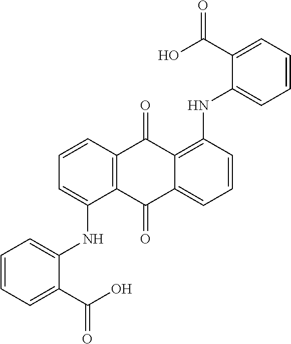 [SMILES: O=C(O)c1ccccc1Nc1cccc2c1C(=O)c1cccc(Nc3ccccc3C(=O)O)c1C2=O] | 478.46 | | |
| 22 | 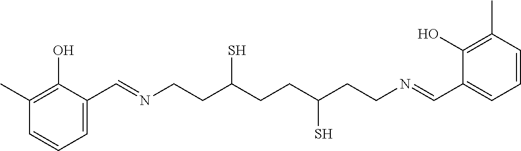 [SMILES: Cc1cccc(/C=N/CCC(S)CCC(S)CC/N=C/c2cccc(C)c2O)c1O] | 444.649 | | |
| 23 | 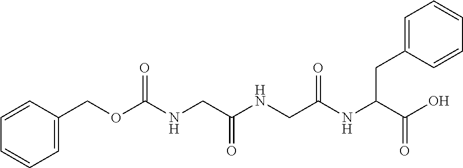 [SMILES: O=C(CNC(=O)OCc1ccccc1)NCC(=O)NC(Cc1ccccc1)C(=O)O] | 413.4292 | | |
| 24 | 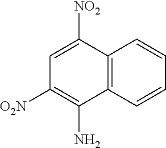 [SMILES: Nc1c([N+](=O)[O-])cc([N+](=O)[O-])c2ccccc12] | 405.3814 | | |
| 25* | 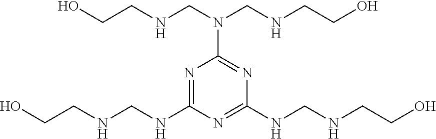 [SMILES: OCCNCNc1nc(NCNCCO)nc(N(CNCCO)CNCCO)n1] | 418.4982 | | |
| 26 | 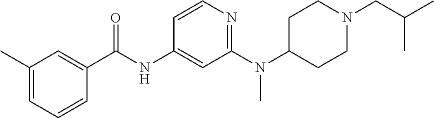 [SMILES: Cc1cccc(C(=O)Nc2ccnc(N(C)C3CCN(CC(C)C)CC3)c2)c1] | 380.5360 | | |
| 27 | 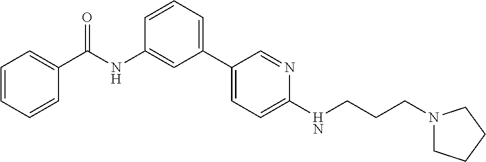 [SMILES: O=C(Nc1cccc(-c2ccc(NCCCN3CCCC3)nc2)c1)c1ccccc1] | 400.5360 | | |
| 28 | 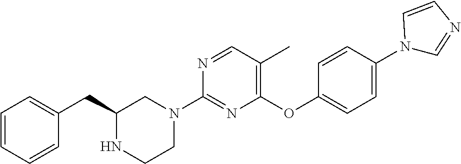 [SMILES: Cc1cnc(N2CCN[C@@H](Cc3ccccc3)C2)nc1Oc1ccc(-n2ccnc2)cc1] | 426.5240 | | |
| 29 | 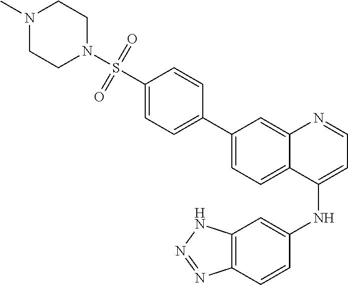 [SMILES: CN1CCN(S(=O)(=O)c2ccc(-c3ccc4c(Nc5ccc6nn[nH]c6c5)ccnc4c3)cc2)CC1] | 499.5930 | + | 4 |
| 30 | 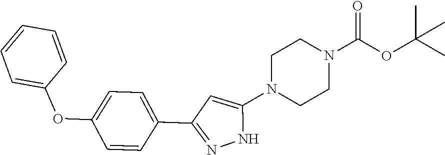 [SMILES: CC(C)(C)OC(=O)N1CCN(c2cc(-c3ccc(Oc4ccccc4)cc3)n[nH]2)CC1] | 420.5130 | + | NA |
| 31 | 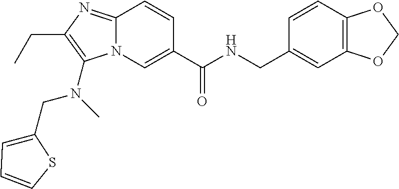 [SMILES: CCc1nc2ccc(C(=O)NCc3ccc4c(c3)OCO4)cn2c1N(C)Cc1cccs1] | 448.5410 | | |
| 32 | 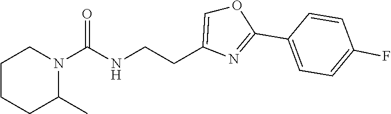 [SMILES: CC1CCCCN1C(=O)NCCc1coc(-c2ccc(F)cc2)n1] | 331.3914 | + | NA |
| 33 | 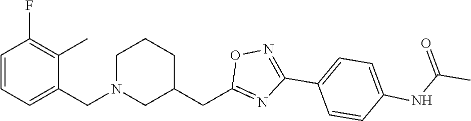 [SMILES: CC(=O)Nc1ccc(-c2noc(CC3CCCN(Cc4cccc(F)c4C)C3)n2)cc1] | 422.5044 | | |
| 34 | 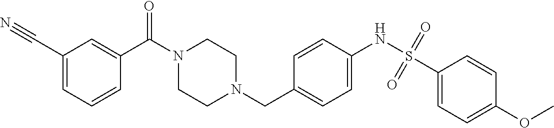 [SMILES: COc1ccc(S(=O)(=O)Nc2ccc(CN3CCN(C(=O)c4cccc(C#N)c4)CC3)cc2)cc1] | 490.57 | | |
| 35 | 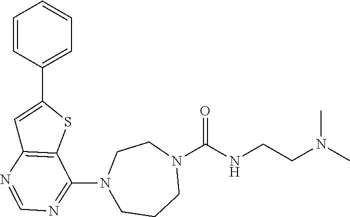 [SMILES: CN(C)CCNC(=O)N1CCCN(c2ncnc3cc(-c4ccccc4)sc23)CC1] | 424.57 | + | NA |
| 36 | 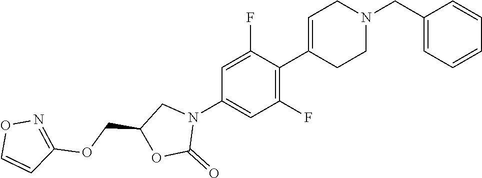 [SMILES: O=C1O[C@@H](COc2ccon2)CN1c1cc(F)c(C2=CCN(Cc3ccccc3)CC2)c(F)c1] | 467.4728 | | |
| 37 | 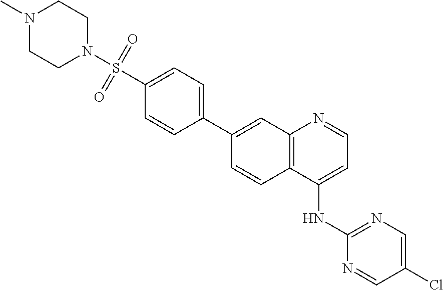 [SMILES: CN1CCN(S(=O)(=O)c2ccc(-c3ccc4c(Nc5ncc(Cl)cn5)ccnc4c3)cc2)CC1] | 494.9980 | | |
| 38 | 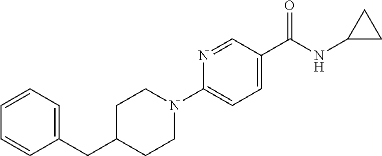 [SMILES: O=C(NC1CC1)c1ccc(N2CCC(Cc3ccccc3)CC2)nc1] | 335.2510 | + | NA |
| 39 | 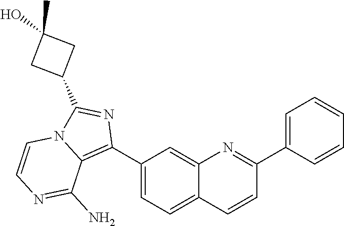 [SMILES: C[C@]1(O)C[C@@H](c2nc(-c3ccc4ccc(-c5ccccc5)nc4c3)c3c(N)nccn32)C1] | 421.5040 | | |
| 40 | 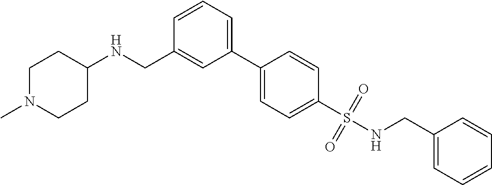 [SMILES: CN1CCC(NCc2cccc(-c3ccc(S(=O)(=O)NCc4ccccc4)cc3)c2)CC1] | 449.6130 | | |
| 41 | 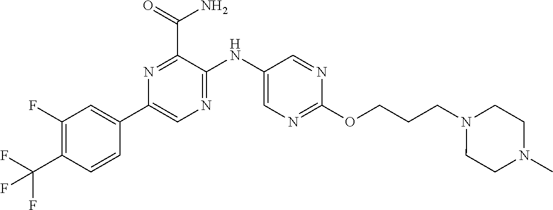 [SMILES: CN1CCN(CCCOc2ncc(Nc3ncc(-c4ccc(C(F)(F)F)c(F)c4)nc3C(N)=O)cn2)CC1] | 534.5196 | | |
| 42 | 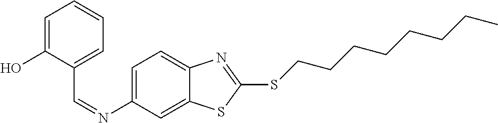 [SMILES: CCCCCCCCSc1nc2ccc(/N=C\c3ccccc3O)cc2s1] | 398.5802 | + | 0.057 |
| 43 | 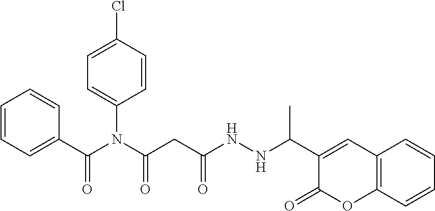 [SMILES: CC(NNC(=O)CC(=O)N(C(=O)c1ccccc1)c1ccc(Cl)cc1)c1cc2ccccc2oc1=O] | 503.9409 | | |
| 44 | 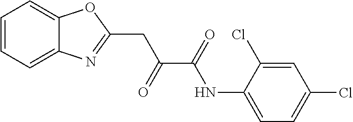 [SMILES: O=C(Cc1nc2ccccc2o1)C(=O)Nc1ccc(Cl)cc1Cl] | 349.1726 | | |
| 45 | 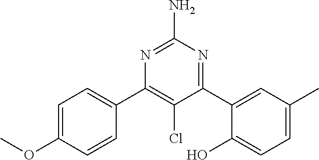 | 341.7963 | + | 0.596 |
| 46 | 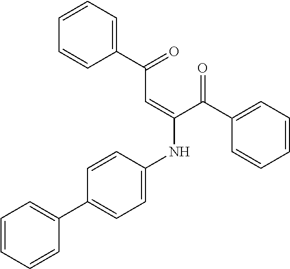 [SMILES: O=C(/C=C(/Nc1ccc(-c2ccccc2)cc1)C(=O)c1ccccc1)c1ccccc1] | 403.4794 | | |
| 47 | 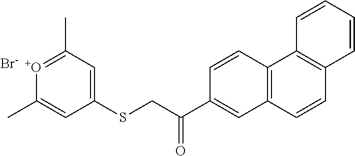 [SMILES: Cc1cc(SCC(=O)c2ccc3c(ccc4ccccc43)c2)cc(C)[o+]1 ; [Br-]] | 438.5519 | + | 0.149 |
| 48 | 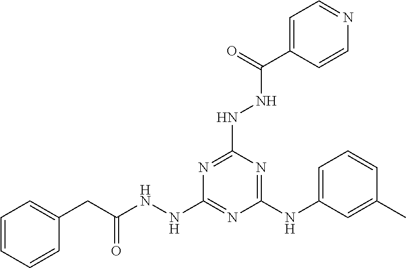 [SMILES: Cc1cccc(Nc2nc(NNC(=O)Cc3ccccc3)nc(NNC(=O)c3ccncc3)n2)c1] | 469.5048 | | |
| 49 | 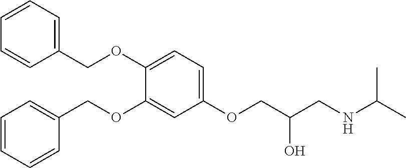 [SMILES: CC(C)NCC(O)COc1ccc(OCc2ccccc2)c(OCc2ccccc2)c1] | 421.5352 | | |
| 50 | 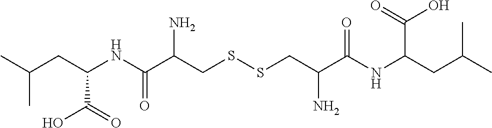 [SMILES: CC(C)CC(NC(=O)C(N)CSSCC(N)C(=O)N[C@@H](CC(C)C)C(=O)O)C(=O)O] | 466.6098 | | |
| 51 | 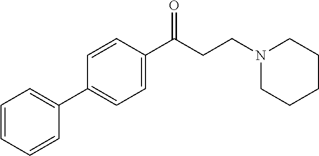 [SMILES: O=C(CCN1CCCCC1)c1ccc(-c2ccccc2)cc1] | 293.4078 | | |
| 52 | 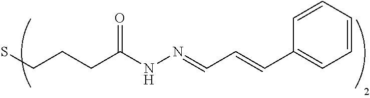 | 462.6086 | | |
| 53 | 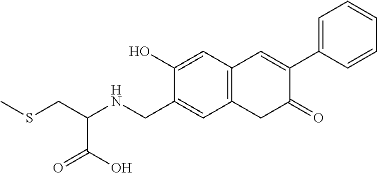 [SMILES: CSCC(NCc1cc2c(cc1O)C=C(c1ccccc1)C(=O)C2)C(=O)O] | 399.4606 | | |
| 54 | 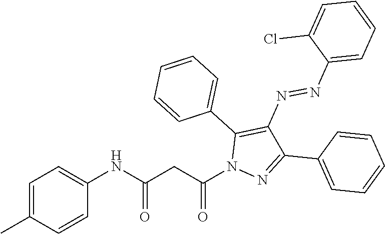 [SMILES: Cc1ccc(NC(=O)CC(=O)n2nc(-c3ccccc3)c(/N=N/c3ccccc3Cl)c2-c2ccccc2)cc1] | 534.0159 | | |
| 55 | 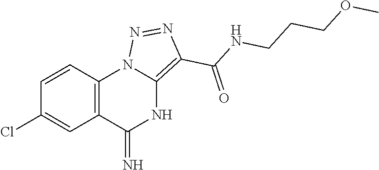 [SMILES: COCCCNC(=O)c1nnn2c1[nH]c(=N)c1cc(Cl)ccc12] | 334.7645 | | |
| 56 | 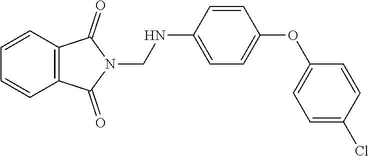 [SMILES: O=C1c2ccccc2C(=O)N1CNc1ccc(Oc2ccc(Cl)cc2)cc1] | 378.8141 | | |
| 57 | 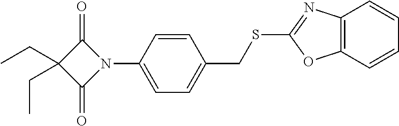 [SMILES: CCC1(CC)C(=O)N(c2ccc(CSc3nc4ccccc4o3)cc2)C1=O] | 380.4620 | | |
| 58 | 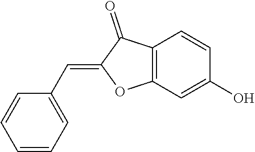 [SMILES: O=C1/C(=C/c2ccccc2)Oc2cc(O)ccc21] | 238.2420 | | |
| 59 | 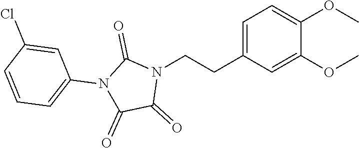 [SMILES: COc1ccc(CCN2C(=O)C(=O)N(c3cccc(Cl)c3)C2=O)cc1OC] | 388.8067 | | |
| 60 | 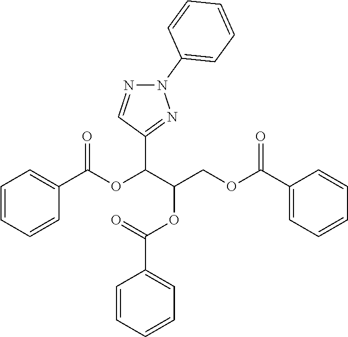 [SMILES: O=C(OCC(OC(=O)c1ccccc1)C(OC(=O)c1ccccc1)c1cnn(-c2ccccc2)n1)c1ccccc1] | 547.566 | | |
| 61 | 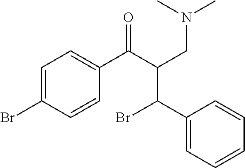 [SMILES: CN(C)CC(C(=O)c1ccc(Br)cc1)C(Br)c1ccccc1] | 425.1622 | | |
| 62 | 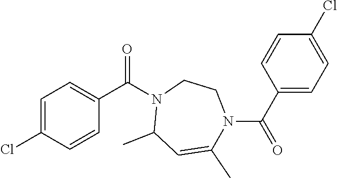 [SMILES: CC1=CC(C)N(C(=O)c2ccc(Cl)cc2)CCN1C(=O)c1ccc(Cl)cc1] | 403.3072 | | |
| 63 | 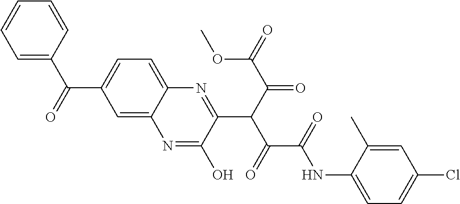 [SMILES: COC(=O)C(=O)C(C(=O)C(=O)Nc1ccc(Cl)cc1C)c1nc2ccc(C(=O)c3ccccc3)cc2nc1O] | 545.9349 | | |
| 64 | 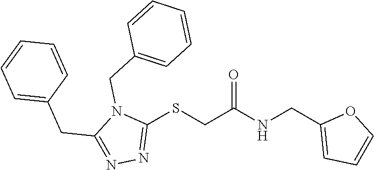 [SMILES: O=C(CSc1nnc(Cc2ccccc2)n1Cc1ccccc1)NCc1ccco1] | 418.5150 | | |
| 65 | 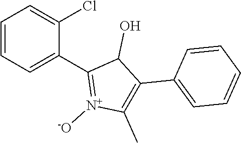 | 299.7540 | | |
| 66 | 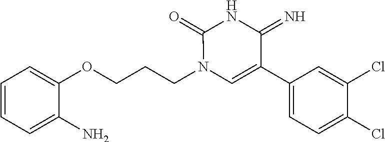 [SMILES: N=c1[nH]c(=O)n(CCCOc2ccccc2N)cc1-c1ccc(Cl)c(Cl)c1] | 405.2828 | | |
| 67 | 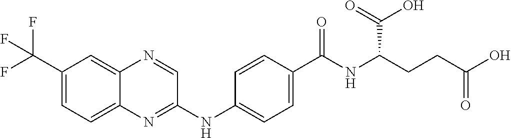 [SMILES: O=C(O)CC[C@H](NC(=O)c1ccc(Nc2cnc3cc(C(F)(F)F)ccc3n2)cc1)C(=O)O] | 462.3843 | | |
| 68 | 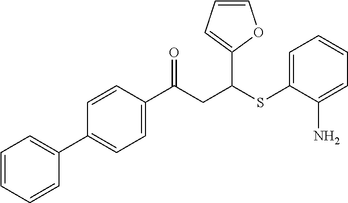 [SMILES: Nc1ccccc1SC(CC(=O)c1ccc(-c2ccccc2)cc1)c1ccco1] | 399.5064 | | |
| 69 | 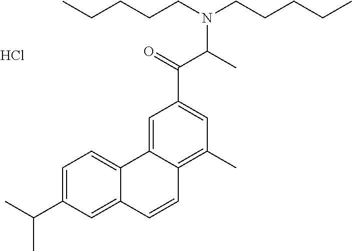 [SMILES: CCCCCN(CCCCC)C(C)C(=O)c1cc(C)c2ccc3cc(C(C)C)ccc3c2c1 ; Cl] | 482.1368 | + | 0.196 |
| 70 | 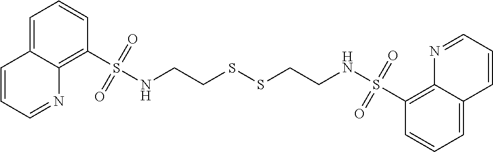 [SMILES: O=S(=O)(NCCSSCCNS(=O)(=O)c1cccc2cccnc12)c1cccc2cccnc12] | 534.6802 | | |
| 71 | 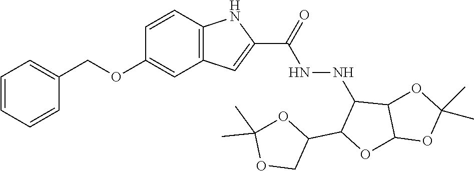 [SMILES: CC1(C)OCC(C2OC3OC(C)(C)OC3C2NNC(=O)c2cc3cc(OCc4ccccc4)ccc3[nH]2)O1] | 523.5860 | + | 0.38 |
| 72 | 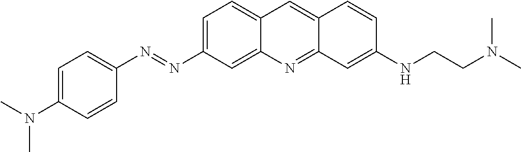 | 412.5364 | | |
| 73 | 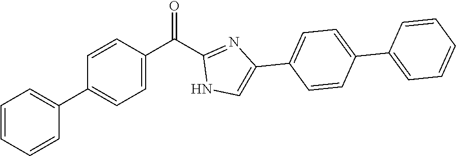 [SMILES: O=C(c1ccc(-c2ccccc2)cc1)c1nc(-c2ccc(-c3ccccc3)cc2)c[nH]1] | 400.4788 | + | 0.149 |
| 74 | 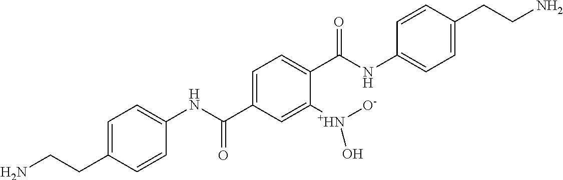 [SMILES: NCCc1ccc(NC(=O)c2ccc(C(=O)Nc3ccc(CCN)cc3)c([NH+]([O-])O)c2)cc1] | 449.5110 | | |
| 75 | 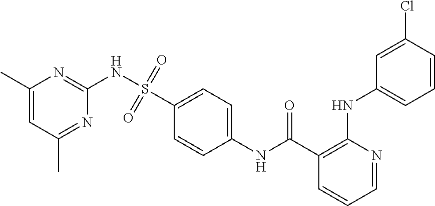 [SMILES: Cc1cc(C)nc(NS(=O)(=O)c2ccc(NC(=O)c3cccnc3Nc3cccc(Cl)c3)cc2)n1] | 508.9813 | + | 1.55 |
| 76 | 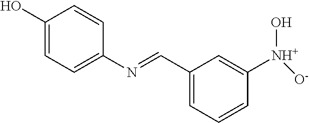 [SMILES: [O-][NH+](O)c1cccc(/C=N/c2ccc(O)cc2)c1] | 244.2500 | | |
| 77 | 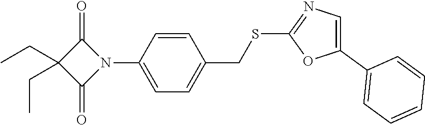 [SMILES: CCC1(CC)C(=O)N(c2ccc(CSc3ncc(-c4ccccc4)o3)cc2)C1=O] | 407.53 | | |
| 78 | 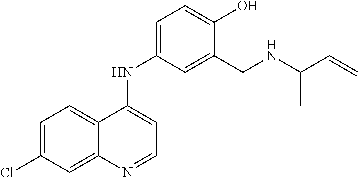 [SMILES: C=CC(C)NCc1cc(Nc2ccnc3cc(Cl)ccc23)ccc1O] | 353.8505 | | |
| 79 | 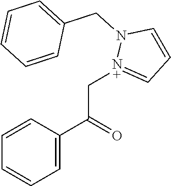 [SMILES: O=C(C[n+]1cccn1Cc1ccccc1)c1ccccc1] | 277.3451 | | |
| 80 | 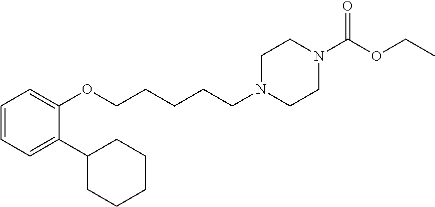 [SMILES: CCOC(=O)N1CCN(CCCCCOc2ccccc2C2CCCCC2)CC1] | 402.5758 | | |
| 81 | 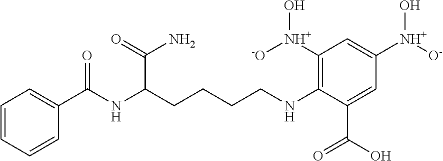 | 463.4460 | | |
| 82 | 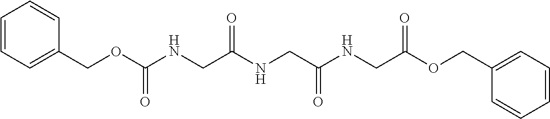 [SMILES: O=C(CNC(=O)CNC(=O)OCc1ccccc1)NCC(=O)OCc1ccccc1] | 413.4292 | | |
| 83 | 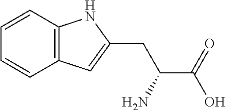 [SMILES: N[C@H](Cc1cc2ccccc2[nH]1)C(=O)O] | 204.2290 | | |
| 84 | 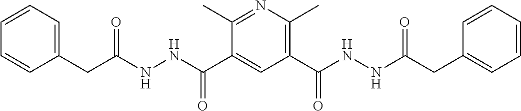 [SMILES: Cc1nc(C)c(C(=O)NNC(=O)Cc2ccccc2)cc1C(=O)NNC(=O)Cc1ccccc1] | 459.5036 | + | 1.09 |
| 85 | 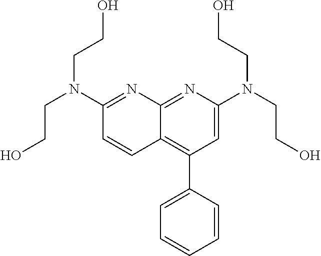 [SMILES: OCCN(CCO)c1ccc2c(-c3ccccc3)cc(N(CCO)CCO)nc2n1] | 412.4876 | | |
| 86 | 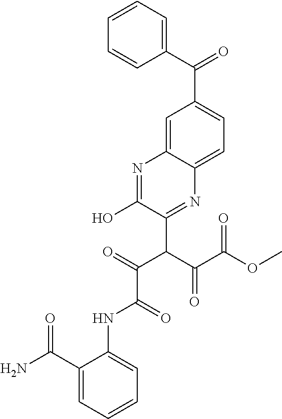 [SMILES: COC(=O)C(=O)C(C(=O)C(=O)Nc1ccccc1C(N)=O)c1nc2ccc(C(=O)c3ccccc3)cc2nc1O] | 540.4880 | | |
| 87 | 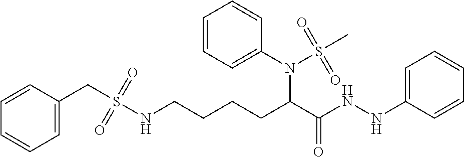 [SMILES: CS(=O)(=O)N(c1ccccc1)C(CCCCNS(=O)(=O)Cc1ccccc1)C(=O)NNc1ccccc1] | 544.6826 | | |
| 88 | 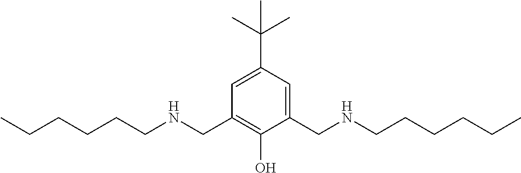 [SMILES: CCCCCCNCc1cc(C(C)(C)C)cc(CNCCCCCC)c1O] | 376.6244 | | |
| 89 | 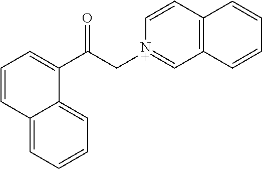 [SMILES: O=C(C[n+]1ccc2ccccc2c1)c1cccc2ccccc12] | 298.3635 | | |
| 90 | 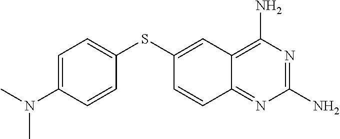 [SMILES: CN(C)c1ccc(Sc2ccc3nc(N)nc(N)c3c2)cc1] | 311.4038 | | |
| 91 | 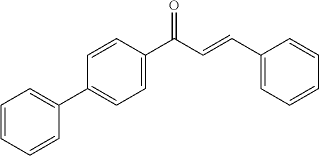 [SMILES: O=C(/C=C/c1ccccc1)c1ccc(-c2ccccc2)cc1] | 284.3568 | | |
| 92 | 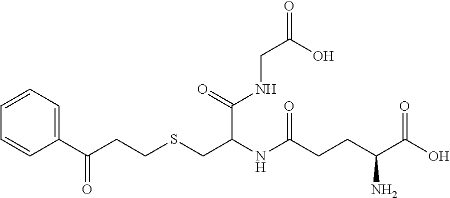 [SMILES: N[C@@H](CCC(=O)NC(CSCCC(=O)c1ccccc1)C(=O)NCC(=O)O)C(=O)O] | 439.4824 | | |
| 93 | 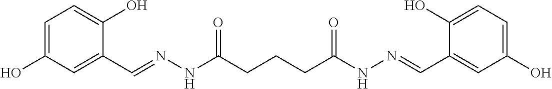 [SMILES: O=C(CCCC(=O)N/N=C/c1cc(O)ccc1O)N/N=C/c1cc(O)ccc1O] | 400.3902 | | |
| 94 | 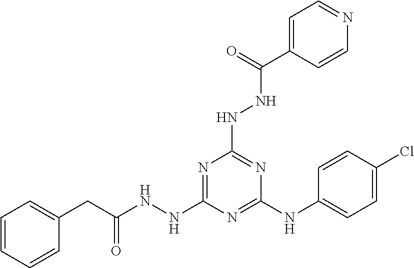 [SMILES: O=C(Cc1ccccc1)NNc1nc(NNC(=O)c2ccncc2)nc(Nc2ccc(Cl)cc2)n1] | 489.9231 | | |

+: positive compounds using 100 μM on HTRF
NA: Not applicable (No dose-response effect)
*Compound 25 is also referred to as compound 95

The obtained data presented in Table A showed that out of the 94 compounds tested, 16 (17%) were able to lead to a 50% reduction of the HTRF signal, as shown in FIGS. 3A-B and summarized in Table B hereinbelow, thus indicating a significant effect on the PD-1/PD-L1 inhibition. FIG. 3A presents the data obtained for the 16 confirmed hits (blue—Compounds 1 and 73, black —Compounds 5, 29, 18, 75, and 84, green —Compounds 45, 42 and 71, yellow —Compounds 69 and 47, and white —Compounds 30, 32, 35 and 38; dark gray —BMS202 positive control). Amongst the 16 compounds, 4 (white —Compounds 30, 32, 35 and 38) were considered false positives. Results were normalized (0-100%) considering PD-1/PD-L1 interaction (light gray) the 100%. Data are presented as mean±SD, N=3, n=3 from three independent experiments performed in triplicate.

TABLE B

| | | Molecular | |
|---|---|---|---|
| | | Weight | IC₅₀ |
| ID | STRUCTURE | [g/mol] | (μM) |
| 1 | 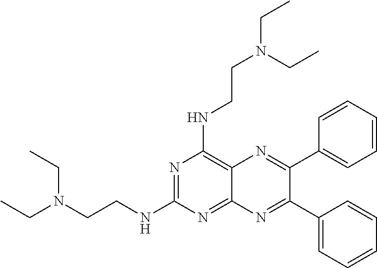 [SMILES: CCN(CC)CCNc1nc(NCCN(CC)CC)c2nc(-c3ccccc3)c(-c3ccccc3)nc2n1] | 512.6996 | 0.186 |
| 5 | 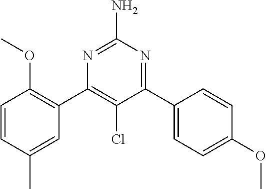 [SMILES: COc1ccc(-c2nc(N)nc(-c3cc(C)ccc3OC)c2Cl)cc1] | 378.4876 | 2.44 |
| 18 | 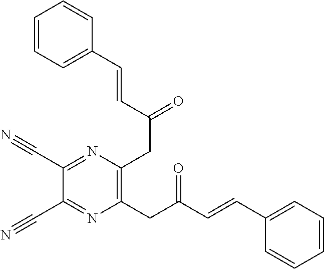 [SMILES: N#Cc1nc(CC(=O)/C=C/c2ccccc2)c(CC(=O)/C=C/c2ccccc2)nc1C#N] | 418.4538 | 0.19 |
| 29 | 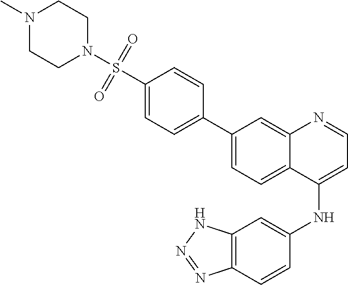 [SMILES: CN1CCN(S(=O)(=O)c2ccc(-c3ccc4c(Nc5ccc6nn[nH]c6c5)ccnc4c3)cc2)CC1] | 499.5930 | 4 |
| 30 | 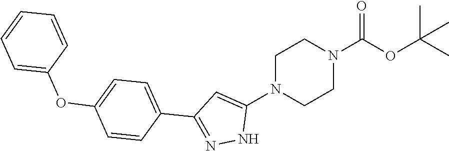 [SMILES: CC(C)(C)OC(=O)N1CCN(c2cc(-c3ccc(Oc4ccccc4)cc3)n[nH]2)CC1] | 420.5130 | NA |
| 32 | 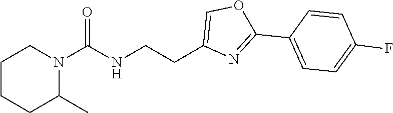 [SMILES: CC1CCCCN1C(=O)NCCc1coc(-c2ccc(F)cc2)n1] | 331.3914 | NA |
| 35 | 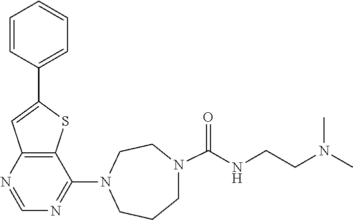 [SMILES: CN(C)CCNC(=O)N1CCCN(c2ncnc3cc(-c4ccccc4)sc23)CC1] | | NA |
| 38 | 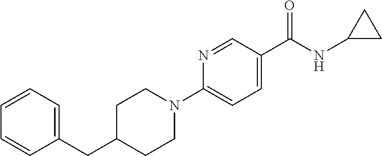 [SMILES: O=C(NC1CC1)c1ccc(N2CCC(Cc3ccccc3)CC2)nc1] | 335.2510 | NA |
| 42 | 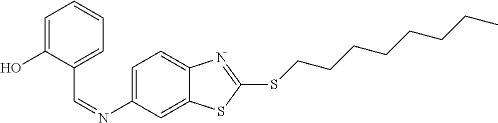 [SMILES: CCCCCCCCSc1nc2ccc(/N=C\c3ccccc3O)cc2s1] | 398.5802 | 0.057 |
| 45 | 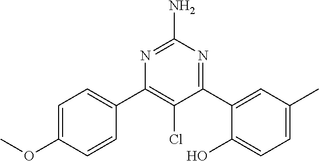 | 341.7963 | 0.596 |
| 47 | 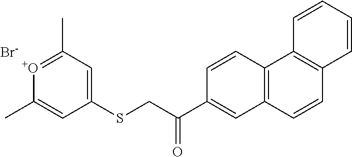 [SMILES: Cc1cc(SCC(=O)c2ccc3c(ccc4ccccc43)c2)cc(C)[o+]1 ; [Br-]] | 438.5219 | 0.149 |
| 69 | 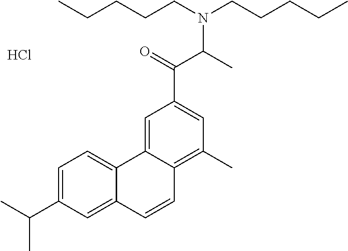 [SMILES: CCCCCN(CCCCC)C(C)C(=O)c1cc(C)c2ccc3cc(C(C)C)ccc3c2c1 ; Cl] | 482.1368 | 0.196 |
| 71 | 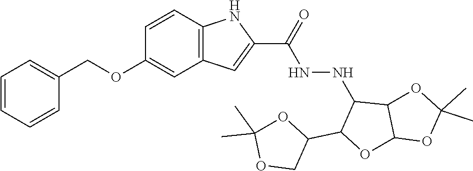 [SMILES: CC1(C)OCC(C2OC3OC(C)(C)OC3C2NNC(=O)c2cc3cc(OCc4ccccc4)ccc3[nH]2)O1] | 521.5688 | 0.38 |
| 73 | 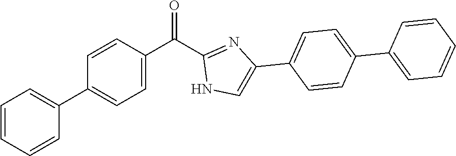 [SMILES: O=C(c1ccc(-c2ccccc2)cc1)c1nc(-c2ccc(-c3ccccc3)cc2)c[nH]1] | 400.4788 | 0.149 |
| 75 | 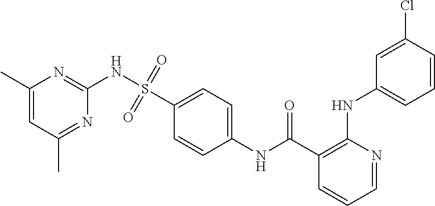 [SMILES: Cc1cc(C)nc(NS(=O)(=O)c2ccc(NC(=O)c3cccnc3Nc3cccc(Cl)c3)cc2)n1] | 508.9813 | 1.55 |
| 84 | 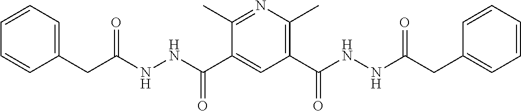 [SMILES: Cc1nc(C)c(C(=O)NNC(=O)Cc2ccccc2)cc1C(=O)NNC(=O)Cc1ccccc1] | 459.5036 | 1.09 |

FIG. 3B is a schematic presentation of the chemical skeleton in each of the groups in FIG. 3A. The chemical structures of the identified validated (hit) compounds is shown in Table B.

Validated hits were tested in three independent assays and further analyzed for dose-response (8 doses in 1:2 and 1:10 serial dilutions) starting from 100 μM, using BMS202 (dark gray) as a positive control for PD-1/PD-L1 inhibition. The obtained data is shown in FIGS. 4A-F. Among the 16 compounds, 12 revealed dose-response effect and were further analyzed for their binding to PD-L1. Since no dose-response was observed for four compounds (30, 32, 35 and 38), it was assumed that they were false-positives and were therefore abandoned. Two compounds (18 and 29) were not further used due to stability issues.

The following IC₅₀ values were determined for each compound and are also presented in Table B: 1 (IC₅₀ 186 nM), 5 (IC₅₀ 2.44 μM), 18 (IC₅₀ 190 nM), 29 (IC₅₀ 4 μM), 42 (IC₅₀ 57 nM), 45 (IC₅₀ 596 nM), 47 (IC₅₀ 149 nM), 69 (IC₅₀ 196 nM), 71 (IC₅₀ 380 nM), 73 (IC₅₀ 149 nM), 75 (IC₅₀ 1.55 μM), 84 (IC₅₀ 1.09 μM), and BMS202 (IC₅₀ 57 nM). Data are presented as mean±SD, N=3, n=9 from three independent experiments performed in triplicate.

Concluding Remarks:

The in silico approach underlying a rational design of PD-1/PD-L1 small-molecule inhibitors was based on the previously reported structural information. In silico studies (structure-based virtual screening using molecular docking) led to the selection of 94 virtual hits presenting good spatial fitting within the PD-L1 pocket, high score values, key interaction to pocket residues, as well as, good ADMET properties. The hit validation achieved 16 (17%) compounds using a standard biochemical fluorescence-based PD-1/PD-L1 binding assay.

### Example 2

### Effect of Hit Compounds on Thermal Stability

Based on the mode of action described for BMS202, it was hypothesized that the identified compounds would stabilize the protein upon binding to PD-L1.

To test this hypothesis, the melting temperature (Tm) of the PD-L1 was determined in thermal denaturation assays by differential scanning fluorimetry (DSF) and checked for a shift in the proteins' Tm (ΔTm) in the presence of the PD-L1 inhibitor candidates. The extent of temperature shift is proportional to the compounds' stabilizing effect and thereby used to infer inhibitors' affinity.

First, the thermal denaturation profile of the recombinant human PD-L1 in buffer conditions was assessed, as well as possible interferences of tested compounds with the fluorescence-dye.

The obtained data are shown in FIG. 5A. Thermal shifts indicate the stabilization of PD-L1 by compounds 5 (black, rectangle), 42 (green), 47 (yellow, black circle), 69 (yellow), 75 (black, triagonal) and 84 (black, circle). Curves represent the fraction of unfolded recombinant human PD-L1 protein, where 0 represents the folded PD-L1 and 1 the unfolded, in the presence of 1% DMSO (light gray), indicated compounds (green, yellow and white) and BMS202 (dark gray) at 100 μM.

The compounds 1, 71 and 73 revealed to interfere and therefore were not included in the DSF studies. The relative fluorescence intensity (RFU) was plotted as a function of temperature and the midpoint of the thermal transition (Tm) was calculated. The observed Tm values were highly reproducible, with a standard deviation of <0.1° C. using different batches of the protein (Tm=54.5° C.). A thermal shift was observed for all tested molecules.

DSF was then used to profile the compounds. The shift of the melting curve in the presence of compounds (100 μM), quantified by the change in melting temperature (ΔTm), suggests increased thermal stability of the protein-compound complex. As shown in FIG. 5B, the tested compounds increased the thermal stability by 3-4° C., confirming the higher stability of the protein in the presence of 100 μM of the tested molecules. The observed thermal shifts range are consistent with the relatively small stabilizing effect that compounds are expected to have on the dimer stabilization formed by the soluble full-length PD-L1 protein.

The compound-binding of small-molecule inhibitors to PD-L1 was further confirmed by a WaterLOGSY NMR assay. As shown in FIG. 5C, in the presence of PD-L1, Compound 69 and BMS202 present similar NMR patterns.

Taken together, these results suggest that the tested compounds have a similar mode of action as BMS202, which bind to PD-L1 and thereby can interfere with its interaction with the PD-1.

### Example 3

### Effect of Hit Compounds on Cell Viability

PD-L1-targeted small molecules are not expected to have a direct effect on targeted cell viability. Therefore, to assess cell tolerance to the compounds, their impact on cell viability using the cell metabolic viability assay (MTT) was tested.

Human breast cancer MDA-MB-231 and melanoma A375 cells were exposed to increasing concentrations (1, 10, and 100 μM) of selected compounds for 48 hours. Cell viability was normalized to untreated cells. Three different concentrations 100 μM (blue), 10 μM (green) and 1 μM (gray) were tested. Data are presented as mean±SD, N=3, n=3 and N=1, n=3, from three or one independent experiment(s) performed in triplicate.

The obtained data is presented in FIG. 6. All cell lines, MDA-MB-231 (ATCC #HTB-26™), A375 (ATCC #CRL. 1619™), and HMEC (ATCC #CRL-3243™) showed tolerance to the compounds.

Some of the tested compounds revealed a considerably different toxicity, compared to the BMS molecule. The BMS202 molecule presented a higher toxicity at the highest concentration, as previously reported [Skalniak, L. et al. 2017, supra].

In general, the tested compounds showed low toxicity, except for compounds 1, 73 and 75 that revealed a toxicity higher or similar to that obtained for BMS202 in both cell lines.

Endothelial cells (HMEC-1) were used to infer a possible off-target systemic toxic effect. The impact of the small molecule hits on cell viability was similar to the ones obtained in both tumor cell lines, except for Compound 69.

Considering the in silico results and the impact of these compounds on cell viability, the compounds 5, 42, 47, 69 and 84 (see, Table B, bolded and underlined compound ID numbers) were considered the most suitable for their further characterization as potential PD-1/PD-L1 inhibitors in vitro and ex vivo.

### Example 4

### In Vitro Modulation of PD-1/PD-L1 Interaction

A cell-based assay was set up to determine the effective compound-activity on PD-1/PD-L1 inhibition in vitro. Based on the absence of reported data on cell-based assays for BMS202 and taking into account the current clinical relevance of immune checkpoint mAb, anti-PD-L1 monoclonal antibody (αPD-L1) was used as reference inhibitor for the subsequent studies.

Cell-surface PD-L1 was determined by flow cytometry.

Initially, four human tumor cell lines were selected for PD-L1 assessment, one breast cancer (MDA-MB-231) cell line and three melanoma (A375, G361, and SK-MEL1) cell lines. The basis for cell line selection was the remarkable results obtained in highly immunogenic tumors, as melanoma, and the exciting outcomes in the treatment of other tumors reported as poorly immunogenic, as breast cancer.

Cells were stimulated with 200 ng/ml IFN-γ (gray) for 18 hours or left untreated (blue). The levels of PD-L1 on these cell lines was assessed by fluorescence-activated cell sorting (FACS).

As shown in FIG. 7A, only the breast cancer cell line presented significant levels of PD-L1. It is noted that it has been reported in the literature that tumor cells within TME enhance antigen presentation upon IFN-γ exposure [Gessani, S., et al. Toxins (Basel), 6, 1615-1643 (2014)]. Accordingly, two non-responsive melanoma cell lines (A375 and SK-MEL1) became PD-L1 positive upon IFN-γ pretreatment. Only one cell line (G361) did not respond to IFN-γ exposure and presented insignificant levels of PD-L1.

For following cellular assays, MDA-MB-231 and A375 were selected as these expressed the highest levels of PD-L1 where PD-L1 inhibition could be effectively observed.

The selected cell lines were incubated with the selected compounds to assess compound-binding affinities, and consequently PD-1/PD-L1 inhibition. The levels of remaining accessible PD-L1 in these cells were evaluated by FACS analysis using a fluorescence PD-1 proxy. To understand compound activity, different incubation times (24, 48 and 72 hours) were evaluated on MDA-MB-231 breast cancer cell line. Cells were pretreated with 10 μM of the selected compounds at different time points, and the obtained data is presented in FIG. 7B. As shown therein, the highest reduction on PD-L1 accessibility upon treatment with the different compounds was observed for the most prolonged incubation time (72 hours), and therefore the subsequent experiments were performed by incubating the compounds for 72 hours.

In order to quantify the impact of the selected compounds on PD-1/PD-L1 inhibition, MDA-MB-231 breast cancer (FIG. 7C) and melanoma A375 (FIG. 7D) cells were incubated with 10 μM of each tested compound (green, yellow, and black), αPD-L1 (red) and BMS202 (dark gray). The remaining accessible PD-L1 was determined by flow cytometry.

As shown in FIGS. 7C and 7D, all compounds demonstrated an impact on PD-L1 accessibility and thus inhibit the PD-1/PD-L1 interaction. Compound 69 was found to be the most active compound (IC₅₀ 1 μM) in both cell lines. The obtained data also showed that BMS202 did not exhibit a significant impact on PD-1/PD-L1 interaction using this cell-based experiment.

The in vitro studies showed that the exemplified PD-L1 binding small molecules were able to considerably impact PD-L1 levels in both breast cancer (MDA-MB-231) and melanoma (A375) cell lines evaluated. In contrast, a less meaningful effect was observed using the BMS202 small-molecule inhibitor.

### Example 5

### Ex Vivo Modulation of PD-1/PD-L1 Interaction

The impact of the hit compounds on T-cell function and ability to infiltrate into patient-derived tumor spheroids was further assessed.

To test the impact of the selected compounds on T-cell activation and function, a co-culture experiment with patient-derived PBMC and autologous tumor cells was established, in accordance with Dijkstra, K. K. et al. Cell 174, 1586-1598.e12 (2018), and as schematically presented in FIG. 8A. Tumor cells were obtained from surgical resections of melanoma (Mel), bone metastases of breast (BBM) and lung cancer (LBM), as shown in Table 1 below.

TABLE 1

| | | | Tumor | Primary tumor/ | Primary |
|---|---|---|---|---|---|
| Sample | Sex | Age | location | metastasis | tumor |
| Mel1a | M | 80 | Anal canal | Primary tumor | Melanoma |
| Mel1b | | | Inguinal region | Metastasis | Melanoma |
| Mel2 | F | 75 | Inguinal region | Primary tumor | Melanoma |
| Mel3 | M | 79 | Right foot | Primary tumor | Melanoma |
| | | | plantar | | |
| Mel4 | M | 89 | Left leg | Metastasis | Melanoma |
| BBM1 | F | 38 | Bone | Metastasis | Breast |
| BBM2 | F | 58 | Bone | Metastasis | Breast |
| LBM1 | M | 60 | Bone | Metastasis | Lung |

Due to limitations on the number of fresh-paired blood and tumor cells obtained from all samples, the PD-1/PD-L1 small-molecule inhibitor Compound 69 was selected to perform these studies.

Prior to co-culture, freshly isolated cells (tumor and PBMC) were characterized by FACS analysis (data not shown), and tumor cells were pre-stimulated with IFN-γ for 18 hours to enhance PD-L1 expression. PBMC were plated with anti-CD28 and interleukin-2 (IL-2) to provide co-stimulation and to support T-cell proliferation, respectively. Effector:Target cells at 2:1 ratio were simultaneously seeded, where Effector cells are T cells and Target cells correspond to tumor cells.

The PBMC and tumor cells were co-cultured and treated with anti-CD28 (blue) and PD-L1 inhibitors (small-molecule inhibitor Compound 69 (yellow) and anti-PD-L1 (red)) for 72 hours. The PD-1/PD-L1 inhibition and T-cell activation were assessed using flow cytometry, and the obtained data is shown in FIGS. 8B-E. The anti-PD-L1 (αPD-L1) was added as the relevant control for PD-L1 inhibition. After 72 hours of incubation, the anti-tumor T-cell based reactivity was inferred by assessing different functional markers (CD45, CD3, CD4, CD8, PD-1, PD-L1, IFN-7 and CD107a) by FACS analysis, as shown in FIG. 8F.

As shown in FIGS. 8B-E, in all 7 (100%) patients, the small-molecule inhibitor Compound 69 induced both PD-1/PD-L1 inhibition, IFN-γ secretion and CD107a upregulation in CD8⁺ T cells after 72 hours of co-culture. The magnitude of the response varied between patients.

Importantly, the tested small-molecule inhibitor reached the same level of PD-1/PD-L1 inhibition as the αPD-L1 mAb (see, FIG. 8B). In contrast to αPD-L1, the small-molecule inhibitor also demonstrated to impact the PD-1 levels. The impact on PD-1 was observed in all patients, with a significant decrease observed using the cells extracted from melanoma patients (see, FIG. 8C). Surprisingly, the small-molecule inhibitor showed higher secretion of IFN-7, CD107a upregulation, and consequently an enhanced T-cell activation than the αPD-L1 in three patient samples (see, FIGS. 8D-F).

Overall, reproducible responses were observed among the different samples, regardless of the type of cancer.

### Example 6

### Small-Molecule Inhibitor Promote T-Cell Infiltration

To evaluate the anti-tumor effect of PD-1/PD-L1 inhibitors, a 3D tumor spheroid model composed of patient-derived melanoma cells (Mel4) from surgical resection, and autologous PBMC was established.

The ability of PD-1/PD-L1 inhibitors (small-molecule inhibitor Compound 69 and αPD-L1) to promote CD8⁺ T-cell infiltration into the tumor site was evaluated using a 3D co-culture system. Melanoma spheroids, formed by 5000 patient-derived tumor cells, were embedded in Matrigel, co-cultured with autologous PBMC and treated with small-molecule inhibitor Compound 69, αPD-L1, or left untreated by 72 hours. Cells were grown together in reduced growth factor Matrigel. The spheres and PBMC were either not treated or treated with anti-PD-L1 (αPD-L1) or small-molecule inhibitor Compound 69. The CD8⁺ T-cell infiltration (green) was evaluated after 72 hours of co-culture by confocal microscopy. Scale bar=100 μm.

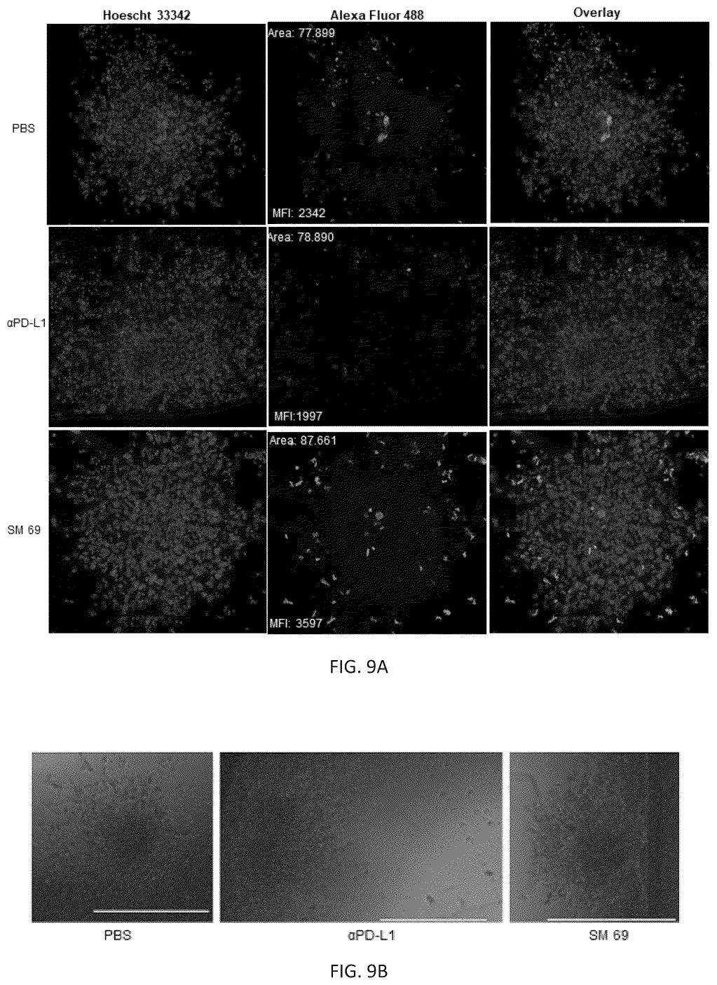

As shown in FIG. 9A, PD-1/PD-L1 inhibition with the small-molecule inhibitor Compound 69 resulted in higher CD8⁺ T cell infiltration within the tumor spheroid compared to the control treatment (αPD-L1) and untreated melanoma cells. As shown in FIG. 9B, the spheroids treated with the small-molecule Compound 69 strongly inhibited the sprouting of patient-derived melanoma cells compared to the control treatment (αPD-L1) and untreated cells.

These data indicate that sprouting of patient-derived melanoma cells can be inhibited by small-molecule via T cells infiltration into patient-derived solid tumor-based spheroid, which making it a prominent candidate for further experiments.

### Example 7

### Small-Molecule Inhibitor Recruit Cytotoxic T-Cells into the TME

To extend the clinical relevance of the ex vivo findings described in Example 6, small-molecule inhibitor Compound 69 was tested using a human-relevant in vivo model.

Accordingly, humanized PD-1 mice were implanted with the colorectal cancer cell line MC38 expressing human PD-L1 (MC38-hPD-L1). The treatment course, as FIG. 10A depicts, started approx. 12 days later, once the animals presented palpable tumors with an average volume of 60 mm³. The animals were treated with the clinically relevant αPD-L1 antibody (atezolizumab) or with small-molecule inhibitor Compound 69 at 10 mg/kg for 10 days. Treatment with Compound 69 has resulted in 93.6% tumor volume reduction relative to vehicle control, as shown in FIG. 10B.

Treating the animals with the small-molecule inhibitor Compound 69 exhibited a similar response to that of the αPD-L1 antibody. As presented in FIGS. 10B and 10C, the tumor growth inhibition was significant for animals treated with both PD-L1 targeting molecules (P<0.001).

At day 30 the animals were euthanized and the TME was characterize by FACS analysis. The data, presented in FIGS. 11A-D, show that the decrease in PD-L1 was associated with the reduced tumor volumes tumor growth, therefore directly correlate tumor grown with PD-L1 levels. These results were accompanied by a higher infiltration of T-cells. In addition, a significant increase in cytotoxic CD8⁺ tumor-infiltrating lymphocytes was observed following treatment with Compound 69 (FIG. 11B; P<0.001).

These data demonstrate that small-molecules inhibit the PD-1/PD-L1 interaction, followed by activation of T-cell function and recruiting of cytotoxic T lymphocyte (CTL) to the TME, which results in a strong control of tumor growth.

### Example 8

### Concluding Insights

The ultimate role of the most promising small-molecule inhibitors in T-cell activation using 2D and 3D co-culture studies of paired matched patient-derived tumor cells and PBMC was assessed. Only tumor cells and PBMC of the same patient were co-cultured to ensure that an HLA-mismatch reaction did not occur, as well as to overcome the subsequent unspecific T-cell activation. In contrast to tumor cell lines, patient-specific model systems are proving to be a most valuable tool in the field of immuno-oncology due to the inherent diversity of the disease and the multifactorial nature of T cell-mediated tumor destruction. The conducted experiments provide a proof of concept that samples treated with the most promising PD-1/PD-L1 inhibitor could activate T cells by inhibiting this pathway. Furthermore, the co-culture of 3D melanoma spheroids and PBMC demonstrated the capacity of small molecules to promote T-cell infiltration. The higher levels of T-cell infiltration may be explained by the possibility offered by small molecules, as opposed to antibodies, to target PD-L1.

Data from in vivo experiments support the inhibition of PD-1/PD-L1 interaction by small-molecule and show it significantly outperforms a monoclonal antibody in recruiting cytotoxic T-cells into the TME. 93.6% tumor volume reduction was obtained using an exemplary small-molecule compound according to some of the present embodiments when compared with vehicle control.

Recently, several studies have demonstrated that there are different cellular sources for PD-L1 (e.g. dendritic cells, or tumor infiltrating lymphocytes) and there is also an intracellular receptor that antibody-based drugs cannot target. Thus, using a small-molecule approach, “any” PD-L1 can be targeted. This is one of the most significant advantages of small molecules over monoclonal antibodies.

Although small molecules needed to be administrated at higher concentrations when compared to monoclonal antibodies, the overall effect on tumor growth and related T-cell activation induced by the exemplary small-molecule inhibitor was equal to or higher than the clinically-relevant αPD-L1.

Overall, these findings show that small molecules can be as effective as monoclonal antibodies, while additionally allowing a considerably higher infiltration of CD8⁺ T cells into patient-derived solid tumor-based spheroid, thereby supporting the promising clinical translation of these small-molecule candidates.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

What is claimed is:

1. A method of treating cancer and/or of interfering with an interaction between PD1 and PD-L1 and/or of increasing T-cell function (e.g. TGF-β), and/or of treating a medical condition associate with PD1, PD-L1 and/or T-cell function on cells, in a subject in need thereof, the method comprising administering to the subject a therapeutically effective amount of a compound represented by Formula I: 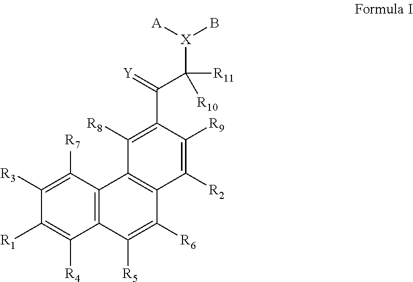
or a pharmaceutically acceptable salt thereof,
wherein:
R₁-R₁₁ are each independently selected from hydrogen, alkyl, cycloalkyl, aryl, heteroaryl, heteroalicyclic, alkoxy, aryloxy, hydroxy, thiol, thioalkoxy, thioaryloxy, amine, imine, halo, nitro, nitrile (cyano), amide, hydrazine, hydrazide, carboxylate, thiocarboxylate, carbamate, thiocarbamate, sulfonyl, sulfinyl, sulfonamide, carbonate, thiocarbonate, sulfoxide, thiosulfate, sulfate, sulfite, thiosulfite, phosphonate, urea, thiourea, guanyl and guanidyl;
Y is O or S;
X is O, S or N, wherein when X is O or S, B is absent; and
A and B, if present, are each independently selected from hydrogen, alkyl, cycloalkyl, aryl, heteroaryl, and heteroalicyclic, said alkyl, cycloalkyl, aryl, heteroaryl, and heteroalicyclic being independently substituted or unsubstituted.

2. The method of claim 1, wherein R₁ and R₂ are each independently selected from hydrogen, alkyl and cycloalkyl, or from hydrogen and alkyl.

3. The method of claim 1, wherein R₁ and/or R₂ is alkyl.

4. The method of claim 1, wherein R₁, R₂ and R₁₁ are each independently an alkyl, and R₁₀ is hydrogen.

5. The method of claim 1, wherein X is N, and A and B are each independently an alkyl.

6. The method of claim 1, wherein X is S and A is a heteroaryl.

7. The method of claim 1, wherein Y is O.

8. The method of claim 1, wherein said cancer is characterized by overexpression of PD-1.

9. The method of claim 1, wherein said medical condition is selected from a neurodegenerative disease or disorder and an infectious disease or disorder.

10. A method of treating cancer and/or of interfering with an interaction between PD1 and PD-L1 and/or of increasing T-cell function (e.g. TGF-β), and/or of treating a medical condition associate with PD1, PD-L1 and/or T-cell function on cells, in a subject in need thereof, the method comprising administering to the subject a therapeutically effective amount of a compound represented by Formula II: 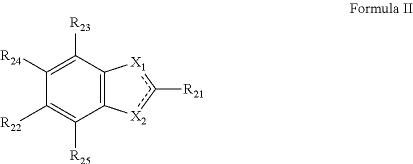
or a pharmaceutically acceptable salt thereof,
wherein:
X₁ and X₂ are each independently selected from N, NR₂₈, S, O, CR₂₆, and CR₂₆R₂₇, at least one of X₁ and X₂ being N, NR₂₈, S or O, and wherein each of the dashed lines represents an optional bond (forming a double bond) when the adjacent X₁ or X₂ is N or CR₂₆;
R₂₈ is hydrogen, alkyl, cycloalkyl or aryl; and
R₂₁-R₂₇ are each independently selected from hydrogen, alkyl, cycloalkyl, aryl, hydroxy, alkoxy, aryloxy, thiol, thioalkoxy, thioaryloxy, amine, imine, halo, nitrile (cyano), nitro, amide, hydrazine, hydrazide, carboxylate, thiocarboxylate, carbamate, thiocarbamate, sulfonyl, sulfinyl, sulfonamide, carbonate, thiocarbonate, sulfoxide, thiosulfate, sulfate, sulfite, thiosulfite, phosphonate, urea, thiourea, guanyl and guanidyl,
provided that at least one and preferably both of R₂₁ and R₂₂ is a heteroatom-containing moiety such as alkoxy, aryloxy, thiol, thioalkoxy, thioaryloxy, amine, imine, nitrile (cyano), amide, hydrazine, hydrazide, carboxylate, thiocarboxylate, carbamate, thiocarbamate, sulfonyl, sulfinyl, and/or sulfonamide.

11. The method of claim 10, wherein:
X₁ is N and X₂ is S; or
X₁ is NR₂₈ and X₂ is CR₂₆.

12. The method of claim 10, wherein said cancer is characterized by overexpression of PD-1.

13. The method of claim 10, wherein said medical condition is selected from a neurodegenerative disease or disorder and an infectious disease or disorder.

14. A method of treating cancer and/or of interfering with an interaction between PD1 and PD-L1 and/or of increasing T-cell function (e.g. TGF-β), and/or of treating a medical condition associate with PD1, PD-L1 and/or T-cell function on cells, in a subject in need thereof, the method comprising administering to the subject a therapeutically effective amount of a compound represented by Formula III: 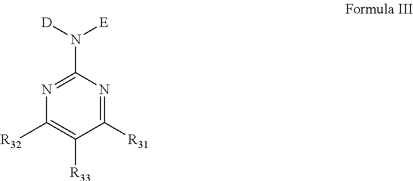
or a pharmaceutically acceptable salt thereof,
wherein:
R₃₃ is hydrogen, alkyl, cycloalkyl, aryl, halo, amine, hydroxy, thiol, aryl, alkoxy, thioalkoxy, aryloxy, thioaryloxy, or, alternatively, forms a cyclic ring with R₃₁ or R₃₂;
R₃₁ and R₃₂ are each independently selected from hydrogen, halo, alkyl, aryl, and amine, or, alternatively, one of R₃₁ and R₃₂ forms a cyclic ring with R₃₃; and
D and E are each independently selected from hydrogen, alkyl, cycloalkyl, aryl, heteroaryl, carbonyl (carbonate), thiocarbonyl (thiocarbonate), carboxylate, thiocarboxylate, sulfonyl, sulfinyl, sulfoxide, thiosulfate, sulfate, sulfite, thiosulfite, urea, thiourea, guanyl and guanidyl, wherein at least one or at least two of R₃₁-R₃₃, D and E is or comprises an aryl, for use in treating cancer and/or for use in interfering with an interaction between PD1 and PD-L1 and/or for use in increasing T-cell function (e.g. TGF-β), and/or for use in treating a medical condition associate with PD1, PD-L1 and/or T-cell function on cells, in a subject in need thereof.

15. The method of claim 14, wherein:
at least one or both of R₃₁ and R₃₃ is an aryl, preferably substituted by hydroxy and/or alkoxy, and at least one or each of D and E is hydrogen; or
at least one or both of R₃₁ and R₃₃ is an alkyl and at least one of D and E is an arylosulfonyl, optionally substituted by an amide; or
R₃₁ and R₃₂ form a nitrogen-containing heteroaryl, R₃₂ is an amine, and at least one of D and E is an alkyl.

16. The method of claim 14, wherein said cancer is characterized by overexpression of PD-1.

17. The method of claim 14, wherein said medical condition is selected from a neurodegenerative disease or disorder and an infectious disease or disorder.

18. A method of treating cancer and/or of interfering with an interaction between PD1 and PD-L1 and/or of increasing T-cell function (e.g. TGF-β), and/or of treating a medical condition associate with PD1, PD-L1 and/or T-cell function on cells, in a subject in need thereof, the method comprising administering to the subject a therapeutically effective amount of a compound represented by Formula IV: 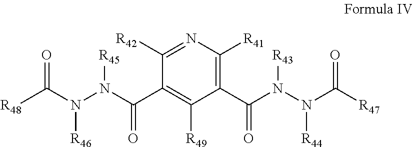
or a pharmaceutically acceptable salt thereof,
wherein:
R₄₃-R₄₆ are each independently hydrogen, alkyl, cycloalkyl and aryl;
R₄₁, R₄₂ and R₄₉ are each independently selected from hydrogen, alkyl, cycloalkyl, aryl, hydroxy, alkoxy, aryloxy, thiol, thioalkoxy, thioaryloxy, amine, imine, halo, nitrile (cyano), nitro, amide, hydrazine, hydrazide, carboxylate, thiocarboxylate, carbamate, thiocarbamate, sulfonyl, sulfinyl, sulfonamide, carbonate, thiocarbonate, sulfoxide, thiosulfate, sulfate, sulfite, thiosulfite, phosphonate, urea, thiourea, guanyl and guanidyl; and
R₄₇ and R₄₈ are each independently selected from alkyl, cycloalkyl, aryl, heteroaryl and heteroalicyclic,
for use in treating cancer and/or for use in interfering with an interaction between PD1 and PD-L1 and/or for use in increasing T-cell function (e.g. TGF-β), and/or for use in treating a medical condition associate with PD1, PD-L1 and/or T-cell function on cells, in a subject in need thereof.

19. The method of claim 18, wherein said cancer is characterized by overexpression of PD-1.

20. The method of claim 18, wherein said medical condition is selected from a neurodegenerative disease or disorder and an infectious disease or disorder.

21. A method of treating cancer and/or of interfering with an interaction between PD1 and PD-L1 and/or of increasing T-cell function (e.g. TGF-β), and/or of treating a medical condition associate with PD1, PD-L1 and/or T-cell function on cells, in a subject in need thereof, the method comprising administering to the subject a therapeutically effective amount of a compound selected from Compounds 1, 5, 18, 29, 42, 45, 47, 69, 71, 73, 75 and 84, as presented in Table B.

22. The method of claim 21, wherein the compound is selected from Compounds 5, 42, 47, 69, 75 and 84, as presented in Table B.

23. The method of claim 21, wherein said cancer is characterized by overexpression of PD-1.

24. The method of claim 21, wherein said medical condition is selected from a neurodegenerative disease or disorder and an infectious disease or disorder.
